(19) United States
(12) Patent Application Publication
(10) Pub. No.: US 2003/0007978 A1
(43) Pub. Date: Jan. 9, 2003

(54) Recombinant MHC molecules useful for manipulation of antigen-specific T-cells

(76) Inventors: Gregory G. Burrows, Portland, OR (US); Arthur A. Vandenbark, Portland, OR (US)

Correspondence Address: KLARQUIST SPARKMAN, LLP, 121 SW SALMON STREET, SUITE 1600, PORTLAND, OR, 97204, US

(21) Appl. No.: 09/847,172

(22) Filed: May 1, 2001

Related U.S. Application Data
(63) Continuation-in-part of application No. 09/153,586, filed on Sep. 15, 1998, now Pat. No. 6,270,772.
(60) Provisional application No. 60/064,552, filed on Sep. 16, 1997.
(60) Provisional application No. 60/064,555, filed on Oct. 10, 1997.
(60) Provisional application No. 60/200,942, filed on May 1, 2000.

(51) Int. Cl. A61K 39/395; C07K 14/74

(52) U.S. Cl. 424/185.1; 530/350

(57) ABSTRACT

Two-domain MHC polypeptides useful for manipulation of antigen-specific T-cells are disclosed. These polypeptides include MHC class II-based molecules that comprise covalently linked β1 and α1 domains, and MHC class I-based molecules that comprise covalently linked α1 and α2 domains. These polypeptides may also include covalently linked antigenic determinants, toxic moieties, and/or detectable labels. The disclosed polypeptides can be used to target antigen-specific T-cells, and are useful, among other things, to detect and purify antigen-specific T-cells, to induce or activate T-cells, and to treat conditions mediated by antigen-specific T-cells.

### PRIORITY CLAIM

This application is a continuation-in-part of co-pending U.S. patent application Ser. No. 09/153,586, filed Sep. 15, 1998, which claims priority to U.S. Provisional Patent Application No. 60/064,552, filed Sep. 16, 1997, and U.S. Provisional Patent Application No. 60/064,555, filed Oct. 10, 1997, all of which are incorporated herein by reference. This application also claims priority to U.S. Provisional Patent Application No. 60/200,942, filed May 1, 2000.

### BACKGROUND

The initiation of an immune response against a specific antigen in mammals is brought about by the presentation of that antigen to T-cells. An antigen is presented to T-cells in the context of a major histocompatibility (MHC) complex. MHC complexes are located on the surface of antigen presenting cells (APCs); the 3-dimensional structure of MHCs includes a groove or cleft into which the presented antigen fits. When an appropriate receptor on a T-cell interacts with the MHC/antigen complex on an APC in the presence of necessary co-stimulatory signals, the T-cell is stimulated, triggering various aspects of the well characterized cascade of immune system activation events, including induction of cytotoxic T-cell function, induction of B-cell function and stimulation of cytokine production.

There are two basic classes of MHC molecules in mammals, MHC class I and MHC class II. Both classes are large protein complexes formed by association of two separate proteins. Each class includes trans-membrane domains that anchor the complex into the cell membrane. MHC class I molecules are formed from two non-covalently associated proteins, the α chain and β2-microglobulin. The a chain comprises three distinct domains, α1, α2 and α3. The three-dimensional structure of the α1 and α2 domains forms the groove into which antigenq fit for presentation to T-cells. The α3 domain is an Ig-fold like domain that contains a trans-membrane sequence that anchors the α chain into the cell membrane of the APC. MHC class I complexes, when associated with antigen (and in the presence of appropriate co-stimulatory signals) stimulate CD8 cytotoxic T-cells, which function to kill any cell which they specifically recognize.

The two proteins which associate non-covalently to form MHC class II molecules are termed the α and β chains. The α chain comprises α1 and α2 domains, and the β chain comprises β1 and β2 domains. The cleft into which the antigen fits is formed by the interaction of the α1 and β1 domains. The α2 and β2 domains are trans-membrane Ig-fold like domains that anchors the α and β chains into the cell membrane of the APC. MHC class II complexes, when associated with antigen (and in the presence of appropriate co-stimulatory signals) stimulate CD4 T-cells. The primary functions of CD4 T-cells are to initiate the inflammatory response, to regulate other cells in the immune system, and to provide help to B cells for antibody synthesis.

The genes encoding the various proteins that constitute the MHC complexes have been extensively studied in humans and other mammals. In humans, MHC molecules (with the exception of class I β2-microglobulin) are encoded in the HLA region, which is located on chromosome 6 and constitutes over 100 genes. There are 3 class I MHC cc chain protein loci, termed HLA-A, -B and -C. There are also 3 pairs of class II MHC a and P chain loci, termed HLA-DR(A and B), HLA-DP(A and B), and HLA-DQ(A and B). In rats, the class I cc gene is termed RT1.A, while the class II genes are termed RT1.B α and RT1.B β. More detailed background information on the structure, function and genetics of MHC complexes can be found in Immunobiology: The Immune System in Health and Disease by Janeway and Travers, Cuurent Biology Ltd./Garland Publishing, Inc. (1997) (ISBN 0-8153-2818-4), and in Bodmer et al. (1994) “Nomenclature for factors of the HLA system” Tissue Antigens vol. 44, pages 1-18 (with periodic updates).

The key role that MHC complexes play in triggering immune recognition has led to the development of methods by which these complexes are used to modulate the immune response. For example, activated T-cells which recognize “self” antigens (autoantigens) are known to play a key role in autoimmune diseases (such as rheumatoid arthritis and multiple sclerosis). Building on the observation that isolated MHC class II molecules (loaded with the appropriate antigen) can substitute for APCs carrying the MHC class II complex and can bind to antigen-specific T-cells, a number of researchers have proposed that isolated MHC/antigen complexes may be used to treat autoimmune disorders. Thus U.S. Pat. Nos. 5,194,425 (Sharma et al.) and 5,284,935 (Clark et al.) disclose the use of isolated MHC class II complexes loaded with a specified autoantigen and conjugated to a toxin to eliminate T-cells that are specifically immunoreactive with autoantigens. In another context, it has been shown that the interaction of isolated MHC II/antigen complexes with T-cells, in the absence of co-stimulatory factors, induces a state of non-responsiveness known as anergy. (Quill et al., J. Immunol., 138:3704-3712 (1987)). Following this observation, Sharma et al. (U.S. Pat. Nos. 5,468,481 and 5,130,297) and Clarke et al. (U.S. Pat. No. 5,260,422) have suggested that such isolated MHC II/antigen complexes may be administered therapeutically to anergize T-cell lines which specifically respond to particular autoantigenic peptides.

Methods for using isolated MHC complexes in the detection, quantification and purification of T-cells which recognize particular antigens have been studied for use in diagnostic and therapeutic applications. By way of example, early detection of T-cells specific for a particular autoantigen would facilitate the early selection of appropriate treatment regimes. The ability to purify antigen-specific T-cells would also be of great value in adoptive immunotherapy. Adoptive immunotherapy involves the removal of T-cells from a cancer patient, expansion of the T-cells in vitro and then reintroduction of the cells to the patient (see U.S. Pat. No. 4,690,915; Rosenberg et al. New Engl. J. Med. 319:1676-1680 (1988)). Isolation and expansion of cancer specific T-cells with inflammatory properties would increase the specificity and effectiveness of such an approach.

To date, however, attempts to detect, quantify or purify antigen specific T-cells using isolated MHC/antigen complexes have not met with widespread success because, among other reasons, binding between the T-cells and such isolated complexes is transient and hence the T-cell/MHC/antigen complex is unstable. In an attempt to address these problems, Altman et al. (Science 274, 94-96 (1996) and U.S. Pat. No. 5,635,363) have proposed the use of large, covalently linked multimeric structures of MHC/antigen complexes to stabilize this interaction by simultaneously binding to multiple T-cell receptors on a target T-cell.

Although the concept of using isolated MHC/antigen complexes in therapeutic and diagnostic applications holds great promise, a major drawback to the various methods reported to date is that the complexes are large and consequently difficult to produce and to work with. While the complexes can be isolated from lymphocytes by detergent extraction, such procedures are inefficient and yield only small amounts of protein. The cloning of the genes encoding the various MHC complex subunits has facilitated the production of large quantities of the individual subunits through expression in prokaryotic cells, but the assembly of the individual subunits into MHC complexes having the appropriate conformational structure has proven difficult.

### SUMMARY

This invention is founded on the discovery that mammalian MHC function can be mimicked through the use of recombinant polypeptides that include only those domains of MHC molecules that define the antigen binding cleft. Specifically, human MHC function can be mimicked through the use of these recombinant polypeptides. These molecules are useful to detect, quantify and purify antigen-specific T-cells. The molecules provided herein may also be used in clinical and laboratory applications to detect, quantify and purify antigen-specific T-cells, induce anergy in T-cells, or to induce T suppressor cells, as well as to stimulate T-cells, and to treat diseases mediated by antigen-specific T-cells.

It is shown herein that antigen-specific T-cell binding can be accomplished with a monomeric molecule comprising, in the case of human class II MHC molecules, only the α1 and β1 domains in covalent linkage (and in some examples in association with an antigenic determinant). For convenience, such MHC class II polypeptides are hereinafter referred to as “β1α1”. Equivalent molecules derived from human MHC class I molecules are also provided herein. Such molecules comprise the α1 and α2 domains of class I molecules in covalent linkage and in association with an antigenic determinant. Such MHC class I polypeptides are referred to as “α1α2”. These two domain molecules may be readily produced by recombinant expression in prokaryotic or eukaryotic cells, and readily purified in large quantities. Moreover, these molecules may easily be loaded with any desired peptide antigen, making production of a repertoire of MHC molecules with different T-cell specificities a simple task.

It is shown that, despite lacking the Ig fold domains and trans-membrane portions that are part of intact MHC molecules, these two domain MHC molecules refold in a manner that is structurally analogous to “whole” MHC molecules, and bind peptide antigens to form stable MHC/antigen complexes. Moreover, these two domain MHC/epitope complexes bind T-cells in an epitope-specific manner, and inhibit epitope-specific T-cell proliferation in vitro. In addition, administration of human β1α1 molecules loaded with an antigenic epitope, such as an epitope of myelin basic protein (MBP), induces a variety of T cell transduction processes and modulates effector functions, including the cytokine and proliferation response. Thus, the two domain MHC molecules display powerful and epitope-specific effects on T-cell activation resulting in secretion of anti-inflammatory cytokines. As a result, the disclosed MHC molecules are useful in a wide range of both in vivo and in vitro applications.

Various formulations of these human two domain molecules are provided by the invention. In their most basic form, human two domain MHC class II molecules comprise β1 and α1 domains of a mammalian MHC class II molecule wherein the amino terminus of the α1 domain is covalently linked to the carboxy terminus of the β1 domain and wherein the polypeptide does not include the α2 or β2 domains. The human two domain MHC class I molecules comprise α1 and α2 domains of a mammalian class I molecule, wherein the amino terminus of the α2 domain is covalently linked to the carboxy terminus of the α1 domain, and wherein the polypeptide does not include an MHC class I α3 domain. For most applications, these molecules are associated, by covalent or non-covalent interaction, with an antigenic determinant, such as a peptide antigen. In certain embodiments, the peptide antigen is covalently linked to the amino terminus of the β1 domain of the class II molecules, or the α1 domain of the class I molecules. The two domain molecules may also comprise a detectable marker, such as a fluorescent label or a toxic moiety, such as ricin A, or an antigen, such as myelin basic protein.

Also provided are nucleic acid molecules that encode the human two domain MHC molecules, as well as expression vectors that may be conveniently used to express these molecules. In particular embodiments, the nucleic acid molecules include sequences that encode the antigenic peptide as well as the human two domain MHC molecule. For example, one such nucleic acid molecule may be represented by the formula Pr-P-B-A, wherein Pr is a promoter sequence operably linked to P (a sequence encoding the peptide antigen), B is the class I α1 or the class II β1 domain, and A is the class I α2 domain or the class II α1 domain. In these nucleic acid molecules, P, B and A comprise a single open reading frame, such that the peptide and the two human MHC domains are expressed as a single polypeptide chain. In one embodiment, B and A are connected by a linker.

In vitro, the human two domain MHC molecules may be used to detect and quantify T-cells, and regulate T-cell function. Thus, such molecules loaded with a selected antigen may be used to detect, monitor and quantify a population of T-cells that are specific for that antigen. The ability to do this is beneficial in a number of clinical settings, such as monitoring the number of tumor antigen-specific T-cells in blood removed from a cancer patient, or the number of self-antigen specific T-cells in blood removed from a patient suffering from an autoimmune disease. In these contexts, the disclosed molecules are powerful tools for monitoring the progress of a particular therapy. In addition to monitoring and quantifying antigen-specific T-cells, the disclosed molecules may also be used to purify such cells for adoptive immunotherapy. In one specific, non-limiting example, the disclosed human MHC molecules loaded with a tumor antigen may be used to purify tumor-antigen specific T-cells from a cancer patient. These cells may then be expanded in vitro before being returned to the patient as part of a cancer treatment. When conjugated with a toxic moiety, the two domain molecules may be used to kill T-cells having a particular antigen specificity. Alternatively, the molecules may also be used to induce anergy in such T-cells, or to induce suppressor T cells.

The two domain molecules may also be used in vivo to target specified antigen-specific T-cells. By way of example, a β1α1 molecule loaded with a portion of myelin basic protein (MBP) and administered to patients suffering from multiple sclerosis may be used to induce anergy in MBP-specific T-cells, or to induce suppressor T cells, thus alleviating the disease symptoms. Alternatively, such molecules may be conjugated with a toxic moiety to more directly kill the disease-causing T-cells.

These and other aspects of the disclosure are described in more detail in the following sections.

### BRIEF DESCRIPTION OF THE DRAWINGS

FIG. 1A shows the sequences of the prototypical β1∝1 cassette without an antigen coding region. Unique NcoI, PstI, and XhoI restriction sites are in bold. The end of the β1 domain and start of the al domain are indicated. FIG. 1B shows the sequence of an in-frame antigenic peptide/linker insertion sequence that can be incorporated into the expression cassette at the insertion site shown in FIG. 1A. This sequence includes the rat MBP-72-89 antigen, a flexible linker with an embedded thrombin cleavage site, and a unique SpeI restriction site that can be used for facile exchange of the antigen coding region. Example 2 below discusses the use of the equivalent peptide from Guinea pig, which has a serine in place of the threonine residue in the MBP-72-89 sequence. FIGS. 1C and 1D show exemplary Nco1/SpeI fragments that can be inserted into the expression cassette in place of the MBP-72-89 antigen coding region. FIG. 1C includes the MBP-55-69 antigen, FIG. 1D includes the CM-2 antigen.

FIGS. 2A and B show the structure-based design of the β1α1 molecule. A. Rat class II RT1.B, loaded with the encephalitogenic MBP-69-89 peptide (non-covalent association). B. The single-chain β1α1 molecule, loaded with MBP-69-89.

FIGS. 3A and B show direct detection of antigen-specific β1α1/polypeptide molecules binding rat T cells. The Al T cell hybridoma (BV8S2 TCR+) and the CM-2 cell line (BV8S2 TCR-) were incubated 17 hours at 4 C with various β1α1 constructs, washed, stained for 15 min with OX6-PE (α-RT1.B) or a PE-isotype control and then analyzed by FACS. Background expression of I-A on the CM-2 line was blocked with unlabeled OX-6. A. Histogram showing staining of the A1 hybridoma. B. Histogram showing staining of the CM-2 cell line.

FIG. 4 is a graph showing binding of A488 conjugated β1α1/polypeptide molecules to rat BV8S2 TCR. β1α1 molecules were conjugated with Alexa-488 dye, loaded with MBP-69-89, incubated with the A1 T cell hybridomas (BV8S2 TCR+) for 3 hours at 4C and then analyzed by FACS. A488-β1α1(empty) and A488-β1α1/MBP-69-89, as indicated.

FIG. 5 is a bar graph showing that the β1α1/MBP-69-89 complex blocks antigen specific proliferation in an IL-2 reversible manner. Short-term T cell lines selected with MBP-69-89 peptide from lymph node cells from rats immunized 12 days earlier with Gp-MBP/CFA were pre-treated for 24 hours with β1α1 constructs, washed, and then used in proliferation assays in which the cells were cultured with and without 20 Units/ml IL-2. Cells were incubated for three days, the last 18 hr in the presence of [³H]thymidine (0.5 μCi/10 μl/well). Values indicated are the mean CPM±SEM. Background was 210 CPM. Column a. Control proliferation assay without IL-2. Column b. 20 μM β1α1/MBP-55-69 pretreatment. Column c. 10 nM β1α1/MBP-69-89 pretreatment. Column d. 10 nM β1α1/MBP-69-89 plus IL-2 during the proliferation assay. A single representative experiment is shown; the experiment was done twice. *indicates significant (p<0.001) inhibition with β1α1/MBP-69-89 versus control cultures.

FIGS. 6A-D are graphs showing clinical protection from experimental autoimmune encephalomyelitis with the β1α1/MBP-69-89 complex. Groups of Lewis rats (n=6) were injected with 25 μg of Gp-MBP/CFA to induce clinical EAE. On days 3, 7, 9, 11, and 14 after disease induction rats were given β1α1/peptide complex, peptide alone, or were left untreated, as indicated. A. No treatment, or 2 μg MBP-69-89 peptide alone, as indicated. B. 300 μg of β1α1/(empty) complex in saline. C. 300 μg of β1α1/CM-2 complex in saline. D. 30 μg of β1α1/MBP-69-89 complex in saline. Daily body weight (grams, right-hand y-axis) is plotted for the 300 μg β1α1/peptide complex treatments. A single representative experiment is shown; the experiment was done three times. Values indicate mean clinical score ±SEM on each day of clinical disease. 30 μg of complex is equivalent to 2 μg of free peptide.

FIG. 7 is a graph showing treatment of established EAE with β1α1/MBP-69-89 complex. Groups of Lewis rats (n=6) were injected with 25 μg of Gp-MBP/CFA to induce clinical EAE. On the day of onset of clinical signs (day 11), day 13, and day 15, rats were given 300 μg of β1α1/MBP-69-89 complex (indicated by arrows) or were left untreated. A single representative experiment is shown; the experiment was done twice. Values indicate mean clinical score ±SEM on each day of clinical disease.

FIGS. 8A and B are graphs showing that the β1α1/MBP-69-89 complex specifically inhibits the DTH response to MBP 69-89. A. Change in ear thickness 24 hrs after challenge with PPD. B. Change in ear thickness 24 hrs after challenge with MBP-69-89. Values indicate mean score ±SEM. *Indicates significant difference between control and treated (p=0.01). A single representative experiment is shown; the experiment was done twice.

FIG. 9 is a graph showing that T cell responses to MBP-69-89 were inhibited in Lewis rats treated with 300 μg β1α1/MBP-69-89 complex. Lymph node cells were collected from control and treated rats after recovery of controls from EAE (day 17) and stimulated with optimal concentrations of Gp-MBP, Gp-MBP-69-89 peptide, or PPD. *Indicates significant difference between control and treated (*p<0.05; **p<0.001). Note inhibition with Gp MBP and MBP-69-89 peptide but not to PPD in treated rats.

FIGS. 10A-C shows the amino acid sequences of exemplary (A) human (DRA and DRB1 0101), (B) mouse (I-EK) and (C) rat (RT1.B) 13 and β1 domains (the initiating methione and glycine sequences in the rat sequence were included in a construct for translation initiation reasons).

FIG. 11 shows the amino acid sequences of exemplary α1 and α2 domains derived from human MHC class I B*5301.

FIG. 12 shows schematic models of human HLA-DR2-derived recombinant TCR ligands (RTLs). FIG. 12(a) is a schematic scale model of an MHC class II molecule on the surface of an APC. The polypeptide backbone extra-cellular domain is based on the crystallographic coordinates of HLA-DR2 (PDB accession code 1BX2) (19). The transmembrane domains are shown schematically as 0.5 nm cylinders, roughly the diameter of a poly-glycine alpha-helix. The α1, α2, β1 and β2 domains are labeled, as well as the carboxyl termini of the MHC class II heterodimers. FIG. 12(b) is a schematic of the RTL303 molecule containing covalently linked β1 and α1 domains from HLA-DR2 and covalently coupled MBβ85-99 peptide. The view of the RTLs is symmetry-related to the MHC class II molecule in panel (a) by rotation around the long-axis of bound peptide by ˜45° (y-axis) and ˜45° (Z-axis). Top, the same shading scheme as in panel (a), with primary TCR contact residues H11, F12, K14 and N15 labelled (39). Middle, shaded according to electrostatic potential (EP). The shading ramp for EP ranges from dark (most positive) to light (most negative) (40). Bottom, shaded according to lipophilic potential (LP). The shading ramp for LP ranges from dark(most lipophilic area of the molecule) to light (most hydrophilic area) (41).

FIG. 13 is the nucleotide and protein sequence of human HLA-DR2-derived RTL303 (SEQ ID NO: 40 and 41, respectively). RTL303 was derived from sequences encoding the beta-1 and alpha-1 domains of HLA-DR2 (human DRBI * 1501/DRA*0101) and sequence encoding the human MBβ85-99 peptide. Unique NcoI, Spel and XhoI restriction sites are in bold. The end of the beta-1 domain and start of the alpha-1 domain are indicated by an arrow (▾). RTL303 contains an in-frame peptide/linker insertion encoding the human MBβ85-99 peptide (bold), a flexible linker with an embedded thrombin cleavage site (23), and a unique Spel restriction site which can be used for rapidly exchanging the encoded amino-terminal peptide. RTL301 is identical to RTL303 except for a single point mutation resulting in an F150L substitution. Two additional proteins used in this study, RTL300 and RTL302, are “empty” versions of RTL301 and RTL303, respectively. These molecules lack the peptide/linker insertion (residues 16-115). Codon usage for glycines 32, and 51 have been changed from the native sequence for increased levels of protein expression in E. coli (G. G. Burrows, unpublished observations).

FIG. 14 shows the purification of human HLA-DR2-derived RTL303. FIG. 14(A) is the ion exchange FPLC of RTL303. Insert left: Mr, molecular weight standards; U, uninduced cells; I, induced cells, showing high-level expression of RTL303. Insert Right: Fractions 25-28 contain partially purified RTL303. FIG. 14(B) is size-exclusion chromatography of RTL303. Insert: fractions 41-44, containing purified RTL303; Mr, molecular weight standards; Red, reduced RTL303; NR, non-reduced RTL303.

FIG. 15 is a digital image of a Western blot demonstrating purified and refolded DR2-derived RTLs have a native disulfide bond. Samples of RTLs were boiled for 5 minutes in Laemmli sample buffer with or without the reducing agent β-mercaptoethanol (β-ME), and then analyzed by SDS-PAGE (12%). Non-reduced RTLs (−lane) have a smaller apparent molecular weight than reduced RTLs (+lane), indicating the presence of a disulfide bond. First and last lanes show the molecular weight standards carbonic anhydrase (31 kD) and soybean trypsin inhibitor (21.5kD). RTLs (+/−β-ME), as indicated.

FIG. 16 is a digital image demonstrating circular dichroism shows the DR2-derived RTLs have highly ordered structures. CD measurements were performed at 20° C. on a Jasco J-500 instrument using 0.1 mm cells from 260 to 180 nm. Concentration values for each protein solution were determined by amino acid analysis. Buffer, 50 mM potassium phosphate, 50 mM sodium fluoride, pH 7.8. Analysis of the secondary structure was performed using the variable selection method (42).

FIG. 17 is a graph of experiments that demonstrate that thermal denaturation shows a high degree of cooperativity and stability of the DR2-derived RTLs. CD spectra were monitored at 222 mun as a function of temperature. The heating rate was 10° C./hr. The graph charts the percent of unfolding as a function of temperature. 1.0 corresponds to the completely unfolded structure.

FIG. 18 is a schematic diagram showing interactions of atoms within 4A of residue F150. Distances were calculated using coordinates from 1BX2 (19). Inset; RTL303 showing the location of residue F 150 within the molecule.

FIG. 19 shows the structure-based design of the human HLA-DR2-derived Recombinant TCR ligands (RTLs). FIG. 19A is a schematic scale model of an MHC class II molecule on the surface of an APC. The polypeptide backbone extracellular domain is based on the crystallographic coordinates of HLA-DRI (PDB accession code 1AQD) (17). The transmembrane domains are shown schematically as 0.5 nm cylinders, roughly the diameter of a poly-glycine alpha-helix. The carboxyl termini of the MHC class II heterodimers are labeled. FIG. 19B is a diagram of the HLA-DR2 β1α1-derived RTL303 molecule containing covalently coupled MBβ85-99 peptide.

FIG. 19C is a diagram of the HLA-DR2 β1α1-derived RTL311 molecule containing covalently coupled C-ABL peptide. The view of the RTLs is symmetry-related to the MHC class II molecule in panel (a) by rotation around the long-axis of bound peptide by ˜45° (y-axis) and ˜45° (Z-axis). Left, the same shading scheme as in panel (A), with primary TCR contact residues labelled. Middle, shaded according to electrostatic potential (EP). The shading ramp for EP ranges from dark (most positive) to light (most negative) (20). Right, shaded according to lipophilic potential (LP). The shading ramp for LP ranges from dark (highest lipophilic area of the molecule) to light (highest hydrophilic area) (21). The program Sybyl (Tripos Associates, St. Louis, Mo.) was used to generate graphic images using an O2 workstation (Silicon Graphics, Mountain View, Calif.) and coordinates deposited in the Brookhaven Protein Data Bank (Brookhaven National Laboratories, Upton, N.Y.). Structure-based homology modeling of RTLs was based on the refined crystallographic coordinates of HLA-DR2 complexed with MBP peptide (DRA*0101, DRB* 1501) (48). Amino acid residues in the HLA-DR2 MBP peptide complex (PDB accession number 1BX2) were substituted with the CABL side chains, with the peptide backbone of HLA-DR2 modeled as a rigid body during structural refinement using local energy minimization.

FIG. 20 is a series of bar graphs showing the response of T cell clones. DR2 restricted T cell clones MR#3-1, specific for MBP-85-99 peptide, and MR#2-87, specific for CABL-b3α2 peptide, and a DR7 restricted T cell clone CP#1-15 specific for MBP-85-99 peptide were cultured at 50,000 cells/well with medium alone or irradiated (2500 rad) frozen autologous PBMC (150,000/well) plus peptide-Ag (MBP-85-99 or CABL, 10 μg/ml) in triplicate wells for 72 hr, with 3H-thymidine incorporation for the last 18 hr. Each experiment shown is representative of at least two independent experiments. Bars represent CPM ±SEM.

FIG. 21 is a graph showing that zeta chain phosphorylation induced by RTL treatment is Ag-specific. DR2 restricted T cell clones MR#3-1 specific for MBP-85-99 peptide or MR#2-87 specific for CABL-b3α2 peptide, were incubated at 37° C. with medium alone (control), or with 20 μM RTL303 or RTL311. Western blot analysis of phosphorylated ζ (zeta) shows a pair of phospho-protein species of 21 and 23 kD, termed p21 and p23, respectively. Quantification of the bands showed a distinct change in the p21/p23 ratio that peaked at 10 minutes. Each experiment shown is representative of at least three independent experiments. Points represent mean ±SEM.

FIG. 22 shows the fluorescence emssion ratio of T cells stimulated with RTLs. RTLs induce a sustained high Calcium signal in T cells. Calcium levels in the DR2 restricted T cell clone MR#3-1 specific for the MBP-85-99 peptide were monitored by single cell analysis. RTL303 treatment induced a sustained high calcium signal, whereas RTL301 (identical to RTL303 except a single point mutation, F150L) showed no increase in calcium signal over the same time period. The data is representative of two separate experiments with at least 14 individual cells monitored in each experiment.

FIG. 23 is a set of bar graphs showing ERK activity is decreased in RTL treated T cells. DR2 restricted T cell clone MR#3-1 specific for the MBP-85-99 peptide or MR#2-87 specific for CABL b3α2 peptide were incubated for 15 min at 37° C. with no addition (control), and with 20 or 8 AM RTL303 or RTL311. At the end of the 15-min incubation period, cells were assayed for activated, phosphorylated ERK (P-ERK) and total ERK (T-ERK). Quantification of activated P-ERK is presented as the fraction of the total in control (untreated) cells. Each experiment shown is representative of at least three independent experiments. Bars represent mean ±SEM.

FIG. 24 is a series of graphs showing direct antigen-specific modulation of IL-10 cytokine production in T cell clones was induced by RTL treatment. DR2 restricted T cell clones MR#3-1 and MR#2-87 were cultured in medium alone (-control), anti-CD3 mAb, 20 AM RTL303 or RTL3 11 for 72 hours. Proliferation was assessed by ³H-thymidine uptake. Cytokines (pg/ml) profiles were monitored by immunoassay (ELISA) of supernatants. Each experiment shown was representative of at least three independent experiments. Bars represent mean ±SEM. Clone MR#3-1 showed initial proliferation to anti-CD3, but not to RTLs.

FIG. 25 is a set of graphs showing IL-10 cytokine production induced by RTL pre-treatment was maintained after stimulation with APC/peptide. T cells showed a reduced ability to proliferate and produce cytokines after anti-CD3 or RTL treatment, and the RTL effect was antigen and MHC specific. IL-10 was induced only by specific RTLs, and Il-10 production was maintained even after restimulation with APC/antigen. T cell clones were cultured at 50,000 cells/well with medium, anti-CD3, or 20 μM RTLs in triplicate for 48 hours, and washed once with RPMI. After the wash, irradiated (2500 rad) frozen autologous PBMC (150,000/well) plus peptide-Ag (MBP-85-99 at 10 μg/ml) were added and the cells incubated for 72 hr with ³H-thymidine added for the last 18 hr. Each experiment shown is representative of at least two independent experiments. Bars represent mean ±SEM. For cytokine assays, clones were cultured with 10 pg/ml anti-CD3 or 20 1M RTL303 or RTL311 for 48 hours, followed by washing with RPMI and re-stimulation with irradiated autologous PBMC (2500 rad, T:APC=1:4) plus peptide-Ag (10 μg/ml) for 72 hours. Cytokines (pg/ml) profiles were monitored by immunoassay (ELISA) of supernatants. Each experiment shown is representative of at least three independent experiments. Bars represent mean ±SEM.

### SEQUENCE LISTING

The sequence listing appended hereto includes sequences as follows:

SEQ ID NO: 1: the nucleic acid of a single chain β1α1 expression cassette.

SEQ ID NO:2: the amino acid sequence encoded by the construct shown in SEQ ID NO:1.

SEQ ID NO:3: the nucleic acid sequence of an antigen/linker insert suitable for insertion into the expression cassette shown in SEQ ID NO:1.

SEQ ID NO:4: the amino acid sequence encoded by the sequence shown in SEQ ID NO:3.

SEQ ID NOS:5 and 7: alternative antigen encoding sequences for the expression cassette and, SEQ ID NOS:6 and 8, the antigen sequences encoded by the sequences shown in SEQ ID NOS:5 and 7, respectively.

SEQ ID NOS:9-20 and 28-29 show PCR primers use to amplify components of the 1 1 expression cassette.

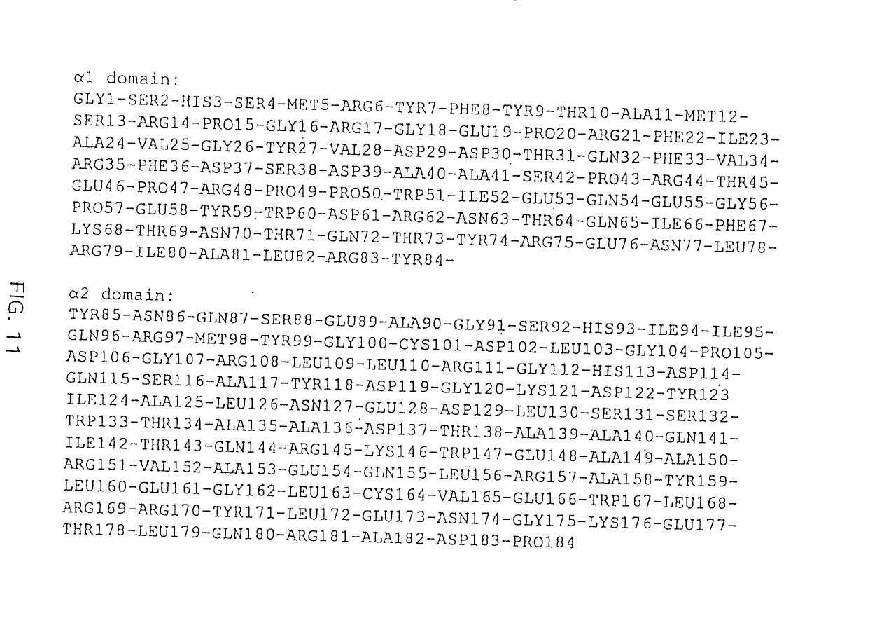

SEQ ID NO:21 shows the exemplary al and 0:2 domains depicted in FIG. 11.

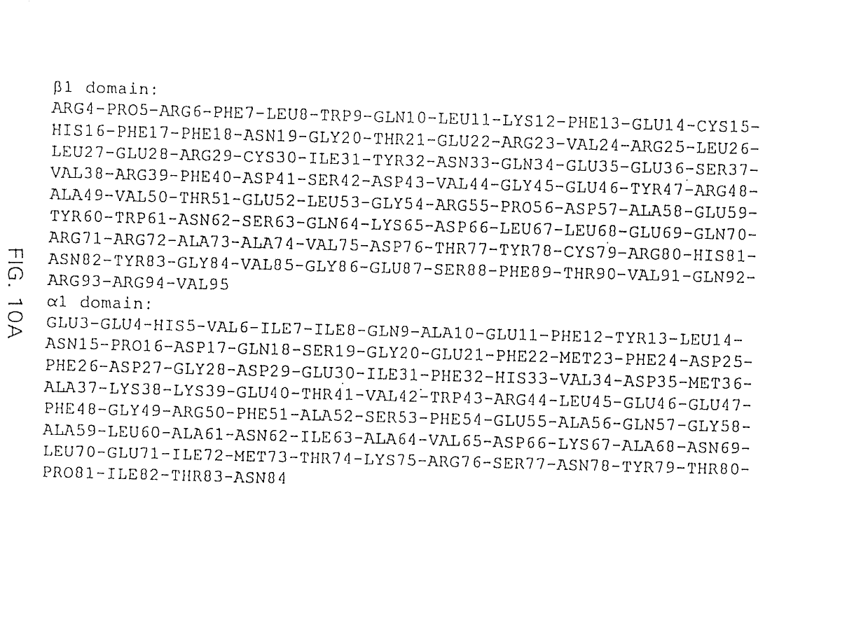

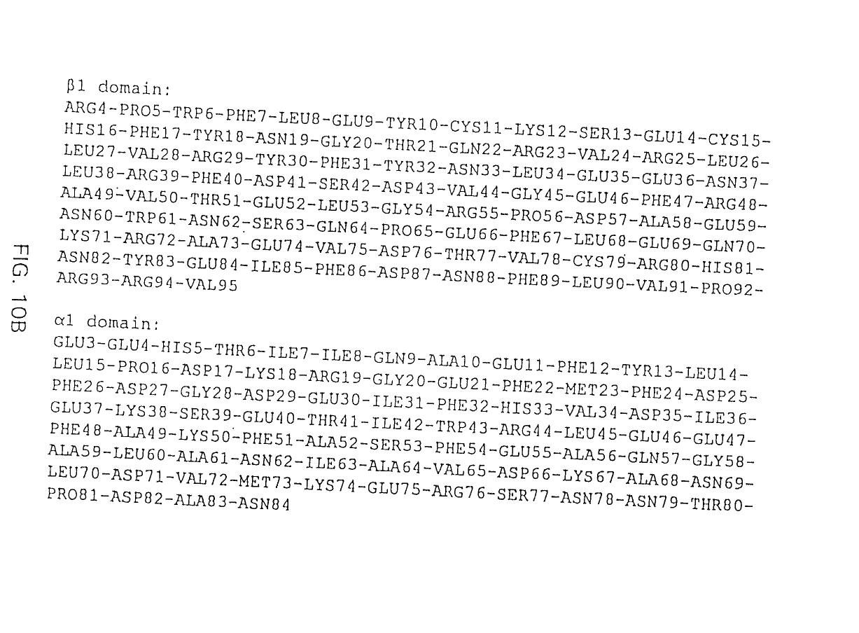

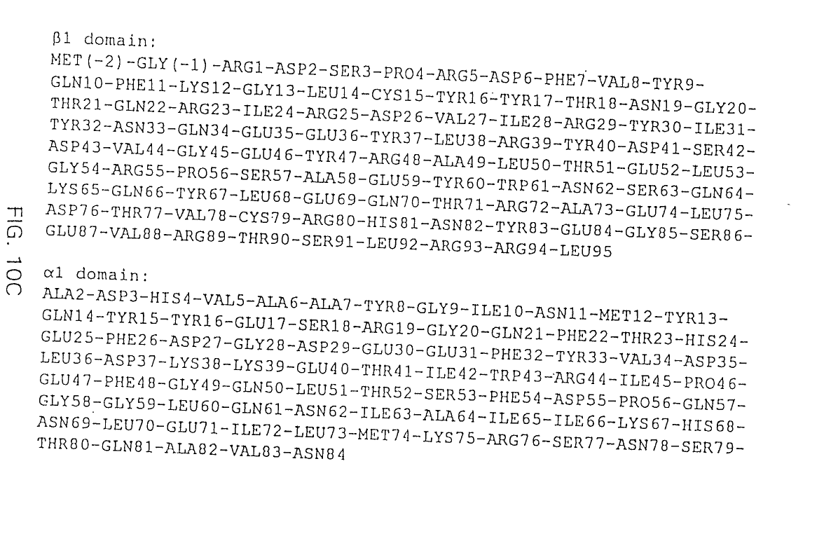

SEQ ID NOS:22-24 show the exemplary β1 and α1 domains depicted in FIG. 10.

SEQ ID NOS:25-27 and 30 show peptides sequences used in various aspects of the invention.

SEQ ID NO:28-31 are the nucleic acid sequence of primers used for human β1∝1.

SEQ ID NO:32-33 are the nucleic acid sequence of primers for T7.

SEQ ID NO:34-35 are the nucleic acid sequence of primers for myelin basic protein.

SEQ ID NO:36-37 are primers for human BA-F150L.

SEQ ID NO:38 is the amino acid sequence of the MBP 85-89 peptide.

SEQ ID NO:39 is the amino acid sequence of the BCR-ABL b3a2 peptide.

### DETAILED DESCRIPTION OF THE INVENTION

1. Definitions

In order to facilitate review of the various embodiments of the invention, the following definitions of terms and explanations of abbreviations are provided:

β1α1 polypeptide: A recombinant polypeptide comprising the α1 and β1 domains of a MHC class II molecule in covalent linkage. To ensure appropriate conformation, the orientation of such a polypeptide is such that the carboxy terminus of the β1 domain is covalently linked to the amino terminus of the α1 domain. In one embodiment, the polypeptide is a human β1α1 polypeptide, and includes the α1 and β1 domains for a human MHC class II molecule. One specific, non-limiting example of a human β1α1 polypeptide is a molecule wherein the carboxy terminus of the β1 domain is covalently linked to the amino terminus of the α1 domain of an HLA-DR molecule. Additional specific non-limiting examples of a human β1α1 polypeptide is a molecule wherein the carboxy terminus of the β1 domain is covalently linked to the amino terminus of the alI domain of an a HLA-DR(either A or B), a HLA-DP(A and B), or a HLA-DQ(A and B) molecule. In one embodiment, the β1α1 polypeptide does not include a β2 domain. In another embodiment, the β1α1 polypeptide does not include an α2. In yet another embodiment, the β1α1 polypeptide does not include either an α2 or a β2 domain.

β1α1 gene: A recombinant nucleic acid sequence including a promoter region operably linked to a nucleic acid sequence encoding a β1α1 polypeptide. In one embodiment the β1α1 polypeptide is a human β1α1 polypeptide.

α1α2 polypeptide: A polypeptide comprising the α1 and α2 domains of a MHC class I molecule in covalent linkage. The orientation of such a polypeptide is such that the carboxy terminus of the α1 domain is covalently linked to the amino terminus of the α2 domain. An α1α2 polypeptide comprises less than the whole class I α chain, and usually omits most or all of the α3 domain of the α chain. Specific non-limiting examples of an α1α2 polypeptide are polypeptides wherein the carboxy terminus of the α1 domain is covalently linked to the amino terminus of the α2 domain of an HLA-A, -B or -C molecule. In one embodiment, the α3 domain is omitted from an α1α2 polypeptide, thus the α1α2 polypeptide does not include an α3 domain.

α1α2 gene: A recombinant nucleic acid sequence including a promoter region operably linked to a nucleic acid sequence encoding an α1α2 polypeptide.

Antigen (Ag): A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. The term “antigen” includes all related antigenic epitopes and antigenic determinants.

Autoimmune disorder: A disorder in which the immune system produces an immune response (e.g. a B cell or a T cell response) against an endogenous antigen, with consequent injury to tissues.

CD8+ T cell mediated immunity: An immune response implemented by presentation of antigens to CD8+ T cells.

cDNA (complementary DNA): A piece of DNA lacking internal, non-coding segments (introns) and regulatory sequences that determine transcription. cDNA is synthesized in the laboratory by reverse transcription from messenger RNA extracted from cells.

Cytokine: Proteins made by cells that affect the behavior of other cells, such as lymphocytes. In one embodiment, a cytokine is a chemokine, a molecule that affects cellular trafficking.

Domain: A domain of a polypeptide or protein is a discrete part of an amino acid sequence that can be equated with a particular function. For example, the a and r polypeptides that constitute a MHC class II molecule are each recognized as having two domains, α1, α2 and β1, β2, respectively. Similarly, the α chain of MHC class I molecules is recognized as having three domains, α1, α2 and α3. The various domains in each of these molecules are typically joined by linking amino acid sequences. In one embodiment, when selecting the sequence of a particular domain for inclusion in a recombinant molecule, the entire domain is included; to ensure that this is done, the domain sequence may be extended to include part of the linker, or even part of the adjacent domain. For example, when selecting the al domain of HLA-DR A, the selected sequence will generally extend from amino acid residue number 1 of the α chain, through the entire α1 domain and will include all or part of the linker sequence located at about amino acid residues 76-90 (at the carboxy terminus of the α1 domain, between the α1 and α2 domains).

However, the precise number of amino acids in the various MHC molecule domains varies depending on the species of mammal, as well as between classes of genes within a species. Rather than a precise structural definition based on the number of amino acids, it is the maintenance of domain function that is important when selecting the amino acid sequence of a particular domain. Moreover, one of skill in the art will appreciate that domain function may also be maintained if somewhat less than the entire amino acid sequence of the selected domain is utilized. For example, a number of amino acids at either the amino or carboxy terminii of the α1 domain may be omitted without affecting domain function. Typically however, the number of amino acids omitted from either terminus of the domain sequence will be no greater than 10, and more typically no greater than 5. The functional activity of a particular selected domain may be assessed in the context of the two-domain MHC polypeptides provided by this invention (i.e., the class II β1α1 or class I α1α2 polypeptides) using the antigen-specific T-cell proliferation assay as described in detail below. For example, to test a particular β1 domain, it will be linked to a functional α1 domain so as to produce a β1α1 molecule and then tested in the described assay. A biologically active β1α1 or α1α2 polypeptide will inhibit antigen-specific T cell proliferation by at least about 50%, thus indicating that the component domains are functional. Typically, such polypeptides will inhibit T-cell proliferation in this assay system by at least 75% and sometimes by greater than about 90%.

Epitope: An antigenic determinant. These are particular chemical groups or peptide sequences on a molecule that are antigenic, i.e. that elicit a specific immune response. An antibody binds a particular antigenic epitope.

Functionally Equivalent: Sequence alterations, in either an antigen epitope or a β1α1, or an α1α2 peptide, that yield the same results as described herein. Such sequence alterations can include, but are not limited to, conservative substitutions, deletions, mutations, frameshifts, and insertions.

IL-10: A cytokine that is a homodimeric protein with subunits having a length of 160 amino acids. Human IL-10 shows 73 percent amino acid homology with murine IL-1 0. The human IL-10 gene contains four exons and maps to chromosome 1 (for review see de Waal-Malefyt R et al., Curr. Opin. Immunology 4: 314-20, 1992; Howard and O'Garra, Immunology Today 13: 198-200, 1992; Howard et al., J. Clin. Immunol. 12: 239-47, 1992).

IL-10 is produced by murine T-cells (Th2 cells but not Th1 cells) following their stimulation by lectins. In humans, IL-10 is produced by activated CD 8+ peripheral blood T-cells, by Th0, Th1-, and Th2-like CD4+ T-cell clones after both antigen-specific and polyclonal activation, by B-cell lymphomas, and by LPS-activated monocytes and mast cells. B-cell lines derived from patients with acquired immunodeficiency syndrome and Burkitt's lymphoma constitutively secrete large quantities of IL 10.

IL-10 has a variety of biological functions. For example, IL-10 inhibits the synthesis of a number of cytokines such as IFN-γ, IL-2 and TNF-α in Th1 subpopulations of T-cells but not of Th2 cells. This activity is antagonized by IL-4. The inhibitory effect on IFN-γ production is indirect and appears to be the result of a suppression of IL-12 synthesis by accessory cells. In the human system, IL-10 is produced by, and down-regulates the function of, Th1 and Th2 cells. IL-10 is also known to inhibit the synthesis of IL-1, IL-6, and TNF-α by promoting, among other things, the degradation of cytokine mRNA. Expression of IL-10 can also lead to an inhibition of antigen presentation. In human monocytes, IFN-γ and IL-10 antagonize each other's production and function. In addition, IL-10 has been shown also to be a physiologic antagonist of IL-12. IL-10 also inhibits mitogen- or anti-CD3-induced proliferation of T-cells in the presence of accessory cells and reduces the production of IFN-γ and IL-2. IL-10 appears to be responsible for most or all of the ability of Th2 supernatants to inhibit cytokine synthesis by Th1 cells.

IL-10 can be detected with a sensitive ELISA assay. In addition, the murine mast cell line D36 can be used to bioassay human IL-10. Flow cytometry methods have also been used to detect IL-10 (see Abrams et al. Immunol. Reviews 127: 5-24, 1992; Fiorentino et al., J. Immunol. 147: 3815-22, 1991; Kreft et al, J. Immunol. Methods 156: 125-8, 1992; Mosmann et al., J. Immunol. 145: 2938-45, 1990), see also the Examples section below.

Immune response: A response of a cell of the immune system, such as a B cell, or a T cell, to a stimulus. In one embodiment, the response is specific for a particular antigen (an “antigen-specific response”). In one embodiment, an immune response is a T cell response, such as a Th1, Th2, or Th3 response. In another embodiment, an immune response is a response of a suppressor T cell.

Isolated: An “isolated” nucleic acid has been substantially separated or purified away from other nucleic acid sequences in the cell of the organism in which the nucleic acid naturally occurs, i.e., other chromosomal and extrachromosomal DNA and RNA. The term “isolated” thus encompasses nucleic acids purified by standard nucleic acid purification methods. The term also embraces nucleic acids prepared by recombinant expression in a host cell as well as chemically synthesized nucleic acids.

Linker sequence: A linker sequence is an amino acid sequence that covalently links two polypeptide domains. Linker sequences may be included in the recombinant MHC polypeptides of the present invention to provide rotational freedom to the linked polypeptide domains and thereby to promote proper domain folding and inter- and intra-domain bonding. By way of example, in a recombinant polypeptide comprising Ag-β1-α1 (where Ag=antigen) linker sequences may be provided between both the Ag and β1 domains and between β1 and α1 domains. Linker sequences, which are generally between 2 and 25 amino acids in length, are well known in the art and include, but are not limited to, the glycine(4)-serine spacer (GGGGS x3) described by Chaudhary et al. (1989). Other linker sequences are described in the Examples section below.

Recombinant MHC class I α1α2 polypeptides according to the present invention include a covalent linkage joining the carboxy terminus of the α1 domain to the amino terminus of the α2 domain. The α1 and α2 domains of native MHC class I α chains are typically covalently linked in this orientation by an amino acid linker sequence. This native linker sequence may be maintained in the recombinant constructs; alternatively, a recombinant linker sequence may be introduced between the α1 and α2 domains (either in place of or in addition to the native linker sequence).

Mammal: This term includes both human and non-human mammals. Similarly, the term “patient” or “subject” includes both human and veterinary subjects.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter effects the transcription or expression of the coding sequence. Generally, operably linked DNA sequences are contiguous and, where necessary to join two protein coding regions, the open reading frames are aligned.

ORF (open reading frame): A series of nucleotide triplets (codons) coding for amino acids without any termination codons. These sequences are usually translatable into a polypeptide.

Pharmaceutical agent or drug: A chemical compound or composition capable of inducing a desired therapeutic or prophylactic effect when properly administered to a subject.

Pharmaceutically acceptable carriers: The pharmaceutically acceptable carriers useful with the polypeptides and nucleic acids described herein are conventional. Remington 's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing or treating a disease: “Preventing” a disease refers to inhibiting the full development of a disease, for example in a person who is known to have a predisposition to a disease such as an autoimmune disorder. An example of a person with a known predisposition is someone with a history of diabetes in the family, or who has been exposed to factors that predispose the subject to a condition, such as lupus or rheumatoid arthritis. “Treatment” refers to a therapeutic intervention that ameliorates a sign or symptom of a disease or pathological condition after it has begun to develop.

Probes and primers: Nucleic acid probes and primers may readily be prepared based on the nucleic acids provided by this invention. A probe comprises an isolated nucleic acid attached to a detectable label or reporter molecule. Typical labels include radioactive isotopes, ligands, chemiluminescent agents, and enzymes. Methods for labeling and guidance in the choice of labels appropriate for various purposes are discussed, e.g., in Sambrook et al. (1989) and Ausubel et al. (1987).

Primers are short nucleic acids, preferably DNA oligonucleotides 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Methods for preparing and using probes and primers are described, for example, in Sambrook et al. (1989), Ausubel et al. (1987), and Innis et al., (1990). PCR primer pairs can be derived from a known sequence, for example, by using computer programs intended for that purpose such as Primer (Version 0.5, ©1991, Whitehead Institute for Biomedical Research, Cambridge, Mass.).

Purified: The term purified does not require absolute purity; rather, it is intended as a relative term. Thus, for example, a purified recombinant MHC protein preparation is one in which the recombinant MHC protein is more pure than the protein in its originating environment within a cell. A preparation of a recombinant MHC protein is typically purified such that the recombinant MHC protein represents at least 50% of the total protein content of the preparation. However, more highly purified preparations may be required for certain applications. For example, for such applications, preparations in which the MHC protein comprises at least 75% or at least 90% of the total protein content may be employed.

Recombinant: A recombinant nucleic acid or polypeptide is one that has a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Variants of MHC domain polypeptides will possess a relatively high degree of sequence identity when aligned using standard methods. (An “MHC domain polypeptide” refers to a β1 or an al domain of an MHC class II polypeptide or an al or an α2 domain of an MHC class I polypeptide).

Methods of alignment of sequences for comparison are well known in the art. Altschul et al. (1994) presents a detailed consideration of sequence alignment methods and homology calculations. The NCBI Basic Local Alignment Search Tool (BLAST) (Altschul et al., 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the NCBI website. A description of how to determine sequence identity using this program is available at the NCBI website, as are the default parameters.

Variants of MHC domain polypeptides are typically characterized by possession of at least 50% sequence identity counted over the full length alignment with the amino acid sequence of a native MHC domain polypeptide using the NCBI Blast 2.0, gapped blastp set to default parameters. Proteins with even greater similarity to the reference sequences will show increasing percentage identities when assessed by this method, such as at least 60%, at least 65%, at least 70%, at least 75%, at least 80%, at least 90% or at least 95% sequence identity. When less than the entire sequence is being compared for sequence identity, variants will typically possess at least 75% sequence identity over short windows of 10-20 amino acids, and may possess sequence identities of at least 85% or at least 90% or 95% depending on their similarity to the reference sequence. Methods for determining sequence identity over such short windows are described at the NCBI website. Variants of MHC domain polypeptides also retain the biological activity of the native polypeptide. For the purposes of this invention, that activity is conveniently assessed by incorporating the variant domain in the appropriate β1α1 or α1α2 polypeptide and determining the ability of the resulting polypeptide to inhibit antigen specific T-cell proliferation in vitro, or to induce T suppressor cells or the expression of IL-10 as described in detail below.

Therapeutically effective dose: A dose sufficient to prevent advancement, or to cause regression of the disease, or which is capable of relieving symptoms caused by the disease, such as pain or swelling.

Tolerance: Diminished or absent capacity to make a specific immune response to an antigen. Tolerance is often produced as a result of contact with an antigen in the presence of a two domain MHC molecule, as described herein. In one embodiment, a B cell response is reduced or does not occur. In another embodiment, a T cell response is reduced or does not occur. Alternatively, both a T cell and a B cell response can be reduced or not occur.

Transduced and Transformed: A virus or vector “transduces” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Vector: A nucleic acid molecule as introduced into a host cell, thereby producing a transformed host cell. A vector may include nucleic acid sequences that permit it to replicate in the host cell, such as an origin of replication. A vector may also include one or more selectable marker genes and other genetic elements known in the art. The term “vector” includes viral vectors, such as adenoviruses, adeno-associated viruses, vaccinia, and retroviruses vectors.

Additional definitions of terms commonly used in molecular genetics can be found in Benjamin Lewin, Genes V published by Oxford University Press, 1994 (ISBN 0-19-854287-9); Kendrew et al (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The following sections provide detailed guidance on the design, expression and uses of the recombinant MHC molecules of the invention. Unless otherwise stated, standard molecular biology, biochemistry and immunology methods are used in the present invention unless otherwise described. Such standard methods are described in Sambrook et al. (1989), Ausubel et al (1987), Innis et al. (1990) and Harlow and Lane (1988). The following U.S. patents which relate to conventional formulations of MHC molecules and their uses are incorporated herein by reference to provide additional background and technical information relevant to the present invention: U.S. Pat. Nos. 5,130,297; 5,194,425; 5,260,422; 5,284,935; 5,468,481; 5,595,881; 5,635,363; 5,734,023.

2. Design of Recombinant MHC Class II β1α1Molecules

The amino acid sequences of mammalian MHC class II α and β chain proteins, as well as nucleic acids encoding these proteins, are well known in the art and available from numerous sources including GenBank. Exemplary sequences are provided in Auffray et al. (1984) (human HLA DQ α); Larhammar et al. (1983) (human HLA DQ β); Das et al. (1983) (human HLA DR α); Tonnelle et al. (1985) (human HLA DR β); Lawrance et al. (1985) (human HLA DP α); Kelly et al. (1985) (human HLA DP β); Syha et al. (1989) (rat RT1.B α); Syha-Jedelhauser et al. (1991) (rat RT1.B β); Benoist et al. (1983) (mouse I-A α); Estess et al. (1986) (mouse I-A β), all of which are incorporated by reference herein. In one embodiment, the MHC class II protein is a human MHC class II protein.

The recombinant MHC class II molecules of the present invention comprise the β1 domain of the MHC class II β chain covalently linked to the α1 domain of the MHC class II cc chain. The α1 and β1 domains are well defined in mammalian MHC class II proteins. Typically, the α1 domain is regarded as comprising about residues 1-90 of the mature chain. The native peptide linker region between the α1 and α2 domains of the MHC class II protein spans from about amino acid 76 to about amino acid 93 of the α chain, depending on the particular cc chain under consideration. Thus, an α1 domain may include about amino acid residues 1-90 of the α chain, but one of skill in the art will recognize that the C-terminal cut-off of this domain is not necessarily precisely defined, and, for example, might occur at any point between amino acid residues 70-100 of the α chain. The composition of the al domain may also vary outside of these parameters depending on the mammalian species and the particular a chain in question. One of skill in the art will appreciate that the precise numerical parameters of the amino acid sequence are much less important than the maintenance of domain function.

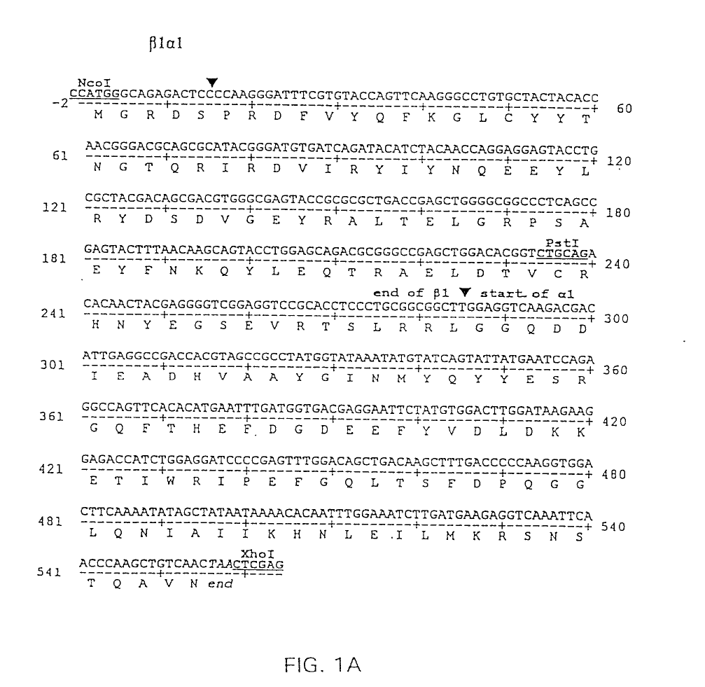

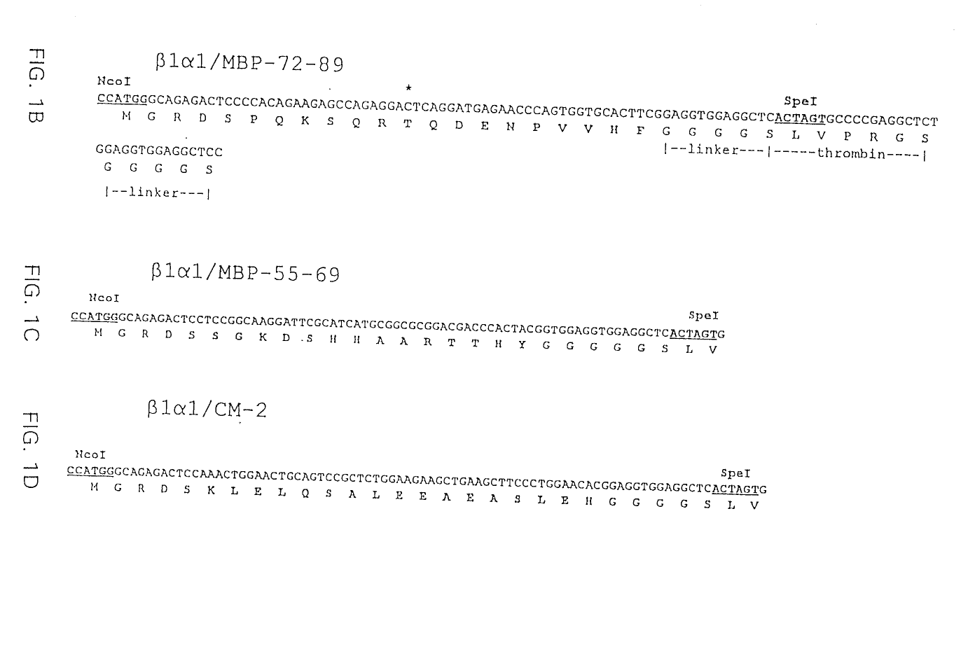

Similarly, the β1 domain is typically regarded as comprising about residues 1-90 of the mature β chain. The linker region between the al and α2 domains of the MHC class II protein spans from about amino acid 85 to about amino acid 100 of the α chain, depending on the particular o. chain under consideration. Thus, the al protein may include about amino acid residues 1-100, but one of skill in the art will again recognize that the C-terminal cut-off of this domain is not necessarily precisely defined, and, for example, might occur at any point between amino acid residues 75-105 of the α chain. The composition of the al domain may also vary outside of these parameters depending on the mammalian species and the particular ox chain in question. Again, one of skill in the art will appreciate that the precise numerical parameters of the amino acid sequence are much less important than the maintenance of domain function. Exemplary β1α1 molecules from human, rat and mouse are depicted in FIG. 1. In one embodiment, the β1α1 molecules do not include a β2 domain. In another embodiment, the β1α1 molecules do not include an α2 domain. In yet a further embodiment, the β1α1 molecules do not include either an α2 or a β2 domain.

Nucleic acid molecules encoding these domains may be produced by standard means, such as amplification by the polymerase chain reaction (PCR). Standard approaches for designing primers for amplifying open reading frames encoding these domains may be employed. Libraries suitable for the amplification of these domains include, for example, cDNA libraries prepared from the mammalian species in question; such libraries are available commercially, or may be prepared by standard methods. Thus, for example, constructs encoding the β1 and α1 polypeptides may be produced by PCR using four primers: primers B1 and B2 corresponding to the 5′ and 3′ ends of the β1 coding region, and primers A1 and A2 corresponding to the 5′ and 3′ ends of the α1 coding region. Following PCR amplification of the β1 and α1 domain coding regions, these amplified nucleic acid molecules may each be cloned into standard cloning vectors, or the molecules may be ligated together and then cloned into a suitable vector. To facilitate convenient cloning of the two coding regions, restriction endonuclease recognition sites may be designed into the PCR primers. For example, primers B2 and A1 may each include a suitable site such that the amplified fragments may be readily ligated together following amplification and digestion with the selected restriction enzyme. In addition, primers B1 and A2 may each include restriction sites to facilitate cloning into the polylinker site of the selected vector. Ligation of the two domain coding regions is performed such that the coding regions are operably linked, i.e., to maintain the open reading frame. Where the amplified coding regions are separately cloned, the fragments may be subsequently released from the cloning vector and gel purified, preparatory to ligation.

In certain embodiments, a peptide linker is provided between the β1 and α1 domains. Typically, this linker is between 2 and 25 amino acids in length, and serves to provide flexibility between the domains such that each domain is free to fold into its native conformation. The linker sequence may conveniently be provided by designing the PCR primers to encode the linker sequence. Thus, in the example described above, the linker sequence may be encoded by one of the B2 or A1 primers, or a combination of each of these primers.

3. Design of Recombinant MHC Class I α α1α2 Molecules

The amino acid sequences of mammalian MHC class I α chain proteins, as well as nucleic acids encoding these proteins, are well known in the art and available from numerous sources including GenBank. Exemplary sequences are provided in Browning et al. (1995) (human HLA-A); Kato et al. (1993) (human HLA-B); Steinle et al. (1992) (human HLA-C); Walter et al. (1995) (rat Ia); Walter et al. (1994) (rat Ib); Kress et al. (1983) (mouse H-2-K); Schepart et al. (1986) (mouse H-2-D); and Moore et al. (1982) (mouse H-2-1), which are incorporated by reference herein. In one embodiment, the MHC class I protein is a human MHC class I protein.

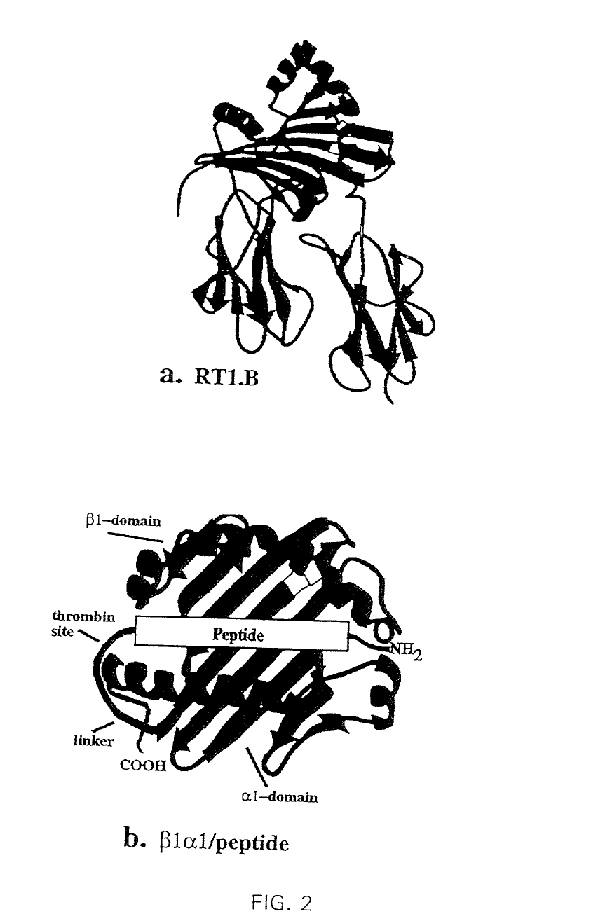

The recombinant MHC class I molecules of the present invention comprise the α1 domain of the MHC class I α chain covalently linked to the α2 domain of the MHC class I chain. These two domains are well defined in mammalian MHC class I proteins. Typically, the al domain is regarded as comprising about residues 1-90 of the mature chain and the α2 chain as comprising about amino acid residues 90-180, although again, the cut-off points are not precisely defined and will vary between different MHC class I molecules. The boundary between the α2 and α3 domains of the MHC class I α protein typically occurs in the region of amino acids 179-183 of the mature chain. The composition of the α1 and α2 domains may also vary outside of these parameters depending on the mammalian species and the particular α chain in question. One of skill in the art will appreciate that the precise numerical parameters of the amino acid sequence are much less important than the maintenance of domain function. An exemplary α1α2 molecule is depicted in FIG. 2. In one embodiment, the α1α2 molecule does not include an α3 domain.

The α1α2 construct may be most conveniently constructed by amplifying the reading frame encoding the dual-domain (α1 and α2) region between amino acid number 1 and amino acids 179-183, although one of skill in the art will appreciate that some variation in these end-points is possible. Such a molecule includes the native linker region between the α1 and α2 domains, but if desired that linker region may be removed and replaced with a synthetic linker peptide. The general considerations for amplifying and cloning the MHC class I α1 and α2 domains apply as discussed above in the context of the class II β1 and α1 domains.

4. Genetic Linkage of of Antigenic Polypeptide to β1α1 and α1α2 Molecules

The class β1α1 and class I α1α2 polypeptides of the invention are generally used in conjunction with an antigenic peptide. Any antigenic peptide that is conventionally associated with class I or class II MHC molecules and recognized by a T-cell can be used for this purpose. Antigenic peptides from a number of sources have been characterized in detail, including antigenic peptides from honey bee venom allergens, dust mite allergens, toxins produced by bacteria (such as tetanus toxin) and human tissue antigens involved in autoimmune diseases. Detailed discussions of such peptides are presented in U.S. Pat. Nos. 5,595,881, 5,468,481 and 5,284,935 incorporated herein by reference. Exemplary peptides include those identified in the pathogenesis of rheumatoid arthritis (type II collagen), myasthenia gravis (acetyl choline receptor), and multiple sclerosis (myelin basic protein).

As is well known in the art (see for example U.S. Pat. No. 5,468,481) the presentation of antigen in MHC complexes on the surface of APCs generally does not involve a whole antigenic peptide. Rather, a peptide located in the groove between the β1 and α1 domains (in the case of MHC II) or the α1 and α2 domains (in the case of MHC I) is typically a small fragment of the whole antigenic peptide. As discussed in Janeway & Travers (1997), peptides located in the peptide groove of MHC class I molecules are constrained by the size of the binding pocket and are typically 8-15 amino acids long, more typically 8-10 amino acids in length (but see Collins et al., 1994 for possible exceptions). In contrast, peptides located in the peptide groove of MHC class II molecules are not constrained in this way and are often much larger, typically at least 13 amino acids in length. Peptide fragments for loading into MHC molecules can be prepared by standard means, such as use of synthetic peptide synthesis machines.

The β1α1 and α1α2 molecules of the present invention may be “loaded” with peptide antigen in a number of ways, including by covalent attachment of the peptide to the MHC molecule. This may be conveniently achieved by operably linking a nucleic acid sequence encoding the selected peptide to the 5′ end of the construct encoding the MHC protein such that, in the expressed peptide, the antigenic peptide domain is linked to the N-terminus of β1 in the case of β1α1 molecules and cl in the case of α1α2 molecules. One convenient way of obtaining this result is to incorporate a sequence encoding the antigen into the PCR primers used to amplify the MHC coding regions. Typically, a sequence encoding a linker peptide sequence will be included between the molecules encoding the antigenic peptide and the MHC polypeptide. As discussed above, the purpose of such linker peptides is to provide flexibility and permit proper conformational folding of the peptides. For linking antigens to the MHC polypeptide, the linker should be sufficiently long to permit the antigen to fit into the peptide groove of the MHC polypeptide. Again, this linker may be conveniently incorporated into the PCR primers. However, as discussed in Example 1 below, it is not necessary that the antigenic peptide be ligated exactly at the 5′ end of the MHC coding region. For example, the antigenic coding region may be inserted within the first few (typically within the first 10) codons of the 5′ end of the MHC coding sequence.

This genetic system for linkage of the antigenic peptide to the MHC molecule is particularly useful where a number of MHC molecules with differing antigenic peptides are to be produced. The described system permits the construction of an expression vector in which a unique restriction site is included at the 5′ end of the MHC coding region (i.e., at the 5′ end of β1 in the case of β1α1-encoding constructs and at the 5′ end of α1 in the case of α1α2-encoding constructs). In conjunction with such a construct, a library of antigenic peptide-encoding sequences is made, with each antigen-coding region flanked by sites for the selected restriction enzyme. The inclusion of a particular antigen into the MHC molecule is then performed simply by (a) releasing the antigen-coding region with the selected restriction enzyme, (b) cleaving the MHC construct with the same restriction enzyme, and (c) ligating the antigen coding region into the MHC construct. In this manner, a large number of MHC-polypeptide constructs can be made and expressed in a short period of time.

An exemplary design of an expression cassette allowing simple exchange of antigenic peptides in the context of a β1α1 molecule is shown in FIG. 1. FIG. 1A shows the nucleic acid sequence encoding a prototype β1α1 molecule derived from rat MHC class II RT1.B, without the presence of the antigenic peptide. The position of the insertion site for the peptide and linker between the 5th and 6th (serine and proline) residues of the β1 domain is indicated by a ▾ symbol. In order to integrate the antigen coding region, a PCR primer comprising the sequence shown in FIG. 1B joined with additional bases from the FIG. 1A construct 3′ of the insertion site is employed in conjunction with a PCR primer reading from the 3′ end of the construct shown in FIG. 1A.) Amplification yields a product that includes the sequence shown in FIG. 1B integrated into the L IeI construct (i.e., with the antigenic peptide and linker sequences positioned between the codons encoding the 5th and 6th amino acid residues of the β1α1 sequence). In the case illustrated, the antigenic peptide is the MBP-72-89 antigen.

Notably, the MBP-72-89 coding sequence is flanked by unique Nco I and Spe I restriction enzyme sites. These enzymes can be used to release the MBP-72-89 coding region and replace it with coding regions for other antigens, for example those illustrated in FIGS. 1C and 1D.

The structure of the expressed β1α1 polypeptide with covalently attached antigen is illustrated in FIG. 2B; FIG. 2A shows the secondary structure of the complete RT1B molecule (including β1, β2, α1 and α2 domains).

Nucleic acid expression vectors including expression cassettes designed as explained above will be particularly useful for research purposes. Such vectors will typically include sequences encoding the dual domain MHC polypeptide (β1α1 or α1α2) with a unique restriction site provided towards the 5′ terminus of the MHC coding region, such that a sequence encoding an antigenic polypeptide may be conveniently attached. Such vectors will also typically include a promoter operably linked to the 5′ terminus of the MHC coding region to provide for high level expression of the sequences.

β1α1 and α1α2 molecules may also be expressed and purified without an attached peptide (as described in section 5 below), in which case they may be referred to as “empty”. The empty MHC molecules may then be loaded with the selected peptide as described in section 6 below.

5. Expression and Purification of Recombinant β1α1 and α1α2 Molecules In their most basic form, nucleic acids encoding the MHC polypeptides of the invention comprise first and second regions, having a structure A-B wherein, for class I molecules, region A encodes the class I α1 domain and region B encodes the class I α2 domain. For class II molecules, A encodes the class II α1 domain and B encodes the class II β1 domain. Where a linker sequence is included, the nucleic acid may be represented as B-L2-A, wherein L2 is a nucleic acid sequence encoding the linker peptide. Where an antigenic peptide is covalently linked to the MHC polypeptide, the nucleic acid molecule encoding this complex may be represented as P-B-A. A second linker sequence may be provided between the antigenic protein and the region B polypeptide, such that the coding sequence is represented as P-L2-B-L1-A. In all instances, the various nucleic acid sequences that comprise the MHC polypeptide (i.e., L1, L2, B, A and P) are operably linked such that the elements are situated in a single reading frame.

Nucleic acid constructs expressing these MHC polypeptides may also include regulatory elements such as promoters (Pr), enhancers and 3′ regulatory regions, the selection of which will be determined based upon the type of cell in which the protein is to be expressed. When a promoter sequence is operably linked to the open reading frame, the sequence may be represented as Pr-B-A, or (if an antigen-coding region is included) Pr-P-B-A, wherein Pr represents the promoter sequence. The promoter sequence is operably linked to the P or B components of these sequences, and the B-A or P-B-A sequences comprise a single open reading frame. The constructs are introduced into a vector suitable for expressing the MHC polypeptide in the selected cell type.

Numerous prokaryotic and eukaryotic systems are known for the expression and purification of polypeptides. For example, heterologous polypeptides can be produced in prokaryotic cells by placing a strong, regulated promoter and an efficient ribosome binding site upstream of the polypeptide-encoding construct. Suitable promoter sequences include the beta-lactamase, tryptophan (trp), 'phage T7 and lambda P_{L} promoters. Methods and plasmid vectors for producing heterologous proteins in bacteria are described in Sambrook et al. (1989). Suitable prokaryotic cells for expression of large amounts of ₂m fusion proteins include Escherichia coli and Bacillus subtilis. Often, proteins expressed at high levels are found in insoluble inclusion bodies; methods for extracting proteins from these aggregates are described by Sambrook et al. (1989, see ch. 17). Recombinant expression of MHC polypeptides in prokaryotic cells may alternatively be conveniently obtained using commercial systems designed for optimal expression and purification of fusion proteins. Such fusion proteins typically include a protein tag that facilitates purification. Examples of such systems include: the pMAL protein fusion and purification system (New England Biolabs, Inc., Beverly, Mass.); the GST gene fusion system (Amersham Pharmacia Biotech, Inc., Piscataway, N.J.); and the pTrcHis expression vector system (Invitrogen, Carlsbad, Calif.). For example, the pMAL expression system utilizes a vector that adds a maltose binding protein to the expressed protein. The fusion protein is expressed in E. coli. and the fusion protein is purified from a crude cell extract using an amylose column. If necessary, the maltose binding protein domain can be cleaved from the fusion protein by treatment with a suitable protease, such as Factor Xa. The maltose binding fragment can then be removed from the preparation by passage over a second amylose column.

The MHC polypeptides can also be expressed in eukaryotic expression systems, including Pichia pastoris, Drosophila, Baculovirus and Sindbis expression systems produced by Invitrogen (Carlsbad, Calif.). Eukaryotic cells such as Chinese Hamster ovary (CHO), monkey kidney (COS), HeLa, Spodoptera frugiperda, and Saccharomyces cerevisiae may also be used to express the MHC polypeptides. Regulatory regions suitable for use in these cells include, for mammalian cells, viral promoters such as those from CMV, adenovirus and SV40, and for yeast cells, the promoter for 3-phosphoglycerate kinase and alcohol dehydrogenase.

The transfer of DNA into eukaryotic, in particular human or other mammalian cells, is now a conventional technique. The vectors are introduced into the recipient cells as pure DNA (transfection) by, for example, precipitation with calcium phosphate or strontium phosphate, electroporation, lipofection, DEAE dextran, microinjection, protoplast fusion, or microprojectile guns. Alternatively, the nucleic acid molecules can be introduced by infection with virus vectors. Systems are developed that use, for example, retroviruses, adenoviruses, or Herpes virus.

An MHC polypeptide produced in mammalian cells may be extracted following release of the protein into the supernatant and may be purified using an immunoaffinity column prepared using anti-MHC antibodies. Alternatively, the MHC polypeptide may be expressed as a chimeric protein with, for example, b-globin. Antibody to b-globin is thereafter used to purify the chimeric protein. Corresponding protease cleavage sites engineered between the b-globin gene and the nucleic acid sequence encoding the MHC polypeptide are then used to separate the two polypeptide fragments from one another after translation. One useful expression vector for generating b-globin chimeric proteins is pSG5 (Stratagene, La Jolla, Calif.).

Expression of the MHC polypeptides in prokaryotic cells will result in polypeptides that are not glycosylated. Glycosylation of the polypeptides at naturally occurring glycosylation target sites may be achieved by expression of the polypeptides in suitable eukaryotic expression systems, such as mammalian cells.

Purification of the expressed protein is generally performed in a basic solution (typically around pH 10) containing 6M urea. Folding of the purified protein is then achieved by dialysis against a buffered solution at neutral pH (typically phosphate buffered saline (PBS) at around pH 7.4).

6. Antigen Loading of Empty β1α1 and α1α2 Molecules

Where the β1α1 and α1α2 molecules are expressed and purified in an empty form (i.e., without attached antigenic peptide), the antigenic peptide may be loaded into the molecules using standard methods. Methods for loading of antigenic peptides into MHC molecules is described in, for example, U.S. Pat. No. 5,468,481 herein incorporated by reference. Such methods include simple co-incubation of the purified MHC molecule with a purified preparation of the antigen.

By way of example, empty β1α1 molecules (1 mg/ml; 40 uM) may be loaded by incubation with a 10-fold molar excess of peptide (1 mg/ml; 400 uM) at room temperature, for 24 hours. Thereafter, excess unbound peptide may be removed by dialysis against PBS at 4 C for 24 hours. As is known in the art, peptide binding to β1α1 can be quantified by silica gel thin layer chromatography (TLC) using radiolabeled peptide. Based on such quantification, the loading may be altered (e.g., by changing the molar excess of peptide or the time of incubation) to obtain the desired result.

7. Other Considerations

a. Sequence Variants

While the foregoing discussion uses as examples naturally occurring MHC class I and class II molecules and the various domains of these molecules, one of skill in the art will appreciate that variants of these molecules and domains may be made and utilized in the same manner as described. Thus, reference herein to a domain of an MHC polypeptide or molecule (e.g., an MHC class II PI domain) includes both naturally occurring forms of the referenced molecule, as well as molecules that are based on the amino acid sequence of the naturally occurring form, but which include one or more amino acid sequence variations. Such variant polypeptides may also be defined in the degree of amino acid sequence identity that they share with the naturally occurring molecule. Typically, MHC domain variants will share at least 80% sequence identity with the sequence of the naturally occurring MHC domain. More highly conserved variants will share at least 90% or at least 95% sequence identity with the naturally occurring sequence. Variants of MHC domain polypeptides also retain the biological activity of the naturally occurring polypeptide. For the purposes of this invention, that activity is conveniently assessed by incorporating the variant domain in the appropriate β1α1 or α1α2 polypeptide and determining the ability of the resulting polypeptide to inhibit antigen specific T-cell proliferation in vitro, as described in detail below.

Variant MHC domain polypeptides include proteins that differ in amino acid sequence from the naturally occurring MHC polypeptide sequence but which retain the specified biological activity. Such proteins may be produced by manipulating the nucleotide sequence of the molecule encoding the domain, for example by site-directed mutagenesis or the polymerase chain reaction. The simplest modifications involve the substitution of one or more amino acids for amino acids having similar biochemical properties. These so-called conservative substitutions are likely to have minimal impact on the activity of the resultant protein. Table 1 shows examples of amino acids which may be substituted for an original amino acid in a protein and which are regarded as conservative substitutions.

TABLE 1

| Original Residue | Conservative Substitutions |
|---|---|
| Ala | ser |
| Asn | gln; his |
| Asp | glu |
| Cys | ser |
| Gln | asn |
| Glu | asp |
| Gly | pro |
| His | asn; gln |
| Ile | leu; val |
| Leu | ile; val |
| Lys | arg; gln |
| Met | leu; ile |
| Phe | met; leu; tyr |
| Ser | thr |
| Thr | ser |
| Trp | tyr |
| Tyr | trp; phe |
| Val | ile; leu |

More substantial changes in biological function or other features may be obtained by selecting substitutions that are less conservative than those shown above, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine. The effects of these amino acid substitutions or deletions or additions may be assessed through the use of the described T-cell proliferation assay.

At the nucleic acid level, one of skill in the art will appreciate that the naturally occurring nucleic acid sequences that encode class I and II MHC domains may be employed in the expression vectors, but that the invention is not limited to such sequences. Any sequence that encodes a functional MHC domain may be employed, and the nucleic acid sequence may be adapted to conform with the codon usage bias of the organism in which the sequence is to be expressed.

b. Incorporation of Detectable Markers

For certain in vivo and in vitro applications, the MHC molecules of the present invention may be conjugated with a detectable label. A wide range of detectable labels are known, including radionuclides (e.g., gamma-emitting sources such as indium-111), paramagnetic isotopes, fluorescent markers (e.g., fluorescein), enzymes (such as alkaline phosphatase), cofactors, chemiluminescent compounds and bioluminescent compounds. The binding of such labels to the MHC polypeptides may be achieved using standard methods. U.S. Pat. No. 5,734,023 (incorporated herein by reference) contains an extensive discussion of the labeling of MHC polypeptide derivatives using such labels. Where the detectable marker is to be covalently linked to the MHC molecule in a directed manner (i.e., rather than being randomly attached) it will generally be linked to the C terminus of the molecule so as to minimize interference with a peptide antigen linked at the N terminus.

c. Conjugation of Toxic Moieties

For certain uses of the disclosed MHC polypeptides, particularly in vivo therapeutic applications aimed at depleting certain T-cell populations, the polypeptides may be conjugated with a toxic moiety. Numerous toxic moieties suitable for disrupting T-cell function are known, including, but not limited to, protein toxins, chemotherapeutic agents, antibodies to a cytotoxic T-cell surface molecule, lipases, and radioisotopes emitting “hard” e.g., beta radiation. Examples of such toxins and methods of conjugating toxins to MHC molecules are described in U.S. Pat. No. 5,284,935 (incorporated herein by reference). Protein toxins include ricin, diphtheria and, Pseudomonas toxin. Chemotherapeutic agents include doxorubicin, daunorubicin, methotrexate, cytotoxin, and antisense RNA. Radioisotopes such as yttrium-90, phosphorus-32, lead-212, iodine-131, or palladium-109 may also be used. Where the toxic moiety is to be covalently linked to the MHC molecule in a directed manner (i.e., rather than being randomly attached) it will generally be linked to the C terminus of the molecule so as to minimize interference with a peptide antigen linked at the N terminus.

d. Pharmaceutical Formulations

For administration to animals, purified MHC polypeptides of the present invention are generally combined with a pharmaceutically acceptable carrier. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As is known in the art, protein-based pharmaceuticals may be only inefficiently delivered through ingestion. However, pill-based forms of pharmaceutical proteins may alternatively be administered subcutaneously, particularly if formulated in a slow-release composition. Slow-release formulations may be produced by combining the target protein with a biocompatible matrix, such as cholesterol. Another possible method of administering protein pharmaceuticals is through the use of mini osmotic pumps. As stated above a biocompatible carrier would also be used in conjunction with this method of delivery. Additional possible methods of delivery include deep lung delivery by inhalation (Edwards et al., 1997; Service, 1997) and trans-dermal delivery (Mitragotri et al., 1996).

It is also contemplated that the MHC polypeptides of the present invention could be delivered to cells in the nucleic acid form and subsequently translated by the host cell. This could be done, for example through the use viral vectors or liposomes. Liposomes could also be used for direct delivery of the polypeptides.

The pharmaceutical compositions of the present invention may be administered by any means that achieve their intended purpose. Amounts and regimens for the administration of the selected MHC polypeptides will be determined by the attending clinician. Effective doses for therapeutic application will vary depending on the nature and severity of the condition to be treated, the particular MHC polypeptide selected, the age and condition of the patient and other clinical factors. Typically, the dose range will be from about 0.1 ug/kg body weight to about 100 mg/kg body weight. Other suitable ranges include doses of from about 100 ug/kg to 1 mg/kg body weight. The dosing schedule may vary from once a week to daily depending on a number of clinical factors, such as the subject's sensitivity to the protein. Examples of dosing schedules are 3 ug/kg administered twice a week, three times a week or daily; a dose of 7 ug/kg twice a week, three times a week or daily; a dose of 10 ug/kg twice a week, three times a week or daily; or a dose of 30 ug/kg twice a week, three times a week or daily.

8. Exemplary Applications of Recombinant β1α1 and α1α2 Molecules

The class II β1α1 and class α1α2 polypeptides of the present invention are useful for a wide range of in vitro and in vivo applications. Indeed, as a result of the biological activities of these polypeptides, they may be used in numerous applications in place of either intact purified MHC molecules, or antigen presenting cells that express MHC molecules.

In vitro applications of the disclosed polypeptides include the detection, quantification and purification of antigen-specific T-cells. Methods for using various forms of MHC-derived complexes for these purposes are well known and are described in, for example, U.S. Pat. Nos. 5,635,363 and 5,595,881. For such applications, the disclosed polypeptides may be free in solution or may be attached to a solid support such as the surface of a plastic dish, a microtiter plate, a membrane, or beads. Typically, such surfaces are plastic, nylon or nitrocellulose. Polypeptides in free solution are useful for applications such as fluorescence activated sell sorting (FACS). For detection and quantification of antigen-specific T-cells, the polypeptides are preferably labeled with a detectable marker, such as a fluorescent marker.

The T-cells to be detected, quantified or otherwise manipulated are generally present in a biological sample removed from a patient. The biological sample is typically blood or lymph, but may also be tissue samples such as lymph nodes, tumors, joints etc. It will be appreciated that the precise details of the method used to manipulate the T-cells in the sample will depend on the type of manipulation to be performed and the physical form of both the biological sample and the MHC molecules. However, in general terms, the β1α1/peptide complex or α1α2/peptide complex is added to the biological sample, and the mixture is incubated for sufficient time (e.g., from about 5 minutes up to several hours) to allow binding. Detection and quantification of T-cells bound to the MHC/peptide complex may be performed by a number of methods including, where the MHC/peptide includes a fluorescent label, fluorescence microscopy and FACS. Standard immunoassays such as ELISA and RIA may also be used to quantify T-cell—MHC/peptide complexes where the MHC/peptide complexes are bound to a solid support. Quantification of antigen-specific T-cell populations will be especially useful in monitoring the course of a disease. For example, in a multiple sclerosis patient, the efficacy of a therapy administered to reduce the number of MBP-reactive T-cells may be monitored using MHC/MBP antigen complexes to quantify the number of such T-cells present in the patient. Similarly, the number of anti-tumor T-cells in a cancer patient may be quantified and tracked over the course of a therapy using MHC/tumor antigen complexes.

FACS may also be used to separate T-cell—MHC/peptide complexes from the biological sample, which may be particularly useful where a specified population of antigen-specific T-cells is to be removed from the sample, such as for enrichment purposes. Where the MHC/peptide complex is bound to magnetic beads, the binding T-cell population may be purified as described by Miltenyi et al (1990). By way of example, anti-tumor T-cells in the blood of a cancer patient may be purified using these methods, expanded in vitro and returned to the patient as part of an adoptive immunotherapy treatment.

A specified antigen-specific T-cell population in the biological sample may be anergized by incubation of the sample with MHC/peptide complexes containing the peptide recognized by the targeted T-cells. Thus, when these complexes bind to the TCR in the absence of other co-stimulatory molecules, a state of anergy is induced in the T-cell. Such an approach is useful in situations where the targeted T-cell population recognizes a self-antigen, such as in various autoimmune diseases. Alternatively, the targeted T-cell population may be killed directly by incubation of the biological sample with an MHC/peptide complex conjugated with a toxic moiety.

T-cells may also be activated in an antigen-specific manner by the polypeptides of the invention. For example, the disclosed MHC polypeptides loaded with a specified antigen may be adhered at a high density to a solid surface, such as a plastic dish or a magnetic bead. Exposure of T-cells to the polypeptides on the solid surface can stimulate and activate T-cells in an antigen-specific manner, despite the absence of co-stimulatory molecules. This is likely attributable to sufficient numbers of TCRs on a T-cell binding to the MHC/peptide complexes that co-stimulation is unnecessary for activation.

In one embodiment, suppressor T cells are induced. Thus, when the complexes bind to the TCR in the proper context, suppressor T cells are induced in vitro. In one embodiment, effector functions are modified, and cytokine profiles are altered by incubation with a MHC/peptide complex.

In vivo applications of the disclosed polypeptides include the amelioration of conditions mediated by antigen-specific T-cells. Such conditions include allergies, transplant rejection and autoimmune diseases including multiple sclerosis, rheumatoid arthritis, systemic lupus erythematosus, and insulin-dependent diabetes mellitus. Other researchers have described various forms of MHC polypeptides that may be used to treat these conditions and the methods used in those systems are equally useful with the MHC polypeptides of the present invention. Exemplary methodologies are described in U.S. Pat. Nos. 5,130,297, 5,284,935, 5,468,481, 5,734,023 and 5,194,425 (herein incorporated by reference). By way of example, the MHC/peptide complexes may be administered to a subject in order to induce anergy in self-reactive T-cell populations, or these T-cell populations may be treated by administration of MHC/peptide complexes conjugated with a toxic moiety. Alternatively, the MHC/peptide complexes may be administered to a subject to induce T suppressor cells or to modify a cytokine expression profile. The disclosed molecules may also be used to boost immune response in certain conditions such as cancer and infectious diseases.

### EXAMPLES

The following Examples illustrate certain aspects of the invention, but are not intended to limit in any manner, shape, or form, either explicitly or implicitly. While they are typical of those that might be used, other procedures, methodologies, or skill in the art may be used.

### Example 1

### Cloning, Expression and in vitro Folding of β1α1 Molecules

A prototypical nucleic acid construct was produced that encoded a single polypeptide chain with the amino terminus of the MHC class II α1 domain genetically linked to the carboxyl terminus of the MHC class II β1 domain. The sequence of this prototypical construct, made from the rat RT1B—and β-chain cDNAs is shown in FIG. 1A (SEQ ID NO:1).

RT1B α1- and β1-domain encoding cDNAs were prepared by PCR amplification of cloned RT1B α- and β-chain cDNA coding sequences (α6, β118, respectively) obtained from Dr. Konrad Reske, Mainz, FRG (Syha et al., 1989; Syha-Jedelhauser et al., 1991). The primers used to generate β1 were:

5′-AATTCCTCGAGATGGCTCTGCAGACCCC-3′ (XhoI 5′ primer) (SEQ ID NO:9); 5′-TCTTGACCTCCAAGCCGCCGCAGGGAGGTG-3′ (3′ ligation primer) (SEQ ID NO:10).

The primers used to generate α1 were:

5′-CGGCGGCTTGGAGGTCAAGACGACATTGAGG-3′ (5′ ligation primer) (SEQ ID NO:1 I); 5′-GCCTCGGTACCTTAGTTGACAGCTTGGGTTGAATTTG-3′ (KpnI 3′ primer) (SEQ ID NO: 12). Additional primers used were:

5′-CAGGGACCATGGGCAGAGACTCCCCA-3′ (NcoI 5′ primer) (SEQ ID NO:13); and 5′-GCCTCCTCGAGTTAGTTGACAGCTTGGGTT-3′ (XhoI 3′ primer) (SEQ ID NO:14).

Step one involved production of cDNAs encoding the β1 and α1 domains. PCR was conducted with Taq polymerase (Promega, Madison, Wis.) through 28 cycles of denaturation at 94.5° C. for 20 seconds, annealing at 55 C for 1.5 minutes and extension at 72° C. for 1.5 minutes, using β118 as template and the XhoI 5′ primer and 3′ ligation primer as primers and α6 cDNA as template and the 5′ ligation primer and KpnI 3′ primer. PCR products were isolated by agarose gel electrophoresis and purified using Gene-Clean (Bio 101, Inc., La Jolla, Calif.).

In step two, these products were mixed together without additional primers and heat denaturated at 94.5° C. for 5 minutes followed by 2 cycles of denaturation at 94.5° C. for 1 minute, annealing at 60° C. for 2 minutes and extension at 72° C. for 5 minutes. In step three, the annealed, extended product was heat denaturated at 94.5° C. for 5 minutes and subjected to 26 cycles of denaturation at 94.5° C. for 20 seconds, annealing at 60° C. for 1 minute and extension at 72° C. for 1 minute, in the presence of the XhoI 5′ primer and KpnI 3′ primer. The final PCR product was isolated by agarose gel electrophoresis and Gene-Cleaned. This produced a 656 base pair cDNA encoding the β1 1 molecule. The cDNA encoding the β1α1 molecule was moved into cloning vector pCR2.1 (Invitrogen, Carlsbad, Calif.) using Invitrogen's TA Cloning® kit. The cDNA in pCR2.1 was used as template and PCR was conducted through 28 cycles of denaturation at 94.5° C. for 20 seconds, annealing at 55 C for 1.5 minutes and extension at 72° C. for 1.5 minutes, using the NcoI 5′ primer and XhoI 3′ primer. The PCR products were cleaved with the relevant restriction enzymes and directionally cloned into pET21d+ (Novagen, Madison, Wis.; Studier et al., 1990). The constructs were confirmed by DNA sequencing. The β1α1 molecule used in these studies differs from wild-type in that it contains a beta-1 domain Q12R amino acid substitution.

For insertion of the peptide/linker cartridge (shown in FIG. 1A), the following approach was used. For insertion of the peptide/linker cartridge (shown in FIG. 1A), the following approach was used. The 210 bp peptide/linker cartridge was amplified using the XhoI 5′ primer and a primer of sequence:

5′-GAAATCCCGCGGGGAGCCTCCACCTCCAGAGCCTCGGGGCACTAGTGAGCC TCCACCTCCGAAGTGCACCACTGGGTTCTCATCCTGAGTCCTCTGGCTCTTCTGT GGGGAGTCTCTGCCCTCAGTCC-3′ (3′ -MBP-72-89/linker ligation primer) (SEQ ID NO:15) and the original full-length B1318 cDNA as a template. A 559 bp cDNA with α5′ overhang for annealing to the peptide/linker cartridge cDNA was generated using a primer: 5′-GCTCCCCGCGGGATTTCGTGTACCAGTTCAA-3′ (5′ peptide/linker ligation primer) (SEQ IDNO:16); and the Kpn I 3′ primer and the 656 bp β1α1 cDNA as the amplification template. Annealing and extension of the two cDNAs resulted in the 750 bp full-length Blal/MBP-72-89 construct. Modifications at the 5′ and 3′ ends of the β1α1 and β1α1/MBP-72-89 cDNAs were made for subcloning into pET21d+ (Novagen, Madison, Wis.; Studier et al., 1990) using the NcoI 5′ primer and the XhoI 3′ primer. The primers used to generate the MBP-55-69/linker cartridge were

5 ′-TATTACCATGGGCAGAGACTCCTCCGGCAAGGATTCGCATCATGCGGCGCG GACGACCCACTACGGTGGAGGTGGAGGCTCACTAGTGCCCC-3′ (5′ MBP-55-69 primer) (SEQ IDNO: 17) and

5 ′-GGGGCACTAGTGAGCCTCCACCTCCACCGTAGTGGGTCGTCCGCGCCGCATG ATGCGAATCCTTGCCGGAGGAGTCTCTGCCCATGGTAATA-3′ (3′ MBP-55-69 primer) (SEQ IDNO: 18). These were gel purified, annealed and then cut with NcoI and XhoI for ligation into β1α1/MBP-72-89 digested with NcoI and XhoI, to produce a plasmid encoding the β1α1/MBP-55-69 covalent construct. The primers used to generate the Guinea pig MBP-72-89/linker cartridge were

5′-TATTACCATGGGCAGAGACTCCCCACAGAAGAGCCAGAGGTCTCAGGATGA GAACCCAGTGGTGCACTTCGGAGGTGGAGGCTCACTAGTGCCCC -3′ (5′ Gp-MBP-72-89 primer) (SEQ IDNO:28) and

5′GGGGCACTAGTGAGCCTCCACCTCCGAAGTGCACCACTGGGTTCTCATCCTG AGACCTCTGGCTCTTCTGTGGGGAGTCTCTGCCCATGGTAAT-3′ (3′ Gp-MBP-72-89 primer) (SEQ IDNO:29). These were gel purified, annealed and then cut with NcoI and XhoI for ligation into β1α1/MBP-72-89 digested with NcoI and XhoI, to produce a plasmid encoding the β1α1/Gp-MBP-72-89 covalent construct. The primers used to generate the CM-2/linker cartridge were

5′ -TATTACCATGGGCAGAGACTCCAAACTGGAACTGCAGTCCGCTCTGGAAGA AGCTGAAGCTTCCCTGGAACACGGAGGTGGAGGCTCACTAGTGCCCC-3′ (5′ CM-2 primer) (SEQ IDNO:19) and

5′ -GGGGCACTAGTGAGCCTCCACCTCCGTGTTCCAGGGAAGCTTCAGCTTCTTC CAGAGCGGACTGCAGTTCCAGTTTGGAGTCTCTGCCCATGGTAATA-3′ (3′CM-2 primer) (SEQ ID NO:20). These were gel purified, annealed and then cut with NcoI and XhoI for ligation into β1α1/MBP-72-89 digested with NcoI and XhoI, to produce a plasmid encoding the β1α1/CM-2 covalent construct.

Protein expression was tested in a number of different E. coli strains, including a thioredoxin reductase mutant which allows disulfide bond formation in the cytoplasm (Derman et al., 1993). With such a small molecule, it became apparent that the greatest yield of material could be readily obtained from inclusion bodies, refolding the protein after solubilization and purification in buffers containing 6M urea. Accordingly, E. coli strain BL21(DE3) cells were transformed with the pET21d+ construct containing the β1α1-encoding sequence. Bacteria were grown in one liter cultures to mid-logarithmic phase (OD₆₀₀=0.6-0.8) in Luria-Bertani (LB) broth containing carbenicillin (50 μg/ml) at 37° C. Recombinant protein production was induced by addition of 0.5 mM isopropyl β-D-thiogalactoside (IPTG). After incubation for 3 hours, the cells were centrifuged and stored at −80° C. before processing. All subsequent manipulations of the cells were at 4° C. The cell pellets were resuspended in ice-cold PBS, pH 7.4, and sonicated for 4×20 seconds with the cell suspension cooled in a salt/ice/water bath. The cell suspension was then centrifuged, the supernatant fraction was poured off, the cell pellet resuspended and washed three times in PBS and then resuspended in 20 mM ethanolarnine/6 M urea, pH 10, for four hours. After centrifugation, the supernatant containing the solubilized recombinant protein of interest was collected and stored at 4° C. until purification. Recombinant β1α1 construct was purified and concentrated by FPLC ion-exchange chromatography using Source 30Q anion-exchange media (Pharmacia Biotech, Piscataway, N.J.) in an XK26/20 column (Pharmacia Biotech), using a step gradient with 20 mM ethanolamine/6M urea/IM NaCl, pH 10. The homogeneous peak of the appropriate size was collected, dialyzed extensively against PBS at 4° C., pH 7.4, and concentrated by centrifugal ultrafiltration with Centricon-10 membranes (Amicon, Beverly, Mass.). The dialysis step, which removed the urea from the protein preparation and reduced the final pH, resulted in spontaneous re-folding of the expressed protein. For purification to homogeneity, a finish step used size exclusion chromatography on Superdex 75 media (Pharmacia Biotech) in an HR16/50 column (Pharmacia Biotech). The final yield of purified protein varied between 15 and 30 mg/L of bacterial culture.

Conformational integrity of the molecules was demonstrated by the presence of a di sulfide bond between cysteines β15 and β79 as detected on gel shift assay, and the authenticity of the purified protein was verified using the OX-6 monoclonal antibody specific for RT1B by Western Blotting (data not shown). Circular dichroism (CD) reveals that the β1α1 molecules have highly ordered secondary structures. The empty β1α1 molecule contains approximately 30% alpha-helix, 15% beta-strand, 26% beta-turn, and 29% random coil structures. Comparison with the secondary structures of class II molecules determined by x-ray crystallography provides strong evidence that the β1α1 molecules share the beta-sheet platform/anti-parallel alpha-helix secondary structure common to all class II antigen binding domains. Furthermore, thermal denaturation revealed a high degree of cooperativity and stability of the molecules (data not shown).

### Example 2

### β1α1Molecules Bind T Lymphocytes in an Epitope-Specific Manner

The β1α1 molecule produced as described above was tested for efficacy (T-cell binding specificity) using the Experimental Autoimmune Encephalomyelitis (EAE) system. EAE is a paralytic, inflammatory, and sometimes demyelinating disease mediated by CD4+ T cells specific for central nervous system myelin components including myelin basic protein (MBP). EAE shares similar immunological abnormalities with the human demyelinating disease MS (Paterson, 1981) and has been a useful model for testing preclinical therapies for the human illness (Weiner et al, 1993; Vandenbark et al., 1989; Howell et al., 1989; Oksenberg et al., 1993; Yednock et al, 1992; Jameson et al., 1994; Vandenbark et al., 1994). In Lewis rats, the dominant encephalitogenic MBP epitope resides in the 72-89 peptide (Bourdette et al., 1991). Onset of clinical signs of EAE occurs on day 10-11, and the disease lasts four to eight days. The majority of invading T lymphocytes are localized in the CNS during this period.

Materials and Methods

Test and control peptides for loading into the purified β1α1 molecules were synthesized as follows: Gp-MBP-69-89 peptide (GSLPQKSQRSQDENPVVHF) (SEQ ID NO:25), rat-MBP-69-89 peptide (GSLPQKSQRTQDENPVVHF) (SEQ ID NO:30), Gp-MBP-55-69 peptide (SGKDSHHAARTTHYG) (SEQ ID NO:26), and cardiac myosin peptide CM-2 (KLELQSALEEAEASLEH) (SEQ ID NO:27) (Wegmann et al., 1994) were prepared by solid-phase techniques (Hashim et al., 1986). The Gp-MBP peptides are numbered according to the bovine MBP sequence (Vandenbark et al., 1994; Martenson, 1984). Peptides were loaded onto MBP at a 1:10 protein:peptide molar ratio, by mixing at room temperature for 24 hours, after which all subsequent manipulations were performed at 4° C. Free peptide was then removed by dialysis or centrifugal ultrafiltration with Centricon-10 membranes, serially diluting and concentrating the solution until free peptide concentration was less than 2 μM.

T-cell lines and the A1 hybridoma were prepared as follows: Short-term T-lymphocyte lines were selected with MBP-69-89 peptide from lymph node cells of naive rats or from rats immunized 12 days earlier with Gp-MBP/CFA as described byvandenbark et al., 1985) The rat VB8.2+ T cell hybridoma C14/BW12-12A1 (A1) used in this study has been described previously (Burrows et al., 1996). Briefly, the Al hybridoma was created by fusing an encephalitogenic LEW(RT1¹) T cell clone specific for Gp-BP-72-89 (White et al., 1989; Gold et al, 1991) with a TCR (α/β) negative thymoma, BW5147 (Golding et al., 1985). Wells positive for cell growth were tested for IL-2 production after stimulation with antigen in the presence of APCs (irradiated Lewis rat thymocytes) and then subdloned at limiting dilution. The Al hybridoma secretes IL-2 when stimulated in the presence of APCs with whole Gp-BP or Gp-BP-69-89 peptide, which contains the minimum epitope, MBP-72-89.

Two color immunofluorescent analysis was performed on a FACScan instrument (Becton Dickinson, Mountain View, Calif.) using CellQues™ software. Quadrants were defined using non-relevant isotype matched control antibodies. MBP-molecules with and without loaded peptide were incubated with the A1 hybridoma (10 μM β1α1/peptide) for 17 hours, 4° C., washed three times, stained with fluorochrome (FITC or PE) conjugated antibodies specific for rat class II (OX6-PE), and TCR V138.2 (PharMingen, San Diego, Calif.) for 15 minutes at room temperature, and analyzed by flow cytometry. The CM-2 cell line was blocked for one hour with unconjugated OX6, washed and then treated as the Al hybridoma. Staining media was PBS, 2% fetal bovine serum, 0.01% azide.

Results

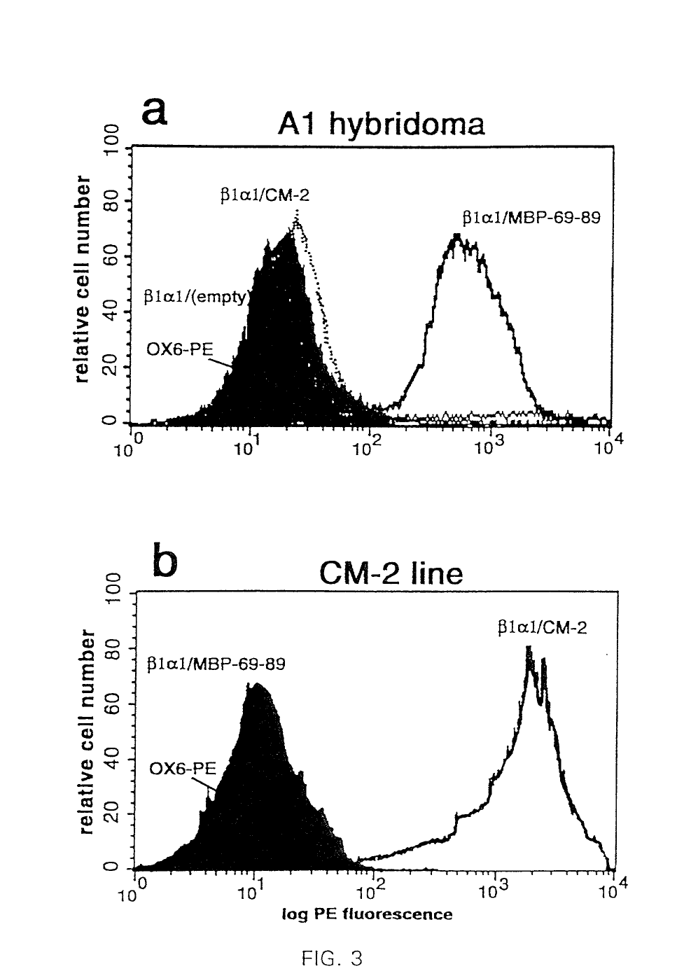

Epitope-specific binding was evaluated by loading the β1α1 molecule with various peptides and incubating β1α1/peptide complexes with the Al hybridoma that recognizes the MBP-72-89 peptide (Burrows et al., 1997), or with a cardiac myosin CM-2-specific cell line. As is shown in FIG. 3A, the β1α1 construct loaded with MBP-69-89 peptide (β1α1/MBP-69-89) specifically bound to the A1 hybridoma, with a mean fluorescence intensity (MFI) of 0.8×10³ Units, whereas the β1α1 construct loaded with CM-2 peptide (β1α1/CM-2) did not stain the hybridoma. Conversely, β1α1/CM-2 specifically bound to the CM-2 line, with a MFI of 1.8×10³ Units, whereas the Blol/MBP-69-89 complex did not stain the CM-2 line (FIG. 3B). The β1α1 construct without exogenously loaded peptide does not bind to either the Al hybridoma (FIG. 3A) nor the CM-2 line (data not shown). Thus, bound epitope directed the specific binding of the β1α1/peptide complex.

### Example 3

### β1α1 Molecules Conjugated with a Fluorescent Label

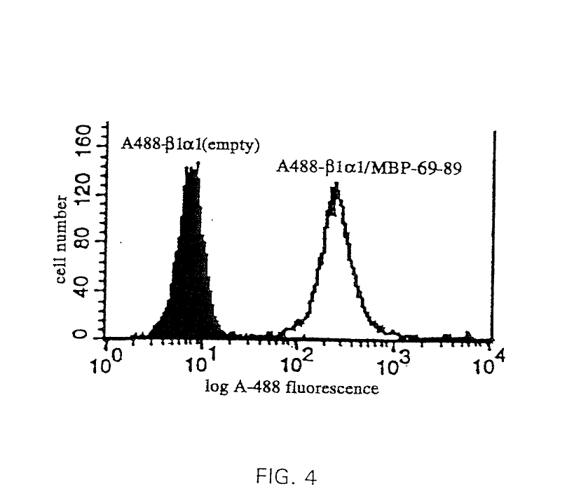

To avoid using a secondary antibody for visualizing the interaction of β1α1/peptide molecules with TCR (such as OX-6, used above), a 131I1 molecule directly conjugated with a chromophore was produced. The Alexa-488™ dye (A488; Molecular Probes, Eugene, Oreg.) has a spectra similar to fluorescein, but produces protein conjugates that are brighter and more photo-stable than fluorescein conjugates. As is shown in FIG. 4, A488-conjugated β1α1 (molar ratio dye/protein=1), when loaded with MBP-69-89, bound to the Al hybridomas (MCI=300 Units), whereas empty β1α1 did not.

### Example 4

### β1α1 Molecules Inhibit Epitope-Specific T-Cell Proliferation in vitro

T-cell proliferation assays were performed to evaluate the effect of the constructs on T cell activation.

Materials and Methods

Proliferation assays were performed in 96-well plates as described previously (Vandenbark et al., 1985). Briefly, 4×10⁵ cells in 200 μl/well (for organ stimulation assays) or 2×10⁴ T cells and 1×10⁶ irradiated APCs (for short-term T cell lines) were incubated in RPMI and 1% rat serum in triplicate wells with stimulation medium only, Con A, or antigen with or without supplemental IL-2 (20 Units/ml) at 37 C in 7% CO₂. The cultures were incubated for three days, the last 18 hr in the presence of [³H]thymidine (0.5 μCi/10 μl/well). The cells were harvested onto glass fiber filters and [³H]thymidine uptake assessed by liquid scintillation. In some experiments, the T cells were pretreated 24 hours with β1α1 constructs (with and without loaded peptides), washed, and then used in proliferation assays with and without IL-2, as above. Mean counts per minute ±SD were calculated from triplicate wells and differences between groups determined by Student's t-test.

Results

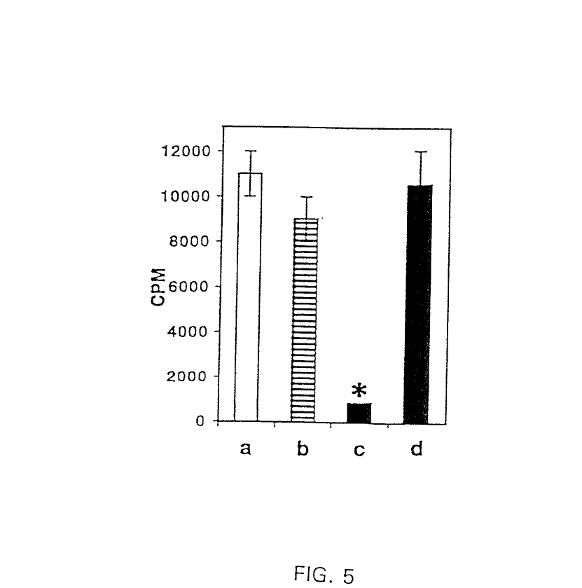

A range of concentrations (10 nM to 20 μM) of peptide-loaded β1α1 complexes were pre-incubated with an MBP-69-89 specific T cell line prior to stimulation with the MBP-69-89 peptide ±APC (antigen-presenting cell). As is shown in FIG. 5, pre-treatment of MBP-69-89 specific T cells with 10 nM β1α1/MBP-69-89 complex significantly inhibited proliferation (>90%), whereas pre-incubation with 20 μM β1α1/MBP-55-69 complex produced a nominal (27%) but insignificant inhibition. Of mechanistic importance, the response inhibited by the β1α1/MBP-69-89 complex could be fully restored by including 20 Units/ml of IL-2 during stimulation of the T cell line (FIG. 5) suggesting that the T-cells had been rendered anergic by exposure to the β1α1/MBP-69-89 complex.

### Example 5

### Antigen-Loaded β1α1 Molecules Suppress and Treat EAE

The β1α1/MBP-69-89 complex was evaluated for its ability to suppress the induction, as well as to treat existing signs of EAE in Lewis rats.

Materials and Methods

Female Lewis rats (Harlan Sprague-Dawley, Inc., Indianapolis, Ind.), 8-12 weeks of age, were used for clinical experiments in this study. The rats were housed under germ-free conditions at the Veterans Affairs Medical Center Animal Care Facility, Portland, Oreg., according to institutional guidelines. Active EAE was induced in the rats by subcutaneous injection of 25 μg guinea pig myelin basic protein (GP-MBP) or 200 jig GP-MBP-69-89 peptide in Freund's complete adjuvant supplemented with 100 or 400 jig Mycobacterium tuberculosis strain H37Ra (Difco, Detroit, Mich.), respectively. The clinical disease course induced by the two emulsions was essentially identical, with the same day of onset, duration, maximum severity, and cumulative disease index. The rats were assessed daily for changes in clinical signs according to the following clinical rating scale: 0, no signs; 1, limp tail; 2, hind leg weakness, ataxia; 3, paraplegia; and 4, paraplegia with forelimb weakness, moribund condition. A cumulative disease score was obtained by summing the daily disability scores over the course of EAE for each affected rat, and a mean cumulative disease index (CDI) was calculated for each experimental group.

Spinal cord mononuclear cells were isolated by a discontinuous percol gradient technique and counted as previously described (Bourdette et al., 1991). The cells were stained with fluorochrome (FITC or PE) conjugated antibodies specific for rat CD4, CD8, CD11b, CD45ra, TCR V138.2 and CD134 (PharMingen, San Diego, Calif.) for 15 min at room temperature and analyzed by flow cytometry. The number of positive staining cells per spinal cord was calculated by multiplying the percent staining by the total number of cells per spinal cord. Control and β1α1/MBP-69-89 protected rats were sacrificed at peak and recovery of clinical disease, spinal cords were dissected and fixed in 10% buffered formalin. The spinal cords were paraffin-embedded and sections were stained with luxol fast blue-periodic acid schiff-hematoxylin for light microscopy.

Results

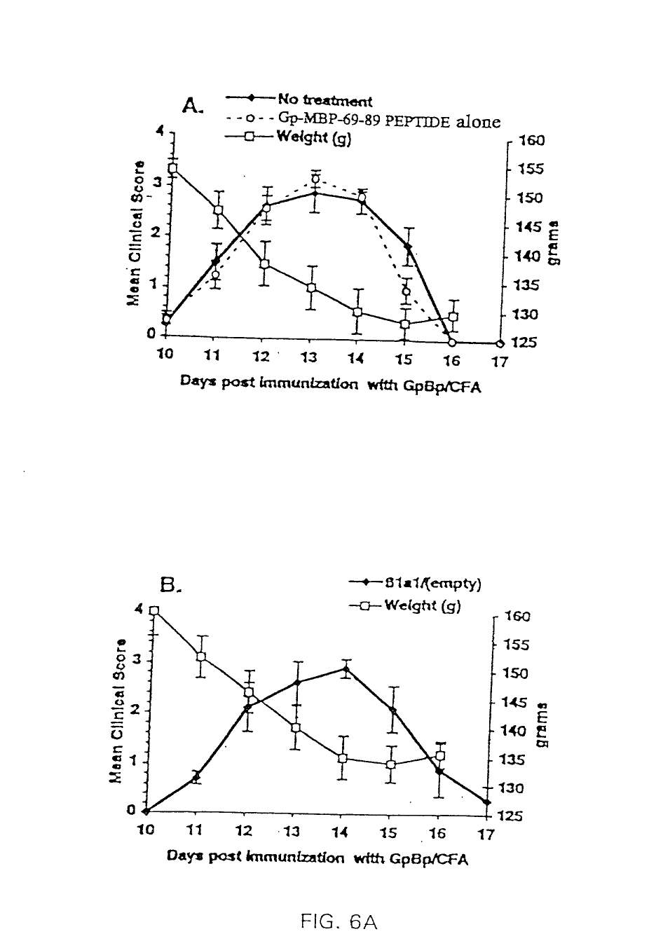

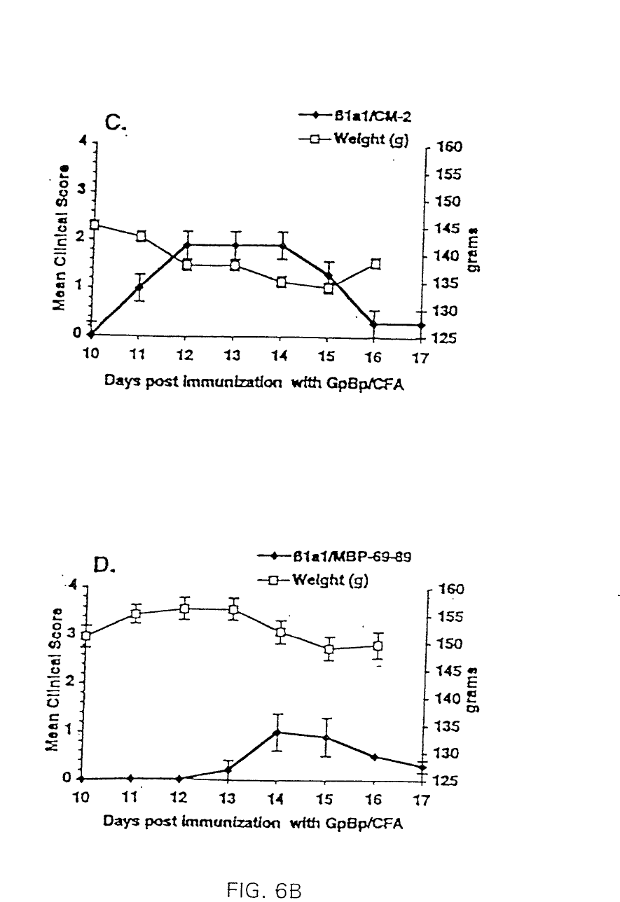

Intravenous injection (i.v.) of 300 μg of the β1α1/MBP-69-89 complex in saline on days 3, 7, 9, 11, and 14 after injection of MBP or MBP-69-89 peptide in CFA suppressed the induction of clinical (FIG. 6 and Table 3) and histological (not shown) signs of EAE. Injection of as little as 30 μg of the β1α1/MBP-69-89 complex following the same time course was also effective, completely suppressing EAE in 4 of 6 rats, with only mild signs in the other 2 animals. All of the control animals that were untreated, that received 2 μg MBP-69-89 peptide alone (the dose of free peptide contained in 30 μg of the complex), or that received 300 μg of the empty β1α1 construct developed a comparable degree of paralytic EAE (Table 2). Interestingly, injection of 300 μg of a control β1α1/CM-2 peptide complex produce a mild (about 30%) suppression of EAE (FIG. 6 and Table 2). In parallel with the course of disease, animals showed a dramatic loss in body weight (FIG. 6), whereas animals treated with the β1α1/MBP-69-89 complex showed no significant loss of body weight throughout the course of the experiment.

TABLE 2
Effect of β1α1/peptide complexes on EAE in Lewis rats.

| | | | | Maximum | |
|---|---|---|---|---|---|
| | | Day of | Duration | Disease | Cumulative |
| Treatment of EAE^{a} | Incidence | Onset | (days) | Score | Disease Index |
| Untreated^{b} | 11/11 | 12 ± 1^{c} | 5 ± 1 | 2.9 ± 0.3 | 10.0 ± 2.2 |
| 2 μg MBP-69-89 | 6/6 | 12 ± 1 | 6 ± 1 | 3.3 ± 0.3 | 11.2 ± 1.9 |
| β1α1/(empty) | 5/5 | 12 ± 1 | 6 ± 1 | 2.9 ± 0.6 | 9.7 ± 2.1 |
| 300 μg | | | | | |
| β1α1/CM-2 | 5/5 | 12 ± 1 | 6 ± 2 | 1.9 ± 0.8 | 7.2 ± 2.6* |
| 300 μg | | | | | |
| β1α1/MBP-69-89 | 0/6* | — | — | 0 ± 0** | 0 ± 0** |
| 300 μg | | | | | |
| β1α1/MBP-69-89 | 2/6 | 14 ± 0 | 4 ± 0 | 0.2 ± 0.1** | 0.7 ± 0.3** |
| 30 μg | | | | | |

^{a}EAE was induced with either Gp-BP/CFA or MBP-69-89/CFA.
^{b}Combined controls from two experiments.
^{c}Values represent the mean ± S.D.
*P 0.05
**P 0.01

TABLE 3
Characterization of infiltrating spinal cord cells at the peak of EAE in
control and β1α1/MBP-69-89 protected rats.

| Spinal cord | Total* | OX40+ | Vβ8.2+ | Vβ8.2+/OX40+ |
|---|---|---|---|---|
| Protected | 200 | 38 | 10 | 5 |
| Control | 7500 | 1750 | 980 | 667 |

*Number of cells/spinal cord × 10⁻³

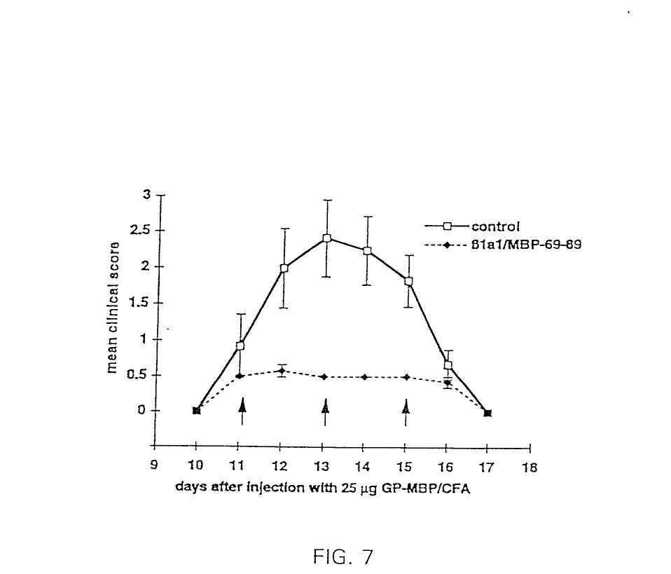

To evaluate the effect of the construct on established disease, Lewis rats were treated with 300 μg of the β1α1/MBP-69-89 complex on the first day of disease onset, with follow-up injections 48 and 96 hours later. EAE in the control rats progressed to complete hind limb paralysis, whereas no progression of the disease occurred in any of the treated animals (FIG. 7). The mild course of EAE (mean cumulative index, MCI=3±0.13) in the treated group was significantly less than the severe course of EAE in the control group (MCI=11.2±2.7, p=0.013), although the duration of disease (6 days) was the same in both groups.

Consistent with the complete lack of inflammatory lesions in spinal cord histological sections (not shown), suppression of EAE with the β1α1/MBP-69-89 complex essentially eliminated the infiltration of activated inflammatory cells into the CNS. Mononuclear cells were isolated from the spinal cords of control and protected animals at peak and recovery of clinical disease and examined by FACS analysis. The total number of mononuclear cells isolated from spinal cords of control animals at peak of clinical disease (day 14) was 40-fold higher than from protected animals evaluated at the same time point (Table 3). Moreover, protected animals had 72% fewer activated (OX40+), V138.2+ T cells in the spinal cord when compared to control animals (Table 3). CD4+ and CD8+ T cells, macrophages and B cell numbers were also significantly reduced in protected animals (not shown). The number of nmononuclear cells isolated after recovery from EAE was reduced 4.5-fold in protected animals (0.64×10⁵ cells/spinal cord) compared to control animals (2.9×10⁵ cells/spinal cord). Protected animals also had 10-fold fewer activated (OX40+), VB38.2+ T cells in the spinal cord than control animals after recovery from disease.

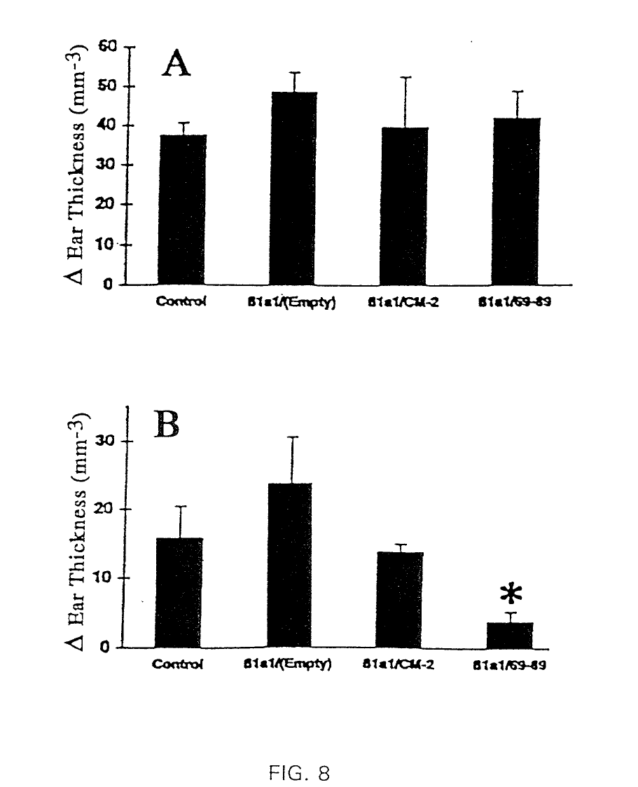

Treatment with β1α1/MBP-69-89 complex specifically inhibited the delayed-type hypersensitivity (DTH) response to MBP-69-89. As shown in FIG. 8A, changes in ear thickness 24 hours after challenge with PPD were uneffected by in animals treated with β1 1 or β1α1 loaded with peptides. However, as is shown in FIG. 8B, while animals treated with β1α1 alone or complexed with CM-2 had no effect on the DTH response, animals treated with the β1α1/MBP-69-89 complex showed a dramatic inhibition of the DTH response to MBP-69-89.

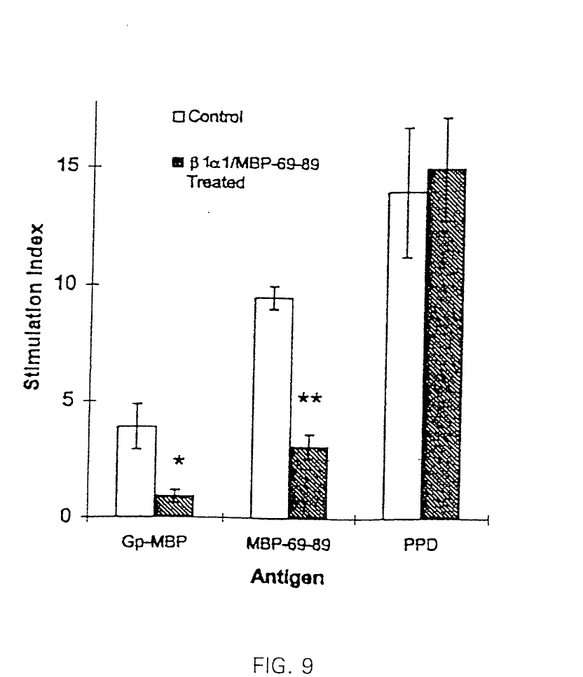

Treatment of EAE with the β1α1/MBP-69-89 complex also produced an inhibition of lymph node (LN) T cell responses. As is shown in FIG. 9, LN cells from rats treated with the suppression protocol (FIG. 6) were inhibited 2-4 fold in response to MBP or the MBP-69-89 peptide compared to control rats. This inhibition was antigen specific, since LN T cell responses to PPD (stimulated by the CFA injection) were the same in treated and control groups. T cell responses tested in rats treated after disease onset (FIG. 7) were also inhibited, in an IL-2 reversible manner. LN cell responses to MBP and MBP-69-89 peptide were optimal (S.I=4-5×) at low antigen (Ag) concentrations (4 μg/ml), and could be enhanced 2-fold with additional IL-2. In contrast, responses were inhibited in treated rats, with optimal LN cell responses (±3×) requiring higher Ag concentrations (20-50 μg/ml). However, in the presence of IL-2, responses could be restored to a level comparable to control rats (S.I.=6-11×) without boosting Ag concentrations.

In the presented Examples, polypeptides comprising the MHC class II Bi and al domains are described. These molecules lack the β2 domain, the β2 domain known to bind to CD4, and transmembrane and intra-cytoplasmic sequences. The reduced size and complexity of the β1α1 construct permits expression and purification of the molecules from bacterial inclusion bodies in high yield. The β1α1 molecules are shown to refold in a manner that allows binding of allele-specific peptide epitopes and to have excellent solubility in aqueous buffers. When complexed with peptide antigen, direct detection of the β1α1/peptide complexes to T cells can be visualized by FACS, with the specificity of binding determined by the peptide antigen. The β1α1/69-89 complex exerted powerful and selective inhibitory effects on T cell activation in vitro and in vivo. Because of its simplicity, biochemical stability, biological properties, and structural similarity with human class II homologs, the β1α1 construct represents a template for producing a novel class of TCR ligands.

Direct binding studies using the Al hybridoma specific for MBP-72-89 showed distinct staining with β1α1/MBP-69-89, with a 10-fold increase in MFI over background, and was not stained with β1α1/CM-2 nor “empty” β1α1. In a reciprocal manner, binding studies using a CM-2 specific cell line showed strong staining with β1α1/CM-2 and no staining with β1α1/MBP-69-89. Thus, bound epitope directed specific interaction of the β1α1/peptide complexes. Identification of antigen-specific T cells has been possible in a few systems (McHeyzer et al., 1995; MacDonald et al., 1993; Walker et al., 1995; Reiner et al., 1993), using labeled anti-idiotypic T cell receptor antibodies as specific markers, but the general approach of staining specific T cells with their ligand has failed because soluble peptide-MHC complexes have an inherently fast dissociation rate from the T cell antigen receptor (Corr et al., 1995; Matsui et al., 1994; Syulkev et al., 1994). Multimeric peptide-MHC complexes containing four-domain soluble MHC molecules have been used to stain antigen-specific T lymphocytes (Altman et al., 1996), with the ability to bind more than one T cell receptor (TCR) on a single T cell presumably giving the multimeric molecules a correspondingly slower dissociation rate. Staining with Blal/peptide complexes, while specific, did take an incubation period of approximately 10 hours to saturate (data not shown). The extraordinarily bright staining pattern of the Al hybridoma with the β1α1/MBP-69-89 complex, and the CM-2 line with β1α1/CM-2, coupled with the length of time it takes to achieve binding saturation, suggests that this molecule might have a very slow off-rate once bound to the TCR. These complexes and modified versions of them would be unusually well suited to directly label antigen-specific T cells for purposes of quantification and recovery.

The β1α1/peptide complex was highly specific in its ability to bind to and inhibit the function of T cells. In vitro proliferation of MBP-specific T cells was inhibited >90% with the β1α1/MBP-69-89 complex, and in vivo there was a nearly complete inhibition of clinical and histological EAE.

The most profound biological activity demonstrated for β1α1/MBP-69-89 was its ability to almost totally ablate the encephalitogenic capacity of MBP-69-89 specific T cells in vivo. Injection of this complex after initiation of EAE nearly completely suppressed clinical and histological signs of EAE, apparently by directly inhibiting the systemic activation of MBP-69-89 specific T cells, and preventing recruitment of inflammatory cells into the CNS. Moreover, injection of β1α1/MBP-69-89 after onset of clinical signs arrested disease progression, demonstrating the therapeutic potential of this molecular construct. Interestingly, the effect of the complex on already activated T cells was not only to inhibit stimulation, but also to reduce sensitivity to antigen, with optimal activation after treatment requiring a 10-fold increase in antigen concentration.

From a drug engineering and design perspective this prototypic molecule represents a major breakthrough. The demonstrated biological efficacy of the β1α1/MBP-69-89 complex in EAE raises the possibility of using this construct as a template for engineering human homologs for treatment of autoimmune diseases such as multiple sclerosis, that likely involves inflammatory T cells directed at CNS proteins. One candidate molecule would be HLA-DR2/MBP-84-102, which includes both the disease-associated class II allele and a known immunodominant epitope that has been reported to be recognized more frequently in MS patients than controls. However, because of the complexity of T cell response to multiple CNS proteins and their component epitopes, it is likely that a more general therapy may require a mixture of several MHC/Ag complexes. The precision of inhibition induced by the novel β1α1/MBP-69-89 complex reported herein represents an important first step in the development of potent and selective human therapeutic reagents. With this new class of reagent, it may be possible to directly quantify the frequency and prevalence of T cells specific for suspected target autoantigens, and then to selectively eliminate them in affected patients. Through this process of detection and therapy, it may then be possible for the first time to firmly establish the pathogenic contribution of each suspected T cell specificity.

### Example 6

### Design, Engineering and Production of Human Recombinant T Cell Receptor Ligands Derived from HLA-DR2 Experimental Procedures

Homology Modeling

Sequence alignment of MHC class II molecules from human, rat and mouse species provided a starting point for these studies (Burrows et al., 1999). Graphic images were generated with the program Sybyl (Tripos Associates, St. Louis, Mo.) and an O2 workstation (IRIX 6.5, Silicon Graphics, Mountain View, Calif.) using coordinates deposited in the Brookhaven Protein Data Bank (Brookhaven National Laboratories, Upton, N.Y.). Structure-based homology modeling was based on the refined crystallographic coordinates of human DR2 (Smith et al., 1998; Li et al., 2000) as well as DRI (Brown et al., 1996; Murthy et al., 1997), murine I-E^{k} molecules (Fremont et al., 1996), and scorpion toxins (Zhao et al., 1992; Housset et al., 1994; Zinn-Justin et al., 1996). Amino acid residues in human DR2 (PDB accession numbers 1BX2) were used. Because a number of residues were missing/not located in the crystallographic data (Smith et al., 1998), the correct side chains were inserted and the peptide backbone was modeled as a rigid body during structural refinement using local energy minimization.

Recombinant TCR Ligands (RTLs)

For production of the human RTLs, mRNA was isolated (Oligotex Direct mRNA Mini Kit; Qiagen, Inc., Valencia, Calif.) from L466.1 cells grown in RPMI media. First strand cDNA synthesis was carried out using SuperScript II Rnase H-reverse transcriptase (Gibco BRL, Grand Island, N.Y.).

Using the first strand reaction as template source, the desired regions of the DRB*1501 and DRA*0101 DNA sequences were amplified by PCR using Taq DNA polymerase (Gibco BRL, Grand Island, N.Y.), with an annealing temperature of 55° C. The primers used to generate β1 were 5′-ATTACCATGGGGGACACCCGACCACGTTT-3′ (huNcoI→, SEQ ID NO:28) and

5′ -GGATGATCACATGTTCTTCTTTGATGACTCGCCGCTGCACTGTGA-3′ (hu β1α1 Lig←, SEQ ID NO:29). The primers used to generate α1 were

5′ -TCACAGTGCAGCGGCGAGTCATCAAAGAAGAACATGTGATCATCC-3′ (hu β1α1 Lig←, SEQ ID NO:30) and

5′ -TGGTGCTCGAGTTAATTGGTGATCGGAGTATAGTTGG-3′ (huXhoI←, SEQ ID NO:31).

The amplification reactions were gel purified, and the desired bands isolated (QIAquick Gel Extraction Kit; Qiagen, Inc., Valencia, Calif.). The overhanging tails at the 5′-end of each primer added overlapping segments and restriction sites (NcoI and XhoI) at the ends of each PCR amplification product. The two chains were linked in a two step PCR reaction. In the first step, 5 μl of each purified amplification product were added to a 50 μl primer free PCR reaction, and cycled five times at an annealing temperature of 55° C. A 50 μl reaction mix containing the huNcoI X→ and huXhoI←primers was then added directly to the initial reaction, and cycled 25 times at an annealing temperature of 50° C. Taq DNA Polymerase (Promega, Madison, Wis.) was used in each step. The final 100 μl reaction was gel purified, and the desired hu β1α1 amplification product isolated.

The hu β1α1 insert was ligated with the PCR 2.1 plasmid vector (TA Cloning kit, Invitrogen, Carlsbad, Calif.), and transformed into an INVa'F. bacterial cloning host. PCR colony screening was used to select a single positive colony, from which plasmid DNA was isolated (QIAprep Spin Mini Kit, Qiagen, Inc., Valencia Calif.). Plasmid was cut with NcoI and XhoI restriction enzymes (New England BioLabs Inc., Beverly, Mass.), gel purified, and the hu β1α1 DNA fragment isolated. The hu β1α1 DNA insert was ligated with NcoI/XhoI digested pET-21 d(+) plasmid expression vector (Novagen, Inc., Madison, Wis.), and transformed into BL21(DE3) expression host (Novagen, Inc., Madison, Wis.). Bacterial colonies were selected based on PCR colony and protein expression screening.

Plasmid DNA was isolated from positive colonies (QlAquick Gel Extraction Kit, Qiagen Inc., Valencia, Calif.) and sequenced with the T7 5′-TAATACGACTCACTATAGGG-3′ (SEQ ID NO:32) and T7 terminator←5′-GCTAGTTATTGCTCAGCGG-3′ (SEQ ID NO:33) primers. After sequence verification a single clone was selected for expression of the hu β1α1 peptide (RTL300).

A 30 amino acid huMBP-85-99/peptide linker cartridge was genetically inserted into the “empty” hu β1α1 (RTL300) coding sequence between Arg5 and Pro6 of the β1 chain. The 90 bp DNA sequence encoding peptide-Ag and linker was inserted at position 16 of the RTL300 DNA construct in a three step PCR reaction, using Taq DNA Polymerase (Promega, Madison, Wis.).

In the first step, pET-21d(+)/RTL300 plasmid was used as template in two separate PCR reactions. In the first reaction, the region from the start of the T7 priming site of the pET-21 d(+) plasmid to the point of insertion within the hu β1α1 (RTL300) sequence was amplified with the following primers:

5′-GCTAGTTATTGCTCAGCGG-3′(T7→, SEQ ID NO:33), and

5-AGGCTGCCACAGGAAACGTGGGCCTCCACCTCCAGAGCCTCGGOGCACTAGT GAGCCTCCACCTCCACGCGGGGTAACGATGTTTTTGAAGAAGTGAACAACCGGG TTTTCTCGGGTGTCCCCCATGGTAAT-3′ (huMBP-85-99Lig←, SEQ ID NO:34).

In the second reaction, the region from the point of insertion within the hu β1α1 (RTL300) sequence to the end of the T7-terminator priming site was amplified with the following primers:

5′-CCACGTTTCCTGTGGCAGCC-3′ (huMBP-85-99Lig→, SEQ ID NO:35), and

5′-GCTAGTTATTGCTCAGCGG-3′ (T7terminator←, SEQ ID NO:33).

Each reaction was gel purified, and the desired bands isolated.

In the second step, 5 μl of each purified amplification product was added to a primer free ‘anneal-extend’ PCR reaction mix, and cycled for 5 times at an annealing temperature of 50° C. In the third step, a 50 μl PCR ‘amplification mix’ containing the 5′-TAATACGACTCACTATAGGG-3′ (T7→, SEQ ID NO:32) and 5′-GCTAGTTATTGCTCAGCGG-3′ (T7terminator←, SEQ ID NO:33) primers was then added directly to the ‘anneal-extend’ reaction, and the entire volume cycled 25 times using a 55° C. annealing temperature. The non-complimentary 5′ tail of the huMBP-85-99lig←primer included DNA encoding the entire peptide/linker cartridge, and the region down-stream from the point of insertion.

The resulting amplification product hybridized easily with the PCR product produced in the second reaction, via the complimentary 3′ and 5′ ends of each respectively. DNA polymerase then extended from the 3′-end of each primer, creating the full length hu β1α1/huMBP-85-99 (RTL301) construct, which acted as template in the ‘amplification’ step. The reaction was purified using agarose gel electrophoresis, and the desired hu β1α1/huMBP-85-99 (RTL301) band isolated. The PCR product was then cut with NcoI and XhoI restriction enzymes, gel purified, ligated with a similarly cut pET-21 d(+) plasmid expression vector, and transformed into a BL21(DE3) E. coli expression host. Transformants were screened for protein expression and the presence of the desired insert with a PCR colony screen. Plasmid DNA was isolated from several positive clones and sequenced. A single positive clone was selected for expression of the hu β1α1/huMBP-85-99 peptide (RTL301).

Repeated sequence analysis of pET-21 d(+)/RTL300 and pET-21d(+)/RTL301 plasmid DNA constructs revealed the same thymine to cytosine single base pair deviation at position 358 and position 458 (RTL300 and RTL301 numbering, respectively), than had been reported previously for HLA-DRA*0101 (genebank accession #M60333), which resulted in an F150L mutation in the RTL300 and RTL301 molecules (RTL301 numbering).

Site directed mutagenesis was used to revert the sequence to the Genebank #M60333 sequence. Two PCR reactions were performed using the pET-21d(+)/RTL300 and pET-21d(+)/RTL301 plasmids as template. For RTL300 the primers:

5′-TAATACGACTCACTATAGGG-3′ (T7→, SEQ ID NO:32), and

5′-TCAAAGTCAAACATAAACTCGC-3′ (huBA-F150L←, SEQ ID NO:36) were used.

For RTL301 the primers:

5′-GCGAGTTTATGTTTGACTTTGA-3′ (huBA-F150L→, SEQ ID NO:37), and

5′-GCTAGTTATTGCTCAGCGG-3′ (T7terminator←, SEQ ID NO:33) were used.

The two resulting amplification products were gel purified and isolated (QIAquick gel extraction kit, Qiagen, Valencia, Calif.), annealed, and amplified as described earlier, based on the complimentary 3′ and 5′ ends of each of the PCR products. The final amplification reactions were gel purified, and the desired PCR products isolated. The NcoI and XhoI restriction sites flanking each were then used to subclone the RTL DNA constructs into fresh pET-21d(+) plasmid for transformation into BL21(DE3) competent cells and plasmid sequence verification. Positive clones were chosen for expression of the “empty” HLA-DR2 Blal-derived RTL302 molecule and the MBP-85-99-peptide coupled RTL303 molecule (FIG. 2).

Expression and in vitro Folding of the RTL Constructs

E. coli strain BL21 (DE3) cells were transformed with the pET21 d+/RTL vectors. Bacteria were grown in one liter cultures to mid-logarithmic phase (OD₆₀₀=0.6-0.8) in Luria-Bertani (LB) broth containing carbenicillin (50 μg/ml) at 37 ° C. Recombinant protein production was induced by addition of 0.5 mM isopropyl β-D-thiogalactoside (IPTG). After incubation for 3 hours, the cells were collected by centrifugation and stored at −80 ° C. before processing. All subsequent manipulations of the cells were at 4 ° C. The cell pellets were resuspended in ice-cold PBS, pH 7.4, and sonicated for 4×20 seconds with the cell suspension cooled in a salt/ice/water bath. The cell suspension was then centrifuged, the supernatant fraction was poured off, the cell pellet resuspended and washed three times in PBS and then resuspended in 20 mM ethanolamine/6 M urea, pH 10, for four hours. After centrifugation, the supernatant containing the solubilized recombinant protein of interest was collected and stored at 4 ° C. until purification.

The recombinant proteins of interest were purified and concentrated by FPLC ion-exchange chromatography using Source 30Q anion-exchange media (Pharmacia Biotech, Piscataway, N.J.) in an XK26/20 column (Pharmacia Biotech), using a step gradient with 20 mM ethanolamine/6M urea/1M NaCl, pH 10. The proteins were dialyzed against 20 mM ethanolamine, pH 10.0, which removed the urea and allowed refolding of the recombinant protein. This step was critical. Basic buffers were required for all of the RTL molecular constructs to fold correctly, after which they could be dialyzed into PBS at 4° C. and concentrated by centrifugal ultrafiltration with Centricon-10 membranes (Amicon, Beverly, Mass.). For purification to homogeneity, a finish step was included using size exclusion chromatography on Superdex 75 media (Pharmacia Biotech) in an HR16/50 column (Pharmacia Biotech). The final yield of purified protein varied between 15 and 30 mg/L of bacterial culture.

Circular Dichroism and Thermal Transition Measurements

CD spectra were recorded on a JASCO J-500A spectropolarimeter with an IF-500 digital interface and thermostatically con-trolled quartz cells (Hellma, Mulheim, Germany) of 2, 1, 0.5, 0.1 and 0.05 mm pathlength depending on peptide concentration. Data are presented as mean residue weight ellipticities. Calibration was regularly performed with (+)-10-camphorsulfonic acid (Sigma) to molar ellipticities of 7780 and -16,160 deg. cm²/dmol at 290.5 and 192.5 nm, respectively (Chen et al., 1977). In general, spectra were the average of four to five scans from 260 to 180 m-n recorded at a scanning rate of 5 nm/min with a four second time constant. Data were collected at 0.1 nm intervals. Spectra were averaged and smoothed using the built-in algorithms of the Jasco program and buffer baselines were subtracted. Secondary structure was estimated with the program CONTIN (Provencher et al., 1981). Thermal transition curves were recorded at a fixed wavelength of 222 nm. Temperature gradients from 5 to 90 or 95° C. were generated with a programmer controlled circulating water bath (Lauda PM350 and RCS20D). Heating and cooling rates were between 12 and 18° C./h. Temperature was monitored in the cell with a thermistor and digital thermometer (Omega Engineering), recorded and digitized on an XY plotter (Hβ7090A, Hewlett Packard), and stored on disk. The transition curves were normalized to the fraction of the peptide folded (F) using the standard equation: F=([U]−[U]u)/([U]n −[U]u), where [U]n and [U]u represent the ellipticity values for the fully folded and fully unfolded species, respectively, and [U] is the observed ellipticity at 222 nm.

### Example 7

Homology Modeling

Previous protein engineering studies describing recombinant TCR ligands (RTLs) derived from the alpha-I and beta-I domains of rat MHC class II RT1.B (Burrows et al., 1999). Homology modeling studies of the heterodimeric MHC class II protein HLA-DR2, and specifically, the alpha-I and beta-I segments of the molecule that comprise the antigen binding domain, were conducted based on the crystal structures of human DR (Smith et al., 1998; Li et al., 2000; Brown et al., 1993; Murthy et al., 1997). In the modeling studies described herein, three facets of the source proteins organization and structure were focused on: (1) The interface between the membrane-proximal surface of the beta-sheet platform and the membrane distal surfaces of the alpha-2 and beta-2 Ig-fold domains, (2) the internal hydrogen bonding of the alpha-1 and beta-1 domains that comprise the peptide binding/TCR recognition domain, and (3), the surface of the RTLs that was expected to interact with the TCR.

Side-chain densities for regions that correspond to primary sequence between the beta-I and beta-2 domains of human DR and murine I-EK showed evidence of disorder in the crystal structures (Smith et al., 1998; Li et al., 2000; Brown et al., 1993; Murthy et al., 1997; Fremont et al., 1996), supporting the notion that these serve as linker regions between the two domains with residue side-chains having a high degree of freedom of movement in solution. High resolution crystals of MHC class II DRI and DR2 (Smith et al., 1998; Li et al., 2000; Brown et al., 1993; Murthy et al., 1997) contained a large number of water molecules between the membrane proximal surface of the β-sheet platform and the membrane distal surfaces of the α2 and 132 μg-fold domains. The surface area of interaction between domains was quantified by creating a molecular surface for the β1α1 and a 2β2 Ig-fold domains with an algorithm developed by Michael Connolly (Connolly, 1986) using the crystallographic coordinates for human DR2 available from the Brookhaven Protein Data Base (1BX2). In this algorithm the molecular surfaces are represented by “critical points” describing holes and knobs. Holes (maxima of a shape function) are matched with knobs (minima). The surface areas of the a 1β1 and a 2β2-Ig-folddomains were calculated independently, defined by accessibility to a probe of radius 0.14 nm, about the size of a water molecule. The surface area of the MHC class II a B-heterodimer was 160 nm², while that of the RTL construct was 80 nm² and the α2β2-Ig-fold domains was 90 nm². Approximately 15 nm² (19%) of the RTL surface was buried by the interface with the Ig-fold domains in the MHC class II α β-heterodimer.

Human, rat and murine MHC class II alpha chains share 30% identity and the beta chains share 35% identity. The backbone traces of the structures solved using X-ray crystallography showed strong homology when superimposed, implying an evolutionarily conserved structural motif. The variability between the molecules is primarily within the residues that delineate the peptide-binding groove, with side-chain substitutions designed to allow differential antigenic-peptide binding. The α1 and β1 domains of HLA-DR showed an extensive hydrogen-bonding network and a tightly packed and buried hydrophobic core. This tertiary structure appears similar to the molecular interactions that provide structural integrity and thermodynamic stability to the alpha-helix/beta-sheet scaffold characteristic of scorpion toxins (Zhao et al., 1992; Housset et al., 1994; Zinn-Justin et al., 1996). The β1-domain of MHC class II molecules contains a disulfide bond that covalently couples the carboxyl-terminal end to the first strand of the anti-parallel β-sheet platform contributed by the β1-domain. This structure is conserved among MHC class II molecules from rat, human and mouse, and is conserved within the α2 domain of MHC class I. It appears to serve a critical function, acting as a “linchpin” that allows primary sequence diversity in the molecule while maintaining its tertiary structure. Additionally, a “network” of conserved aromatic side chains (30) appear to stabilize the RTLs. The studies described herein demonstrate that the antigen binding domain remains stable in the absence of the α2 and β2 Ig-fold domains.

### Example 8

### Expression and Production of RTLs

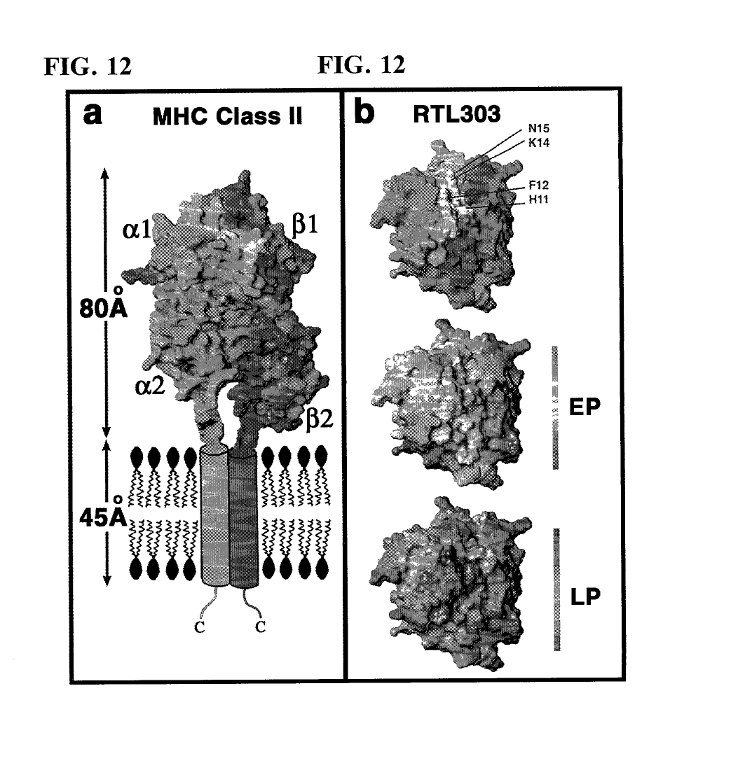

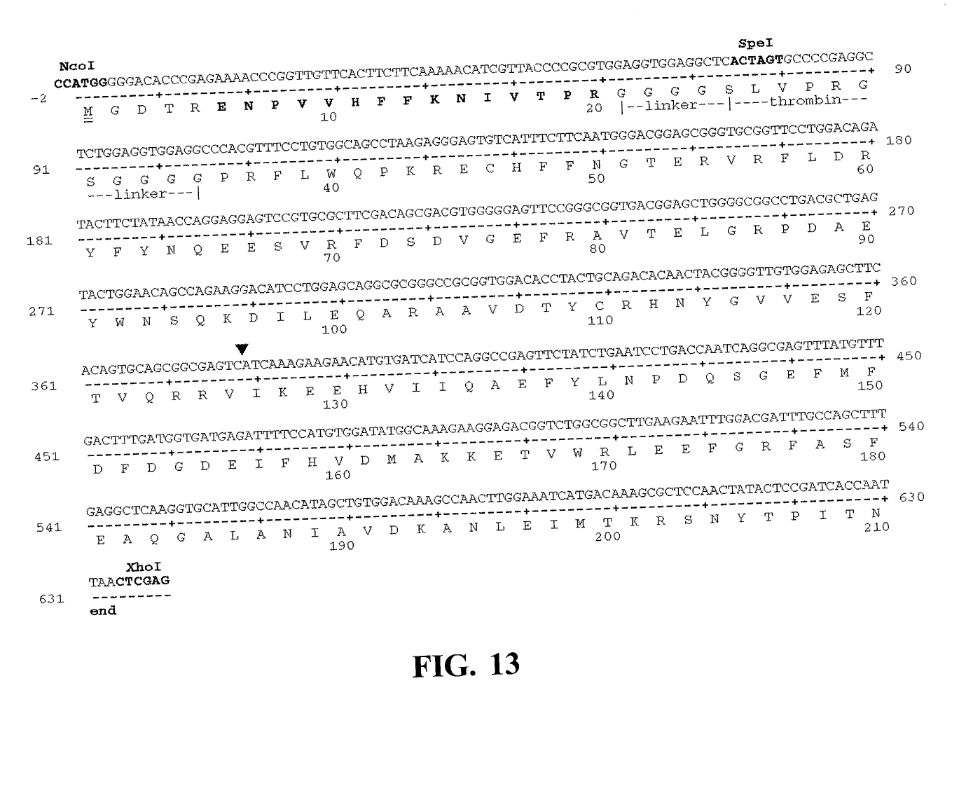

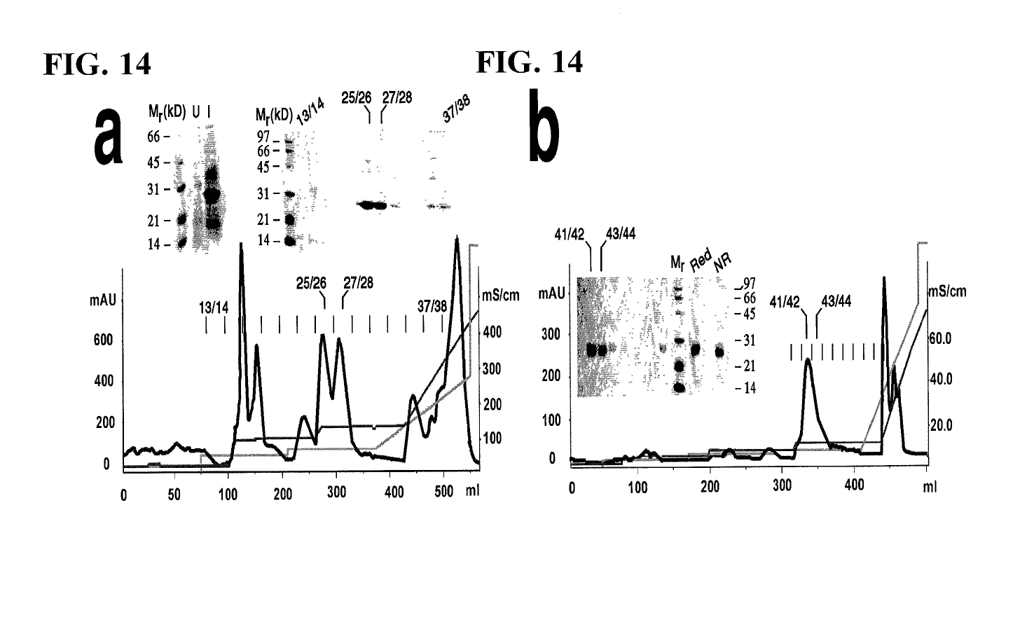

Novel genes were constructed by splicing sequence encoding the amino terminus of HLA-DR2 alpha-1 domain to sequence encoding the carboxyl terminus of the beta-1 domain. The nomenclature RTL (“recombinant TCR ligand”) was used for proteins with this design (see U.S. patent Ser. No. 09/153,586). In the studies described herein, experiments are presented that used the “empty” RTL with the native sequence (RTL302), a covalent construct that contained the human MBP-85-99 antigenic peptide (RTL303), and versions of these molecules (RTL300, “empty”; RTL301, containing MBP-85-99) that had a single phenylalanine to leucine alteration (F150L, RTL303 numbering) that eliminated biological activity (See FIG. 13; Table III). Earlier work had demonstrated that the greatest yield of material could be readily obtained from bacterial inclusion bodies, refolding the protein after solubilization and purification in buffers containing 6M urea (Burrows et al., 1999). Purification of the RTLs was straightforward and included ion exchange chromatography followed by size exclusion chromatography (FIG. 14).

After purification, the protein was dialyzed against 20 mM ethanolamine, pH 10.0, which removed the urea and allowed refolding of the recombinant protein. This step was critical. Basic buffers were required for all of the RTL molecular constructs to fold correctly, after which they could be dialyzed into PBS at 4° C. for in vivo studies. The final yields of “empty” and antigenic peptide-coupled RTLs was approximately 15-30 mg/liter culture.

### Example 9

### Biochemical Characterization and Structural Analysis of Human RTLs

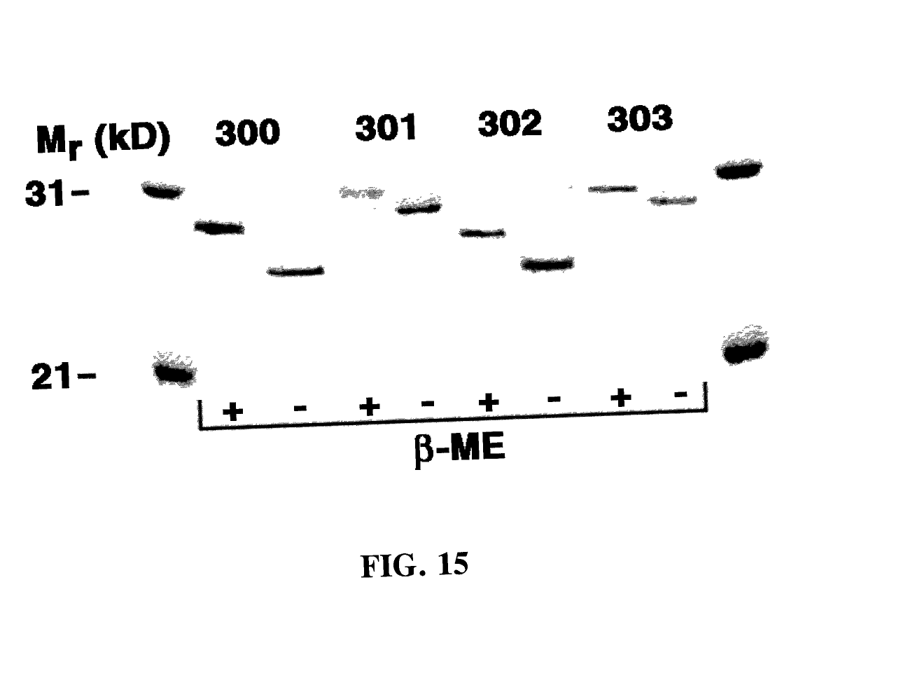

Oxidation of cysteines 46 and 110 (RTL303 amino acid numbering, corresponding to DR2 beta chain residues 15 and 79) to reconstitute the native disulfide bond was demonstrated by a gel shift assay (FIG. 15), in which identical samples with or without the reducing agent β-mercaptoeth-anol (β-ME) were boiled 5 minutes prior to SDS-PAGE. In the absence of β-ME disulfide bonds are retained and proteins typically demonstrate a higher mobility during electrophoresis through acrylamide gels due to their more compact structure. Representative examples of this analysis are shown for the “empty” RTL300 and RTL302, and the MBP-coupled RTL301 and RTL303 molecules (FIG. 15). All of the RTL molecules produced showed this pattern, indicating presence of the native conserved disulfide bond. These data represent a confirmation of the conformational integrity of the molecules.

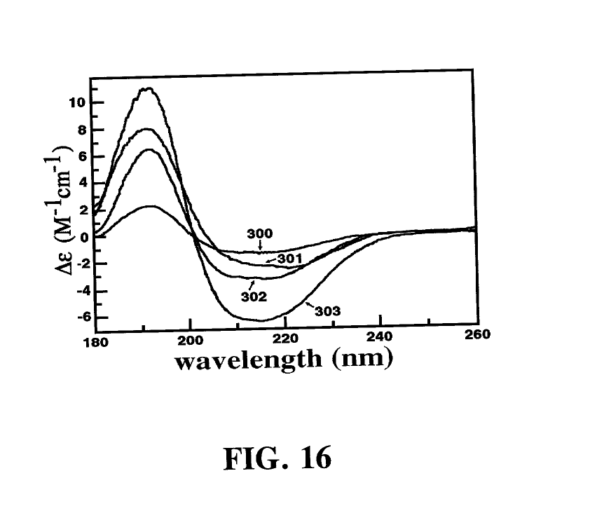

Circular dichroism (CD) demonstrated the highly ordered secondary structures of RTL 302 and RTL303 (FIG. 16; Table I). RTL303 contained approximately 38% alpha-helix, 33% beta-strand, and 29% random coil structures. Comparison with the secondary structures of class II molecules determined by x-ray crystallography (Smith et al., 1998; Li et al., 2000; Brown et al., 1993; Murthy et al., 1997; Fremont et al., 1996) provided strong evidence that RTL303 shared the beta-sheet platform/anti-parallel alpha-helix secondary structure common to all class II antigen binding domains (Table 4, FIG. 16).

TABLE 4
Secondary structure analysis of RTLs and MHC class II β-1/α-1 domains.

| Molecule | description | α-helix | β-sheet^{c} | other | total | Reference |
|---|---|---|---|---|---|---|
| RTL201 | RT1.B β1α1/Gp-MBP72-89 | 0.28 | 0.39 | 0.33 | 1.0 | Burrows et al., 1999 |
| RTL300 | DR2 β1α1(F150L)a | — | — | — | ND^{b} | Chang et al., 2001 |
| RTL301 | DR2 β1α1/hu-MBP85-99(F150L) | 0.20 | 0.35 | 0.46 | 1.0 | Chang et al., 2001 |
| RTL302 | DR2 β1α1(empty) | 0.26 | 0.31 | 0.43 | 1.0 | Chang et al., 2001 |
| RTL303 | DR2 β1α1/hu-MBP85-99 | 0.38 | 0.33 | 0.29 | 1.0 | Chang et al., 2001 |
| 1BX2 | DR2 (DRA*0101, DRB1*1501) | 0.32 | 0.37 | 0.31 | 1.0 | Smith et al., 1998 |
| 1AQD | DR1 (DRA*0101, DRB1 0101) | 0.32 | 0.37 | 0.31 | 1.0 | Murthy et al., 1997 |
| 1IAK | murine I-A^{k} | 0.34 | 0.37 | 0.29 | 1.0 | Fremont et al., 1996 |
| 1IEA | murine I-E^{k} | 0.27 | 0.31 | 0.42 | 1.0 | Fremont et al., 1996 |

^{a}F150L based on RTL303 numbering (See FIG. 2).
^{b}RTL300 CD data could not be fit using the variable selection method.
^{c}β-sheet includes parallel and anti-parallel β-sheet and β-turn structures.

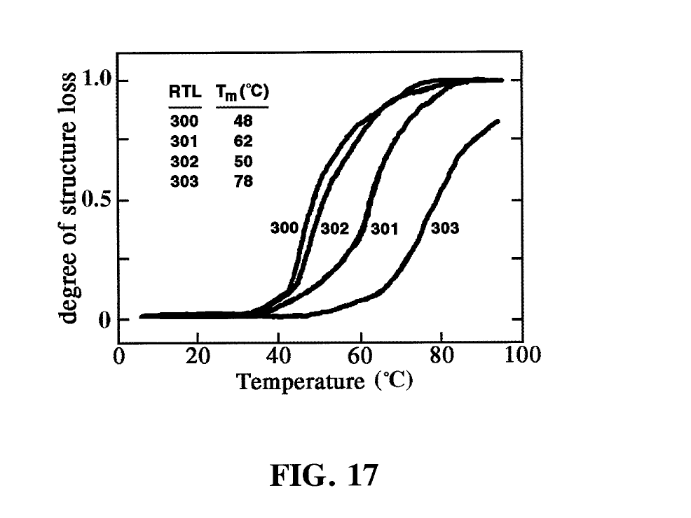

Structure loss upon thermal denaturation indicated that the RTLs used in this study are cooperatively folded (FIG. 17). The temperature (Tm) at which half of the structure is lost for RTL303 is approximately 78° C., which is similar to that determined for the rat RT1.B MHC class II-derived RTL201 (Burrows et al., 1999). RTL302, which does not contain the covalently coupled Ag-peptide showed a 32% decease in alpha-helical content compared to RTL303 (Table 4). This decrease in helix content was accompanied by a decrease in thermal stability of 36% (28° C.) compared to RTL303, demonstrating the stabilization of the RTL molecule, and by inference, the antigen-presentation platform of MHC class II molecules, that accompanies peptide binding. Again, this trend is similar to what has been observed using rat RTL molecules (Burrows et al., 1999), although the stabilization contributed by the covalently coupled peptide is approximately 3-fold greater for the human RTLs compared to rat RTLs.

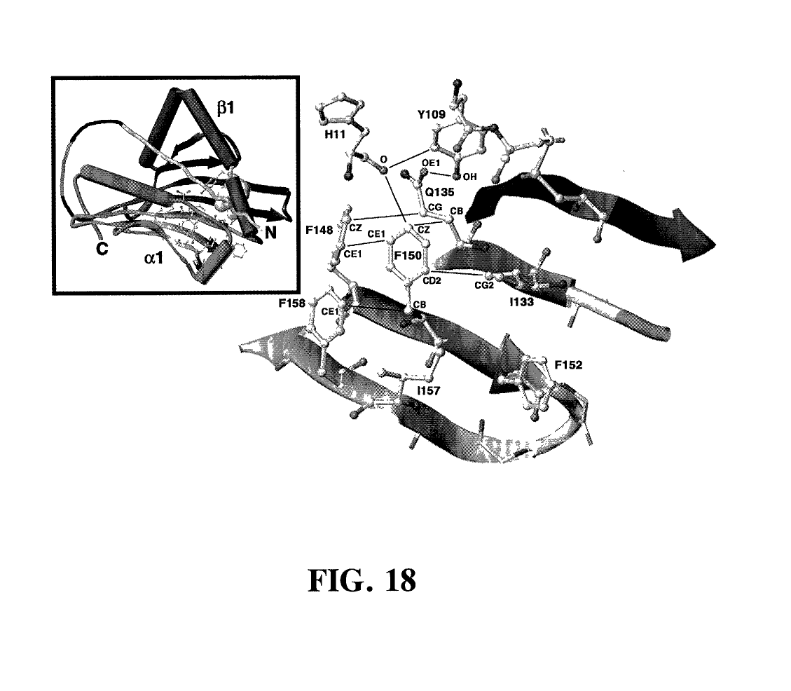

The F150L modified RTL301 molecule showed a 48% decrease in alpha-helical content (Table 4) and a 21% (16° C.) decrease in thermal stability compared to RTL303. RTL300, which had the F 50L modification and lacked the covalently-coupled Ag-peptide, showed cooperativity during structure loss in thermal denaturation studies, but was extremely unstable (Tₘ=48° C.) relative to RTL302 and RTL303, and the secondary structure could not be determined from the CD data (FIGS. 16, 17; Table 4). An explanation for the thermal stability data comes from molecular modeling studies using the coordinates from DR2a and DR2b MHC class II crystal structures (PDB accession codes 1FV1 and 1BX2; Smith et al., 1998; Li et al., 2000). These studies demonstrated that F150 is a central residue within the hydrophobic core of the RTL structure (FIG. 18), part of a conserved network of aromatic side chains that appears to stabilize the secondary structure motif that is completely conserved in human class II molecules and is highly conserved between rat, mouse and human MHC class II.

TABLE 5
Interactions of residues within 4Å of F150^{a}

| atom 1 ID | atom 2 ID | distance (Å) |
|---|---|---|
| I133.CG2 (A:I7)^{b} | F150.CD2 (A:F24) | 3.75 |
| I133.CG2 | F150.CE2 | 3.75 |
| Q135.CB (A:Q9) | F150.CE1 | 3.65 |
| Q135.CG | F148.CZ (A:F22) | 4.06 |
| Q135.OE1 | Y109.OH (B:Y78) | 2.49 |
| F148.CE1 | F150.CE1 | 4.07 |
| F150.CB | F158.CE1 (A:F32) | 3.64 |
| F150.CZ | H11.O (C:H90) | 3.77 |
| Y109.CE1 | H11.O | 3.12 |

^{a}F150 (RTL303 numbering) is F24 of the beta chain of DR2. The distances were calculated using coordinates from 1BX2 (Smith et al., 1998).
^{b}The residue are numbered as shown in FIG. 7, with the 1BX2 residue number in parenthesis. For example, F150.CE2 is equivelent to B:F24.CE2; atom CE2 of residue F24 on chain B of the heterodimeric 1BX2 crystal structure. Chain C is the bound antigenic peptide.

The motif couples three anti-parallel beta-sheet strands to a central unstructured stretch of polypeptide between two alpha-helical segments of the alpha-1 domain. The structural motif is located within the alpha-1 domain and “caps” the alpha-l domain side at the end of the peptide binding groove where the amino-terminus of the bound Ag-peptide emerges.

Thus, soluble single-chain RTL molecules have been constructed derived from the antigen-binding β1 and α1 domains of human MHC class II molecule DR2. The RTLs lack the α2 domain, the β2 domain known to bind to CD4, and the transmembrane and intra-cytoplasmic sequences. The reduced size of the RTLs gave us the ability to express and purify the molecules from bacterial inclusion bodies in high yield (15-30 mg/L cell culture). The RTLs refolded upon dialysis into PBS and had excellent solubility in aqueous buffers.

The data presented herein demonstrate clearly that the human DR2-derived RTL302 and RTL303 retain structural and conformational integrity consistent with crystallographic data regarding the native MHC class II structure. MHC class II molecules form a stable heterodimer that binds and presents antigenic peptides to the appropriate T cell receptor (FIG. 12). While there is substantial structural and theoretical evidence to support this model (Brown et al., 1993; Murthy et al., 1997; Fremont et al., 1996; Ploegh et al., 1993; Schafer et al., 1995), the precise role that contextual information provided by the MHC class II molecule plays in antigen presentation, T cell recognition and T cell activation remains to be elucidated. The approach described herein used rational protein engineering to combine structural information from X-ray crystallographic data with recombinant DNA technology to design and produce single chain TCR ligands based on the natural MHC class II peptide binding/T cell recognition domain. In the native molecule this domain is derived from portions of the alpha and beta polypeptide chains which fold together to form a tertiary structure, most simply described as a beta-sheet platform upon which two anti-parallel helical segments interact to form an antigen-binding groove. A similar structure is formed by a single exon encoding the alpha-1 and alpha-2 domains of MHC class I molecules, with the exception that the peptide-binding groove of MHC class II is open-ended, allowing the engineering of single-exon constructs that incorporate the peptide binding/T cell recognition domain and an antigenic peptide ligand (Kozono et al., 1994).

From a drug engineering and design perspective this prototypic molecule represents a major breakthrough. Development of the human RTL molecules described herein separates the peptide binding (1β1 domains from the platform (2β2 Ig-fold domains, allowing studies of their biochemical and biological properties independently, both from each other and from the vast network of information exchange that occurs at the cell surface interface between APC and T cell during MHC/peptide engagement with the T cell receptor. Development of human RTL molecules described herein allows careful evaluation of the specific role played by a natural TCR ligand independent from the platform (2β2 Ig-fold domains of MHC class II).

When incubated with peptide specific Th1 cell clones in the absence of APC or costimulatory molecules, RTL303 initiated a subset of quantifiable signal transduction processes through the TCR. These included rapid ζ chain phosphorylation, calcium mobilization, and reduced ERK kinase activity, as well as IL-10 production. Addition of RTL303 alone did not induce proliferation. T cell clones pretreated with cognate RTLs prior to restimulation with APC and peptide had a diminished capacity to proliferate and secrete IL-2, and secreted less IFN-γ (Importantly, IL-10 production persisted (see below). These data elucidate for the first time the early signaling events induced by direct engagement of the external TCR interface, in the absence of signals supplied by co-activation molecules.

Modeling studies have highlighted a number of interesting features regarding the interface between the β1α1and α2β2-Ig-fold domains. The α1 and β1 domains showed an extensive hydrogen-bonding network and a tightly packed and buried hydrophobic core. The RTL molecules, composed of the α1 and β1 domains may have the ability to move as a single entity independent from the α2β2-Ig-fold “platform”. Without being bound by theory, flexibility at this interface may be required for freedom of movement within the α1 and β1 domains for binding/exchange of peptide antigen. Alternatively or in combination, this interaction surface may play a potential role in communicating information about the MHC class II/peptide molecules interaction with TCRs back to the APC.

Critical analysis of the primary sequence of amino acid residues within two helical turns (7.2 residues) of the conserved cysteine 110 (RTL303 numbering) as well as analysis of the β-sheet platform around the conserved cysteine 46 (RTL303 numbering) reveal a number of interesting features of the molecule, the most significant being very high diversity along the peptide-binding groove face of the helix and β-sheet platform. Interestingly, the surface exposed face of the helix composed of residues L99, E100, R103, A104, D107, R111, and Y114 (FIG. 1) is conserved in all rat, human and mouse class II and may serve an as yet undefined function.

Cooperative processes are extremely common in biochemical systems. The reversible transformation between an alpha-helix and a random coil conformation is easily quantified by circular dicroism. Once a helix is started, additional turns form rapidly until the helix is complete. Likewise, once it begins to unfold it tends to unfold completely. A normalized plot of absorption of circularly polarized light at 222 nm versus temperature (melting curve) was used to define a critical Tₘ for each RTL molecule. The melting temperature was defined as the midpoint of the decrease in structure loss calculated from the loss of absorption of polarized light at 222 um. Because of their size and biochemical stability, RTLs will serve as a platform technology for development of protein drugs with engineered specificity for particular target cells and tissues.

### Example 10

### TCR Signaling: Rational, Materials, and Methods

Development of a minimal TCR ligand allows study of TCR signaling in primary T cells and T cell clones in the absence of costimulatory interactions that complicate dissection of the information cascade initiated by MHC/peptide binding to the TCR alpha and beta chains. A minimum “T cell receptor ligand” conceptually consists of the surface of an MHC molecule that interacts with the TCR and the 3 to 5 amino acid residues within a peptide bound in the groove of the MHC molecule that are exposed to solvent, facing outward for interaction with the TCR. The biochemistry and biophysical characterization of Recombinant TCR Ligands (RTLs) derived from MHC class II are described above, such as the use of the α-1 and β-1 domains of HLA-DR2 as a single exon of approximately 200 amino acid residues with various amino-terminal extensions containing antigenic peptides. These HLA-DR2-derived RTLs fold to form the peptide binding/T cell recognition domain of the native MHC class II molecule.

Inflammatory Th1, CD4+ T cells are activated in a multi-step process that is initiated by co-ligation of the TCR and CD4 with MHC/peptide complex present on APCs. This primary, antigen-specific signal needs to be presented in the proper context, which is provided by co-stimulation through interactions of additional T cell surface molecules such as CD28 with their respective conjugate on APCs. Stimulation through the TCR in the absence of co-stimulation, rather than being a neutral event, can induce a range of cellular responses from full activation to anergy or cell death (Quill et al., 1984). As described herein Ag-specific RTLs were used induce a variety of human T cell signal transduction processes as well as modulate effector functions, including cytokine profiles and proliferative potential.

Recombinant TCR Ligands

Recombinant TCR Ligands were produced as described above.

Synthetic Peptides.

MBβ85-99 peptide (ENPVVHFFKNIVTPR, SEQ ID NO:38) and “CABL”, BCR-ABL b3α2 peptide (ATGFKQSSKALQRPVAS, SEQ ID NO:39) (ten Bosch et al., 1995) were prepared on an Applied Biosystems 432A (Foster City, Calif.) peptide synthesizer using finoc solid phase synthesis. The MBP peptide was numbered according to the bovine MBP sequence (Martenson, 1984). Peptides were prepared with carboxy terminal amide groups and cleaved using thianisole/1,2-ethanedithiol/dH 2 0 in trifluoroacetic acid (TFA) for 1.5 hours at room temperature with gentle shaking. Cleaved peptides were precipitated with 6 washes in 100% cold tert-butylmethyl ether, lyophilized, and stored at −70 ° C. under nitrogen. The purity of peptides was verified by reverse phase HPLC on an analytical Vydac C18 column.

T Cell Clones.

Peptide-specific T cell clones were selected from peripheral blood mononuclear cells (PBMC) of a multiple sclerosis (MS) patient homozygous for HLA-DRB 1*1501 and an MS patient homozygous for HLA-DRB1*07, as determined by standard serological methods and further confirmed by PCR amplification with sequence-specific primers (PCR-SSP) (Olerup et al., 1992). Frequencies of T cells specific for human MBP85-99 and CABL were determined by limiting dilution assay (LDA). PBMC were prepared by ficoll gradient centrifugation and cultured with 10 μg/ml of either MBP85-99 or CABL peptide at 50,000 PBMC/well of a 96-well U-bottomed plate plus 150,000 irradiated (2500 rad) PBMC/well as antigen-presenting cells (APCs) in 0.2 ml medium (RPMI 1640 with 1% human pooled AB serum, 2 mM L-glutamine, 1 mM sodium pyruvate, 100 μg/ml penicillin G, and 100 μg/ml streptomycin) for 5 days, followed by adding 5 ng/ml IL-2 (R & D Systems, Minneapolis, Minn.) twice per week. After three weeks, the culture plates were examined for cellular aggregation or “clump formation” by visual microscopy and the cells from the “best” 20-30 clump-forming wells among a total of 200 wells per each peptide Ag were expanded in 5 ng/ml IL-2 for another 1-2 weeks. These cells were evaluated for peptide specificity by the proliferation assay, in which 50,000 T cells/well (washed 3×) were incubated in triplicate with 150,000 freshly isolated and irradiated APC/well plus either medium alone, 10 mg/ml MBβ85-99 or 10 mg/ml CABL pep-tide for three days, with 3H-Tdy added for the last 18 hours. Stimulation index (S.I.) was calculated by dividing the mean CPM of peptide-added wells by the mean CPM of the medium alone control wells. T cell isolates with the highest S.I. for a particular peptide antigen were selected and expanded in medium containing 5 ng/ml IL-2, with survival of 1-6 months, depending on the clone, without further stimulations.

Sub-Cloning and Expansion of T Cell Number.

Selected peptide-specific T cell isolates were sub-cloned by limiting dilution at 0.5 T cells/well plus 100,000 APC/well in 0.2 ml medium containing 10 ng/ml anti-CD3 (Pharmingen, San Diego, Calif.) for three days, followed by addition of 5 ng/ml IL-2 twice per week for 1-3 weeks. All wells with growing T cells were screened for peptide-specific response by the proliferation assay and the well with the highest S.I. was selected and continuously cultured in medium plus IL-2. The clonality of cells was determined by RT-PCR, with a clone defined as a T cell population utilizing a single TCR V a gene. T cell clones were expanded by stimulation with 10 ng/ml anti-CD3 in the presence of 5×10⁶ irradiated (4500 rad) EBV-transformed B cell lines and 25×10⁶ irradiated (2500 rad) autologous APC per 25 cm 2 flask in 10% AB pooled serum (Bio-Whittaker, MD) for 5 days, followed by washing and resuspending the cells in medium containing 5 ng/ml IL-2, with fresh IL-2 additions twice/week. Expanded T cells were evaluated for peptide-specific proliferation and the selected, expanded T cell clone with the highest proliferation S.I. was used for experimental procedures.

Cytokine detection by ELISA. Cell culture supernatants were recovered at 72 hours and frozen at −80 ° C. until use. Cytokine measurement was performed by ELI SA as previously described (Bebo et al., 1999) using cytokine specific capture and detection antibodies for IL-2, IFN-γ, IL-4 and IL-10 (Pharmingen, San Diego, Calif.). Standard curves for each assay were generated using recombinant cytokines (Pharmingen), and the cytokine concentration in the cell supernatants was determined by interpolation.

Flow Cytometry.

Two color immunofluorescent analysis was performed on a FACScan instrument (Becton Dickinson, Mountain View, Calif.) using CellQuest TM software. Quadrants were defined using isotype matched control Abs.

Phosphotyrosine Assay.

T cells were harvested from culture by centrifuging at 400× g for 10 min, washed, and resuspended in fresh RPMI. Cells were treated with RTLs at 20 μM final concentration for various amounts of time at 37° C. Treatment was stopped by addition of ice-cold RPMI, and cells collected by centrifugation. The supernatant was decanted and lysis buffer (50 mM Tris pH 7.5, 150 mM NaCl, 1% NP-40, 0.5% deoxycholate, 0.1% SDS, 1 mM AEBSF [4-(2-aminoethyl) benzenesulfonylfluoride,HCl], 0.8 μM aprotinin, 50 μM bestatin, 20 μM leupeptin, 10 μM pepstatin A, 1 mM activated sodium orthovanadate, 50 mM NaF, 0.25 mM bpV [potassium bisperoxo(1,10-phenanthroline) oxovanadate], 50 μM Phenylarsine Oxide) was added immediately. After mixing at 4° C. for 15 min to dissolve the cells, the samples were centrifuged for 15 min and cell lysate was collected, to which was added an equal volume of sample loading buffer, mixed, and boiled for 5 min and then aliquots separated by 15% SDS-PAGE. Protein was transferred to PVDF membrane for western blot analysis. Western blot block buffer: 10 mM Tris-HCl (pH 7.5), 100 mM NaCl, 0.1% Tween-20, 1% BSA. Primary antibody: anti-phosphotyrosine, clone 4G10, (Upstate Biotechnology, Lake Placid, N.Y.). Secondary and tertiary antibody from ECF Western blot kit (Amersham, Picataway, N.J.). The dried blot was scanned using a Storm 840 scanner (Molecular Dynamics, Sunnyvale, Calif.) and chemifluorescence quantified using ImageQuant version 5.01 (Molecular Dynamics).

ERK Activation Assay.

T cells were harvested and treated with RTLs as for 4 phosphotyrosine assay. Western blot analysis was performed using anti phosph-ERK (Promega, Madison Wis.) at 1:5000 dilution or anti-ERK kinase (New England Biolabs, Beverly, Mass.) at 1:1500 dilution and visualized using ECF Western Blotting Kit. Bands of interest were quantified as described for ζ phosphotyrosine assay.

Cα2+ Imaging.

Human T cells were plated on polylysine-coated 35 mm glass bottom dishes and cultured for 12-24 hr in medium containing IL-2. Fura-2 μM (5 mM) (Molecular Probes) dissolved in the culture medium was loaded on the cells for 30 min in CO2 incubator. After rinse of fura-2 and additional 15 min incubation in the culture medium, the cells were used for calcium measurement. Fluorescent images were observed by an upright microscope (Axioskop FS, Zeiss) with a water immersion objective (UmplanFL 60×/0.8, Olympus). Two wavelengths of the excitation UV light (340 nl or 380 nm) switched by a monochromator (Polychrome 2, Till Photonics) were exposed for 73 msec at 6 seconds interval. The intensity of 380 nm UV light was attenuated by a balancing filter (UG11, OMEGA Optical). The excitation UV light was reflected by a dichroic mirror (FT 395 nm, Carl Zeiss) and the fluorescent image was band-passed (Bβ500-530, Carl Zeiss), amplified by an image intensifier (C7039-02, Hamamatsu Photonics) and exposed to multiple format cooled CCD camera (C4880, Hamamatsu Photonics). The UV light exposure, CCD control, image sampling and acquisition were done with a digital imaging system (ARGUS HiSCA, Hamamatsu Photonics). The background fluorescence was subtracted by the imaging system. During the recording, cells were kept in a culture medium maintained at 30° C. by a stage heater (DTC-200, Dia Medical). The volume and timing of drug application were regulated by a trigger-driven superfusion system (DAD-12, ALA Scientific instruments).

### Example 11

### The Effect of Human RTLs on Human T Cell Clones

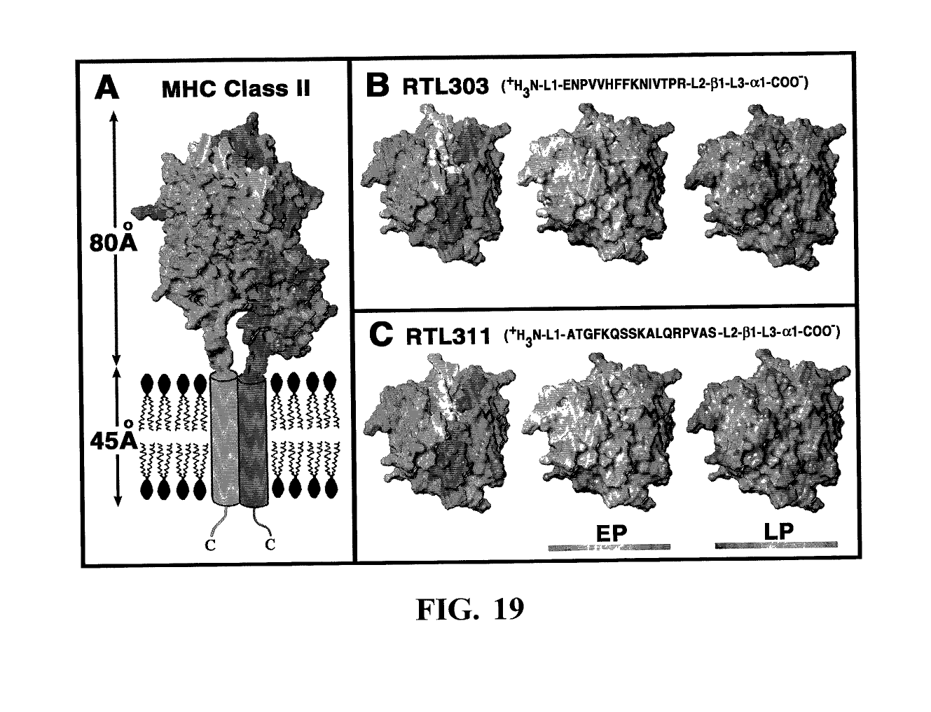

Two different MHC class II DR2-derived RTLs (HLA-DR2b: DRA*0101, DRB1*1501) were used in this study (FIG. 19). RTL303 (Hal/MBβ85-99) and RTL311 (β1α1/CABL) differ only in the antigen genetically encoded at the amino terminal of the single exon RTL. The MBβ85-99 peptide represents the immuno-dominant MBP determinant in DR2 patients (Martin et al., 1992) and the C-ABL peptide (ten Bosch et al., 1995) contains the appropriate motif for binding DR2. The human T cell clones used in this study were selected from a DR2 homozygous patient and a DR7 homozygous MS patient.

Structure-based homology modeling was performed using the refined crystallographic coordinates of human DR2 (Smith et al., 1998) as well as DRI (Brown et al., 1993; Murthy et al., 1997), murine I-E^{k} molecules (Fremont et al., 1996), and scorpion toxins (Zhao et al., 1992). Because a number of amino acid residues in human DR2 (PDB accession number 1BX2) were missing/not located in the crystallographic data (Smith et al., 1998), the correct side chains based on the sequence of DR2 were substituted in the sequence and the peptide backbone was modeled as a rigid body during structural refinement using local energy minimization. These relatively small (approx. 200 amino acid residues) RTLs were produced in Escherichia co/i in large quantities and refolded from inclusion bodies, with a final yield of purified protein between 15-30 mg/L of bacterial culture (Burrows et al., J. Biol. Chem., 2001, accepted). FIG. 19 shows a schematic scale model of an MHC class II molecule on the surface of an APC (FIG. 19A). The HLA-DR2 β1α1-derived RTL303 molecule containing covalently coupled MBP-85-99 peptide (FIG. 19B, left) and the HLA-DR2 β1α1-derived RTL311 molecule containing covalently coupled CABL peptide (FIG. 19C, left), are shown in FIG. 19A with the primary TCR contact residues labeled. The P2 His, P3 Phe, and P5 Lys residues derived from the MBP peptide are prominent, solvent exposed residues. These residues are known to be important for TCR recognition of the MBP peptide. The corresponding residues in the C-ABL peptide (P2 Thr, P3 Gly, P5 Lys) are also shown. Immediately striking is the percentage of surface area that is homologous across species. When shaded according to electrostatic potential (EP) (Connolly, 1983) (FIG. 19B, 19C, middle), or according to lipophilic potential (LP) (Heiden et al., 1993) (FIGS. 19B, 19C, right), subtleties between the molecules are resolved that likely play a specific role in allowing TCR recognition of antigen in the context of the DR2-derived RTL surface.

The design of the constructs allows for substitution of sequences encoding different antigenic peptides using restriction enzyme digestion and ligation of the constructs. Structural characterization using circular dichroism demonstrated that these molecules retained the anti-parallel beta-sheet platform and antiparallel alpha-helices observed in the native class II heterodimer, and the molecules exhibited a cooperative two-state thermal unfolding transition (Burrows et al., J. Biol. Chem., 2001, accepted). The RTLs with the covalently-linked Ag-peptide showed increased stability to thermal unfolding relative to “empty” RTLs, similar to what was observed for rat RT1.B RTLs.

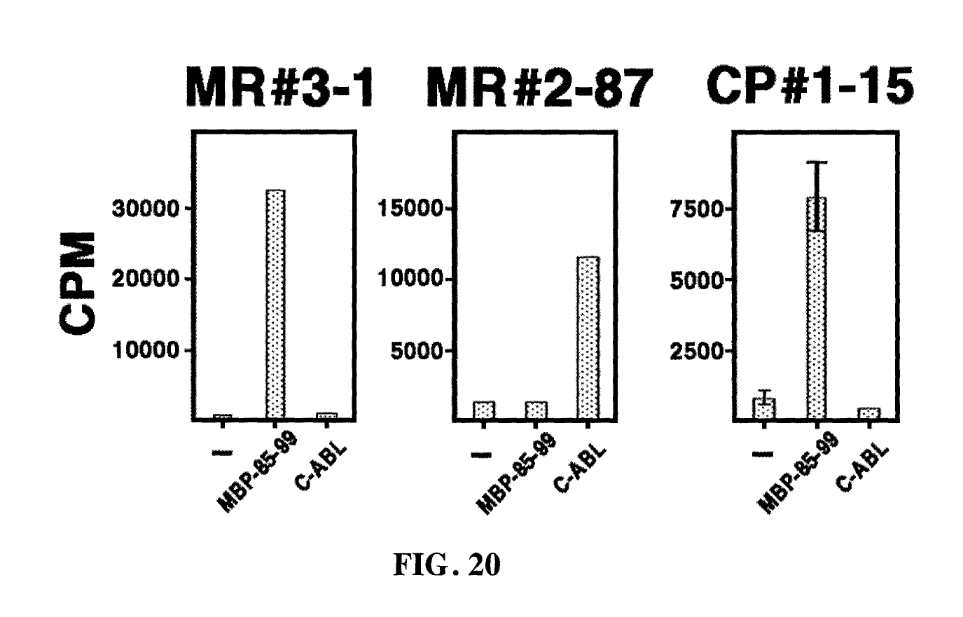

DR2 and DR7 homozygous donor-derived Ag-specific T cell clones expressing a single TCR BV gene were used to evaluate the ability of Ag-specific RTLs to directly modify the behavior of T cells. Clonality was verified by TCR BV gene expression, and each of the clones proliferated only when stimulated by specific peptide presented by autologous APC. DR2 homozygous T cell clone MR#3-1 was specific for the MBβ85-99 peptide and DR2 homozygous clone MR#2-87 was specific for the CABL peptide. The DR7 homozygous T cell clone CP#l-15 was specific for the MBβ85-99 peptide (FIG. 20).

### Example 12

### RTL Treatment Induced Early Signal Transduction Events

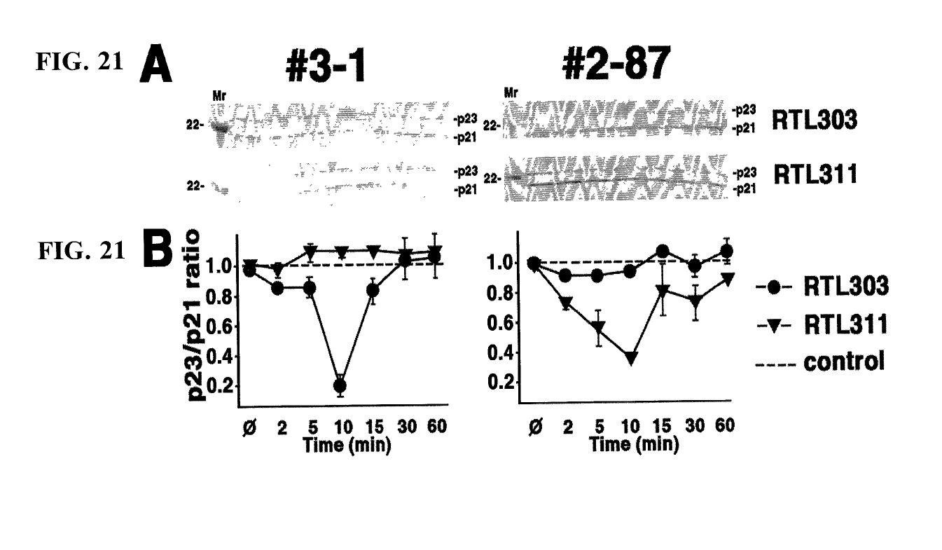

We examined ζ chain phosphorylation in the DR2 homozygous T cell clones MR#3-1 and MR#2-87. MR#3-1 is specific for the MBβ85-99 peptide carried by RTL303, and MR#2-87 is specific for the CABL peptide carried by RTL311. The antigenic peptide on the amino terminal end of the RTLs are the only difference between the two molecules. The TCR-ζ chain is constitutively phosphorylated in resting T cells, and changes in levels of ζ chain phosphorylation are one of the earliest indicators of information processing through the TCR. In resting clones, ζ was phosphorylated as a pair of phospho-protein species of 21 and 23 kD, termed p21 and p23, respectively. Treatment of clone MR#3-1 with 20 pM RTL303 showed a distinct change in the p23/p21 ratio that reached a minimum at 10 minutes (FIG. 21). This same distinct change in the p23/p21 ratio was observed for clone MR#2-87 when treated with 20 μM RTL311 (FIG. 21). Only RTLs containing the peptide for which the clones were specific induced this type of ζ-phosphorylation, previously observed after T cell activation by antagonist ligands (27, 28).

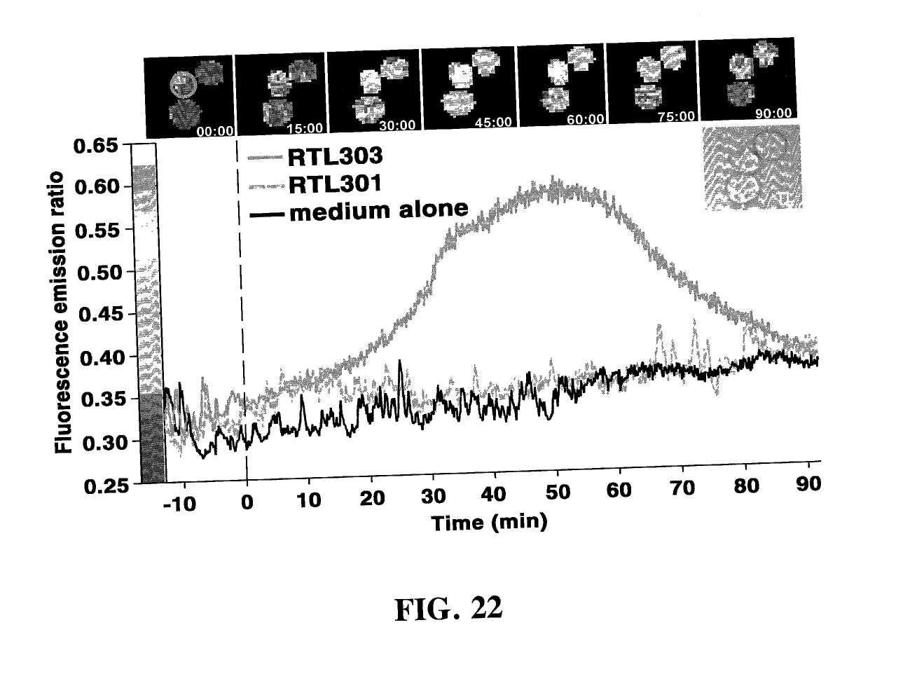

Calcium levels were monitored in the DR2 homozygous T cell clone MR#3-1 specific for the MBβ85-99 peptide using single cell analysis. While there is a general agreement that calcium mobilization is a specific consequence of T cell activation, the pattern of response and dosage required for full activation remain controversial (Wutlfing et al., 1997). It appears that four general patterns of intra-cellular calcium mobilization occur with only the most robust correlating with full T cell proliferation. RTL303 treatment induced a sustained high calcium signal, whereas RTL301 (identical to RTL303 except a single point mutation that altered folding properties, F150L) showed no increase in calcium signal over the same time period (FIG. 22).

RTL effects were further evaluated on levels of the extracellular regulated protein kinase ERK, a key component within the Ras signaling pathway known to be involved in the control of T cell growth and differentiation (Li et al., 1996). The activated form of ERK kinase is itself phosphorylated (Schaeffer et al., 1999), and thus a straightforward measure of ERK activity was to compare the fraction of ERK that is phosphorylated (ERK-P) relative to the total cellular ERK present (T-ERK). Within 15 min after treatment with RTLs, the level of ERK-P was drastically reduced in an Ag-specific fashion. 20,M RTL303 reduced ERK-P by 80% in clone #3-1 and 20 μM RTL311 reduced ERK-P by 90% in clone #2-87 (FIG. 23).

The early signal transduction events that were altered by Ag-specific RTL treatment on the cognate T cell clones led us to investigate the effect of RTL treatment on cell surface markers, proliferation and cytokines. Cell surface expression levels of CD25, CD69 and CD134 (OX40) were analyzed by multicolor flow cytometry at 24 and 48 hr after treatment with RTLs and compared to APC/peptide or Con A stimulated cells. CD69 (32) was already very high (−80% positive) in these clones. APC/peptide induced Ag-specific increases in both CD25 (Kyle et al., 1989) and CD134 (Weinberg et al., 1996) that peaked between 48 and 72 hours (data not shown), while RTL treatment had no effect on these cell surface markers. RTL treatment induced only subtle increases in apoptotic changes as quantified using Annexin V staining and these were not Ag-specific. Treatment of T cell clones with RTLs did not induce proliferation when added in solution, immobilized onto plastic microtiter plates, nor in combination with the addition of anti-CD28.

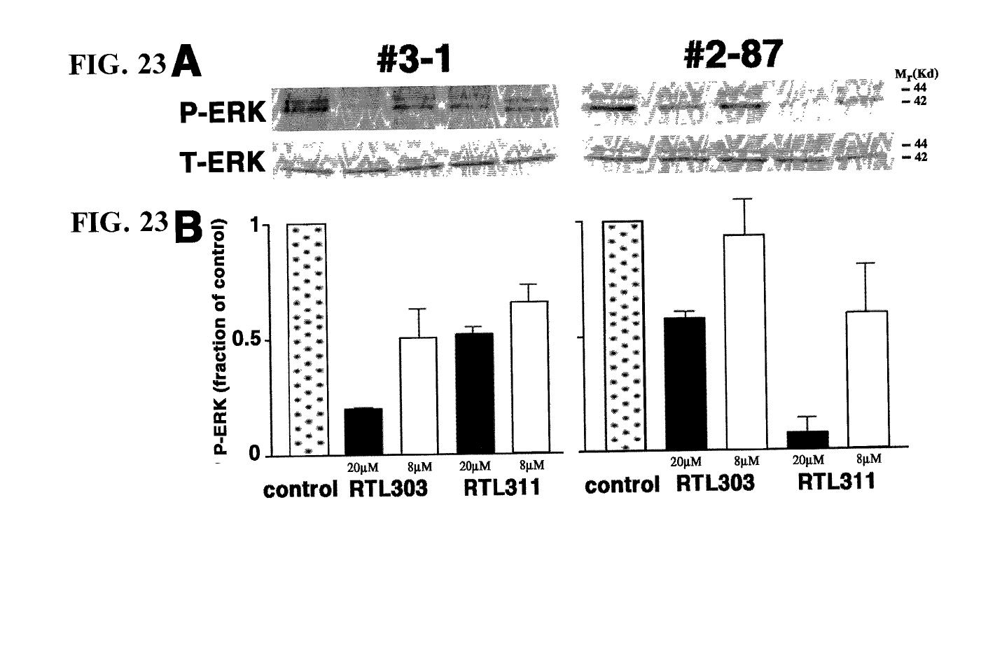

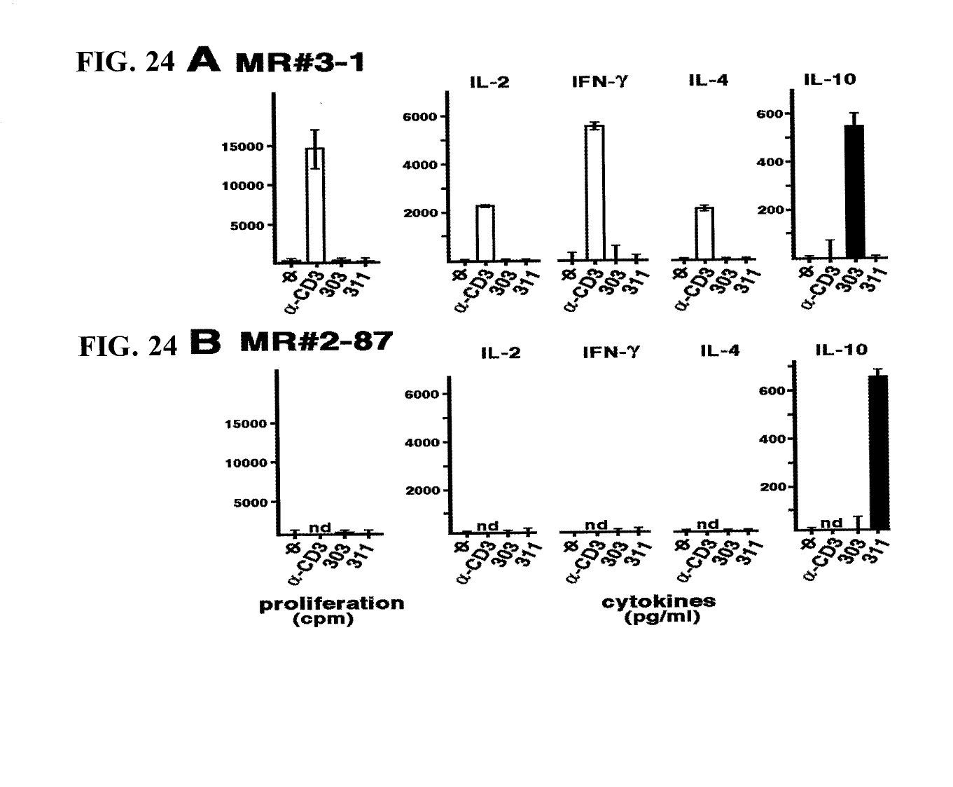

Upon activation with APC plus Ag, clone MR#3-1 (MBβ85-99 specific) and MR#2-87 (CABL specific) showed classic Th1 cytokine profiles that included IL-2 production, high IFN-γ and little or no detectable IL-4 or IL-10. As is shown in FIG. 24A, activation through the CD3-chain with anti-CD3 antibody induced an initial burst of strong proliferation and production of IL-2, IFN-7, and surprisingly, IL-4, but no IL-10. In contrast, upon treatment with RTL303, clone MR#3-1 continued production of IFN-y, but in addition dramatically increased its production of IL-10 (FIG. 24A). IL-10 appeared within 24 hours after addition of RTL303 and its production continued for more than 72 hours, to three orders of magnitude above the untreated or RTL311 treated control. In contrast, IL-2 and IL-4 levels did not show RTL induced changes (FIG. 24A). Similarly, after treatment with RTL311, Clone MR#2-87 (CABL specific) also showed a dramatic increase in production of IL-10 within 24 hours that continued for greater than 72 hours above the untreated or RTL303 treated control (FIG. 24B). Again, IL-2 and IL-4 levels did not show detectable RTL induced changes, and IFN-γ production remained relatively constant (FIG. 6B). The switch to IL-10 production was exquisitely Ag-specific, with the clones responding only to the cognate RTL carrying peptide antigen for which the clones were specific. The DR7 homozygous T cell clone CP#1-15 specific for MBP-85-99 showed no response to DR2-derived RTLs, indicating that RTL induction of IL-10 was also MHC restricted.

To assess the effects of RTL pre-treatment on subsequent response to antigen, T cell clones pretreated with anti-CD3 or RTLs were restimulated with APC/peptide, and cell surface markers, proliferation and cytokine production were monitored. RTL pre-treatment had no effect on the cell surface expression levels of CD25, CD69 or CD134 (OX40) induced by restimulation with APC/peptide compared to T cells stimulated with APC/peptide that had never seen RTLs, and there were no apoptotic changes observed over a 72 hour period using Annexin V staining (data not shown).

As anticipated, anti-CD3 pretreated T cells were strongly inhibited, exhibiting a 71% decrease in proliferation and >95% inhibition of cytokine production, with continued IL-2R (CD25) expression (Table 6; FIG. 25), a pattern consistent with classical anergy (Elder et al., 1994).

TABLE 6
Ag-specific inhibition of T cell clones by pre-culturing with RTLs.

| | Pre-Cultured with RTL303* | | Pre-Cultured with RTL311 | |
|---|---|---|---|---|
| Untreated | 20 μM | 10 μM | 20 μM | 10 μM |

Donor 1
Clone #3-1

| | | | | | |
|---|---|---|---|---|---|
| +APC** | 439 ± 221 | 549 ± 70 | 406 ± 72 | 491 ± 50 | 531 ± 124 |
| +APC + MBP-85-99 | 31725 ± 592 | 18608 ± 127 | 29945 ± 98 | 35172 ± 41 | 32378 ± 505 |
| (10 μg/ml) | | | | | |
| Inhibition (%) | — | −42.3 (p < 0.01) | −5.6 | 0 | 0 |

Clone #2-87

| | | | | | |
|---|---|---|---|---|---|
| +APC | 1166 ± 24 | 554 ± 188 | 1229 ± 210 | 1464 ± 281 | 1556 ± 196 |
| +APC + C-ABL- | 11269 ± 146 | 11005 ± 204 | 14298 ± 1669 | 5800 ± 174 | 7927 ± 575 |
| b2a3 (10 μg/ml) | | | | | |
| Inhibition (%) | — | 0 | 0 | −57.0 (p < 0.001) | −36.9 (p < 0.01) |
| Donor 2 | | | | | |

Clone #1-15

| | | | | | |
|---|---|---|---|---|---|
| +APC | 258 ± 48 | 124 ± 7 | ND | 328 ± 56 | ND |
| +APC + MBP-85-99 | 7840 ± 1258 | 7299 ± 1074 | ND | 8095 ± 875 | ND |
| (10 μg/ml) | | | | | |
| Inhibition (%) | — | −5.1 | | 0 | |

*Soluble RTL303 or RTL311 were co-cultured with T cell clones at 200,000 T cells/200 μl medium for 48 hours followed by washing twice with RPMI 1640 prior to the assay.
**2 × 10⁵ irradiated (2500 rad) autologous PBMC were added at ratio 4:1 (APC:T) for 3 days with ³H-Thymidine incorporation for the last 18 hr. The p values were based on comparison to “untreated” control.

Clone MR#3-1 showed a 42% inhibition of proliferation when pretreated with 20 μM RTL303, and clone MR#2-87 showed a 57% inhibition of proliferation when pretreated with 20 μM RTL311 (Table 6; FIG. 25). Inhibition of proliferation was also MHC class II-specific, as clone CP#1-15 (HLA-DR7 homozygous donor; MBβ85-99 specific) showed little change in proliferation after pre-treatment with RTL303 or RTL311 (Table I). Clone MR#3-1 pretreated with RTL303 followed by restimulation with APC/Ag showed a 25% reduction in IL-2, a 23% reduction in IFN-y and no significant changes in IL-4 production (FIG. 25). Similarly, clone MR#2-87 showed a 33% reduction in IL-2, a 62% reduction in IFN-γ production, and no significant change in IL-4 production. Of critical importance, however, both RTL-pretreated T cell clones continued to produce IL-10 upon restimulation with APC/peptide (FIG. 25).

The results presented above demonstrate clearly that the rudimentary TCR ligand embodied in the RTLs delivered signals to Th1 cells and support the hypothesis of specific engagement of RTLs with the (xp-TCR signaling. Signals delivered by RTLs have very different physiological consequences than those that occur following anti-CD3 antibody treatment.

In the system described herein, anti-CD3 induced strong initial proliferation and secretion of IL-2, IFN-γ, and IL-4 (FIG. 24). Anti-CD3 pre-treated T cells that were restimulated with APC/antigen had markedly reduced levels of proliferation and cytokine secretion, including IL-2, but retained expression of IL-2R, thus recapitulating the classical anergy pathway (FIG. 25). In contrast, direct treatment with RTLs did not induce proliferation, Th1 cytokine responses, or IL-2R expression, but did strongly induce IL-10 secretion (FIG. 24). RTL pretreatment partially reduced proliferation responses and Th1 cytokine secretion, but did not inhibit IL-2R expression upon restimulation of the T cells with APC/antigen. Importantly, these T cells continued to secrete IL-10 (FIG. 25). Thus, it is apparent that the focused activation of T cells through antibody crosslinking of the CD3-chain had vastly different consequences than activation by RTLs presumably through the exposed TCR surface. It is probable that interaction of the TCR with MHC/antigen involves more elements and a more complex set of signals than activation by crosslinking CD3-chains, and the results described herein indicate that signal transduction induced by anti-CD3 antibody may not accurately portray ligand-induced activation through the TCR. Thus, CD3 activation alone likely does not comprise a normal physiological pathway.

The signal transduction cascade downstream from the TCR is very complex. Unlike receptor tyrosine kinases, the cytoplasmic portion of the TCR lacks intrinsic catalytic activity. Instead, the induction of tyrosine phosphorylation following engagement of the TCR requires the expression of non-receptor kinases. Both the Src (Lck and Fyn) family and the Syk/ZAP-70 family of tyrosine kinases are required for normal TCR signal transduction (Elder et al., 1994). The transmembrane CD4 co-receptor interacts with the MHC class II beta-2 domain. This domain has been engineered out of the RTLs. The cytoplasmic domain of CD4 interacts strongly with the cytoplasmic tyrosine kinase Lck, which enables the CD4 molecule to participate in signal transduction. Lck contains an SH3 domain which is able to imediate protein-protein interactions (Ren et al., 1993) and which has been proposed to stabilize the formation of Lck homodimers, potentiating TCR signaling following co-ligation of the TCR and co-receptor CD4 (Eck et al., 1994). Previous work indicated that deletion of the Lck SH3 domain interfered with the ability of an oncogenic form of Lck to enhance IL-2 production, supporting a role for Lck in regulating cytokine gene transcription (Van Oers et al., 1996; Karnitz et al., 1992). T cells lacking functional Lck fail to induce Zap-70 recruitment and activation, which has been implicated in down-stream signaling events involving the MAP kinases ERK1 and ERK2 (Mege et al., 1996).

While the complete molecular signal transduction circuitry remains undefined, RTLs induce rapid antagonistic effects on 4-chain and ERK kinase activation. The intensity of the p21 and p23 forms of 4 increased together in a non peptide-Ag specific fashion (FIG. 21A), while the ratio of p23 to p21 varied in a peptide-Ag specific manner (FIG. 21B), due to a biased decrease in the level of the p23 moiety. The antagonistic effect on ERK phosphorylation also varied in a peptide-Ag specific manner (FIG. 21A). RTL treatment also induced marked calcium mobilization (FIG. 22). The fact that all three of these pathways were affected in an antigen specific fashion strongly implies that the RTLs are causing these effects through direct interaction with the TCR.

The results described herein demonstrate the antigen-specific induction by RTLs of IL-10 secretion. This result was unexpected, given the lack of IL-10 production by the Th1 clones when stimulated by APC/antigen or by anti-CD3 antibody. Moreover, the continued secretion of IL-10 upon restimulation of the RTL pre-treated clones with APC/antigen indicates that this pathway was not substantially attenuated during reactivation. This result suggests that TCR interaction with the RTL results in default IL-10 production that persists even upon re-exposure to specific antigen. The elevated level of IL-10 induced in Th1 cells by RTLs has important regulatory implications for autoimmune diseases such as multiple sclerosis because of the known anti-inflammatory effects of this cytokine on Th1 cell and macrophage activation (Negulescu et al., 1996).

It is likely that the pathogenesis of MS involves autoreactive Th1 cells directed at one or more immunodominant myelin peptides, including MBP-85-99. Without being bound by theory, RTLs such as RTL303 could induce IL-10 production by these T cells, thus neutralizing their pathogenic potential. Moreover, local production of IL-10 after Ag-stimulation in the CNS could result in the inhibition of activation of bystander T cells that may be of the same or different Ag specificity, as well as macrophages that participate in demyelination. Thus, this important new finding implies a regulatory potential that extends beyond the RTL-ligated neuroantigen specific T cell. RTL induction of IL-10 in specific T cell populations that recognize CNS antigens could potentially be used to regulate the immune system while preserving the T cell repertoire, and may represent a novel strategy for therapeutic intervention of complex T cell mediated autoimmune diseases such as MS.

### Example 13

### Vaccination Induced Bystander Suppression for the Treatment of Autoimmune Disease

The pathogenesis of a variety of human diseases including multiple sclerosis (MS), rheumatoid arthritis, diabetes, autoimmune uveitis, transplant rejection, chronic beryllium disease and graft-vs-host disease appear to involve antigen-specific CD4+ T cells. It is thought that pathogenic T cells home to the target tissue where autoantigen is present, and, after local activation, selectively produce Th1 lymphokines. This cascade of events leads to the recruitment and activation of lymphocytes and monocytes that ultimately destroy the target tissue. Activation of CD4+ T cells in vivo is a multi-step process initiated by co-ligation of the TCR and CD4 by the MHC class II/peptide complex present on APC (signal 1), as well as co-stimulation through additional T cell surface molecules such as CD28 (signal 2). Ligation of the TCR in the absence of co-stimulatory signals has been shown to disrupt normal T cell activation, inducing a range of responses from anergy to apoptosis. Within the context of this model of T cell activation, a direct approach toward Ag-driven immunosuppression would be to present the complete TCR ligand, Ag in the context of MHC, in the absence of costimulatory signals that are normally provided by specialized APCs.

Bystander suppression is the effect produced by regulatory cells, in most cases T cells, responding to antigen expressed by a particular tissue that is proximal to autoantigens. The regulatory cells then produce a microenvironment, most likely through the production of cytokines (e.g.TGF-β, IL-10 or IL-13) which suppress the response of the autoimmune cells. The ability to induce bystander T regulatory cells by vaccination has promising potential for an immune based autoimmune therapy, as the difficult task of determining disease specific autoantigens is no longer necessary. Vaccines strategies designed to induce these antigen-specific regulatory cells only need to express antigens specific to the tissue undergoing autoimmune attack. Therefore, in diseases where the autoantigen is unknown or where there may be multiple antigens (for example, multiple sclerosis (MS), type 1 diabetes, or rheumatoid arthritis) vaccination only needs to be directed to antigens particular to those tissues in conjunction with MHC. Thus, for MS, vaccination is, for example, directed to myelin basic protein (see above), for diabetes, vaccination is, for example, directed to insulin, and for rheumatoid arthritis, vaccination is, for example, directed to Type II collagen respectively.

TABLE 7
Examples of Human Autoimmune Disorders

| Human Disease | Animal Model | Antigen of Use |
|---|---|---|
| Multiple Sclerosis | experimental autoimmne | Myelin basic protein |
| | encephalitis (EAE) | (MBP) proteolipid |
| | mouse model and Lewis | protein (PLP) and |
| | rat | myelin oligodedrocyte |
| | | glycoprotein |
| Diabetes | NOD mice | Insulin, glutamate |
| | | decarboxylase |
| Arthritis and related | Chicken, Mice and Rats | Type II collagen |
| MCTD (mixed con- | | |
| nective tissue disease) | | |
| Hashimoto's | Mice, Lewis Rats, and | Thyroglobulin, |
| Thyroiditis, | OS chickens | Thyrodoxin |
| Grave's Disease | | |
| Uveitus | Mice | S-antigen |
| Inflammatory Bowel | MDrla Knockout Mice | Ach (acetylcholine) |
| Disease | | Receptor |
| Polyarteritis | Mice | HepB Antigen |
| Myasthenia Gravis | mice | |
| Transplantation | Mice | Insulin, glutamate |
| rejection | Islet cell transplantation | decarboxylase |

There are several animal based autoimmune models that can be used to test the use of MHC/peptide complex for the treatment of an autoimmune disorder. Table 7 lists several exemplary immune mediated disorders that can be treated using a peptide/MHC complex. For example, the non-obese diabetic (NOD) mouse model is an animal model system wherein animals develop diabetes with increasing age. To test the efficacy of a particular antigen/MHC complex, groups of animals at the prediabetic stage (4 weeks or younger) are vaccinated with, for example, insulin-MHC complex. The number of animals developing diabetes, and the rate that the animals develop diabetes, is then analyzed. Similarly, in the Hashimoto's mouse model system, to test the efficacy of a vaccine, groups of animals prior to the development of symptoms are vaccinated with a thyrodoxin/MHC complex. The number of animals developing the disease, and the rate that the animals develop the disease, is then analyzed.

In the NOD model or in the Hashimoto's model, the antigen/MHC complex delays the progression of the disease, or provides protection from developing the disease, when compared to animals primed with a nucleic acid encoding an unrelated antigen or as compared to untreated controls. The immune cell type that provides this protection is then studied by adoptive transfer studies to untreated mice (e.g in NOD mice the transplantation of specific populations of immune cells, such as CD4, CD8, NK or B cells, into untreated NOD animals). Thus the cell population responsible for the regulation of the inflammatory response is determined.

For the adoptive transfer experiments, groups of Balb/c are given either peptide/MHC complex or a nucleic acid encoding the peptide/MHC complex. CD4+, CD8+, B220 and NK1.1+ cells are isolated by immunomagnetic bead separation. These different cell types are then transferred to naive NOD mice by IV injection. These animals receiving the transferred cells are then observed form signs of disease onset. Animals receiving peptide/MHC complex exhibit a delayed onset or no disease progression compared to controls.

The above Examples illustrate the efficacy of the two-domain MHC molecules. While the experimental details concern the MHC class II β1α1 polypeptides, it will be appreciated that these data fully support application of MHC class I α1α2 polypeptides.

In view of the many possible embodiments to which the principles of our invention may be applied, it should be recognized that the illustrated embodiment is only a preferred example of the invention and should not be taken as a limitation on the scope of the invention. Rather, the scope of the invention is defined by the following claims. We therefore claim as our invention all that comes within the scope and spirit of these claims.

### REFERENCES

Abbas, A. K., Murphy, K. M. 1996. Functional diversity of helper T lymphocytes. Nature 383:787.

Altman, J. D. et al. Phenotypic analysis of antigen-specific T lymphocytes. Science 274, 94-96 (1996).

Arimilli, S., Cardoso, C., Mukku, P., Baichwal, V. & Nag, B. Refolding and reconstitution of functionally active complexes of human leukocyte antigen DR2 and myelin basic protein peptide from recombinant alpha and beta polypeptide chains. Journal of Biological Chemistry 270(2), 971-977 (1995).

Auffray et al. (1984). Isotypic and allotypic variation of human class II histocompatibility antigen alpha-chain genes. Nature 308 (5957), 327-333.

Ausubel et al. (1987). In Current Protocols in Molecular Biology, Greene Publishing Associates and Wiley-Intersciences.

Bebo, B. F. Jr., Jeanette C. Schuster, Arthur A. Vandenbark, Halina Offner. 1999. Androgens alter the cytokine profile and reduce encephalitogenicity of myelin reactive T cells. J. Immunol. 162:35.

Benoist et al. (1983). The murine Ia alpha chains, E alpha and A alpha, show a surprising degree of sequence homology. Proc. Natl. Acad. Sci. U.S.A. 80 (2), 534-538.

Boniface, J. J. & Davis, M. M. T-cell recognition of antigen. A process controlled by transient intermolecular interactions. Annals New York Acad. Sciences. 766, 62-69 (1995).

Bourdette, D. N. et al. Myelin basic protein specific T cells in the CNS and lymph nodes of rats with EAE are different. J. Neurosci. Res. 30, 308-315 (1991).

Brogdon, J., Eckels, D. D., Davies, C., White, S. and Doyle, C. 1998. A site for CD4 binding in the beta-I domain of the MHC class II protein HLA-DR1. J. Immunol. 161:5472.

Brown, J. H., Jardetzky, T. S., Gorga, J. C., Stern, L. J., Urban, R. G. & Strominger, J. L Three dimensional structure of the human class II histocompatibility antigen HLA-DRI. Nature 364, 33-39 (1993).

Browning et al., (1995). The HLA-A, B,C genotype of the class I negative cell line Daudi reveals novel HLA-A and -B alleles. Tissue Antigens 45 (3), 177-187.

Burley, S. K., and Petsko, G. A. (1985) Science 229, 23-28.

Burrows, G. G. et al. Variation in H-2K^{k} peptide motif revealed by sequencing naturally processed peptides from T cell hybridoma class I molecules. J. Neurosci. Res. 45, 803-811 (1996).

Burrows, G. G. et al. Multiple Class I Motifs Revealed by Sequencing Naturally Processed Peptides Eluted from Rat T Cell MHC Molecules. Rapid Communication. J Neurosci. Res. 49, 107-116 (1997).

Burrows, G. G. Bebo, B. F. Jr., Adlard, K. L., Vandenbark, A. A., and Offner, H. (1998) J. Immunol. 161, 5987-5996

Burrows, G. G., Chang, J. W., Bachinger, H-P., Bourdette, D. N., Wegmann, K. W., Offner, H. and Vandenbark A. A. (1999) Protein Engineering 12, 771-778

Burrows, G. G., Adlard, K. L., Bebo, B. F. Jr., Chang, J. W., Tenditnyy, K.,Vandenbark, A. A. and Offner, H. (2000) J. Immunol. 164, 6366-6371

Cammarota, G. et al. Identification of a CD4 binding site on the b₂ domain of HLA-DR molecules. Nature 356, 799-801 (1992).

Caspi, R. R. et al. A new model of autoimmune disease: Experimental autoimmune uveorentinitis induced in mice with two different retinal antigens. J. Immunol. 140, 1490-1495 (1988).

Chaurhary et al. (1989). Nature 339: 394-397.

Chen, G. C. and Yang, J. T. (I1977) Anal. Letters 10, II195-1207.

Cobbold, S. P., Nash, J. A., Prospero, T. D. & Waldham, H. Therapy with monoclonal antibodies by elimination of T-cell subsets in vivo. Nature 312, 548-551 (1988).

Cobbold, S. P., Nash, J. A., Prospero, T. D. & Waldham, H. Therapy with monoclonal antibodies by elimination of T-cell subsets in vivo. Nature 312, 548-551 (1988).

Collins et al. Nature 371 (6498): 626-629 (1994).

Compton, L. A., and Johnson, W. C. Jr. (I1986) Analytical Biochemistry 155, 155-67

Connolly, M. L. (I1983) Science 221, 709-13

Connolly, M. L. (I1986) Biopolymers 25, 1229-47.

Corr, M. et al. T cell receptor-MHC class I peptide interactions: affinity, kinetics, and specificity. Science 265, 946-949 (1995).

Cush, J. J. & Lipsky, P. E. Phenoytpic analysis of synovial tissue and peripheral blood lymphocytes isolated from patients with rheumatoid arthritis. Arthritis Rheum. 31, 1230-1238 (1988).

Das et al. (1983). Structure and nucleotide sequence of the heavy chain gene of HLA-DR. proc. Natl. Acad. Sci. U.S.A. 80 (12), 3543-3547.

Davis, M. M., Boniface, J. J., Reich, Z., Lyons, D., Hampl, J., Arden, B., Chien, Y. 1998.

Ligand recognition by alpha beta T cell receptors. Annu. Rev. Immunol. 16:523.

Derman, A. I., Prinz, W. A., Belin, D. & Beckwith, J. Mutations that allow disulfide bond formation in the cytoplasm of Escherichia coli. Science 262, 1744-1747 (1993).

Desbarats, J., Freed, J. H., Campbell, P. A., & Newell, M. K. Fas (CD95) expression and death-mediating function are induced by CD4 cross-linking on CD4+ T cells. PNAS 93, 11014-11018 (1996).

Eck, M. J., Atwell, S. K., Shoelson, S. E., and Harrison, S. C. 1994. Structure of the regulatory domains of the Src-family tyrosine kinase Lck. Nature 368:764.

Edwards et al. Science 276 (5320): 1868-1871 (1997).

Elder, M. E., Lin, D., Clever, J., Chan, A. C., Hope, T. J., Weiss, A., and Parslow, T. G. 1994. Human severe combined immunodeficiency due to a defect in ZAP-70, a T cell tyrosine kinase. Science 264:1596.

Estess et al. (1986). Sequence analysis and structure-function correlations of murine q, k, u, s, and f haplotype I-A beta CDNA clones. Proc. Natl. Acad. Sci. U.S.A. 83 (11), 3594-3598.

Ferrin, T. E., Huang, C. C., Jarvis, L. E. & Langridge, R. The MIDAS display system. J. Mol. Graphics 6, 13-27 (1988).

Fleury et al., CELL 66, 1037-1049 1991.

Fearon, D. T., Locksley, R. M. 1996. The instructive role of innate immunity in the acquired immune response. Science 272:50.

Fremont, et al. Structures of an MHC class II molecule with covalently bound single peptides. Science 272, 1001-1004 (1996).

Germain, R. N. 1994. Major histocompatibility complex-dependent antigen processing and peptide presentation: providing ligands for the clonal activation of T lymphocytes. Cell 76:287.

Gold, D. P., H. Offner, D. Sun, S. Wiley, A. A. Vandenbark and D. B. Wilson. 1991. Analysis of T cell receptor chains in Lewis rats with experimental autoimmune encephalomyelitis: Conserved complementarity determining region 3. J. Exp. Med. 174:1467.

Golding, H., J. McCluskey, T. I. Munitz, R. N. Germain, D. H. Margulies and A. Singer. 1985. T-cell recognition of a chimaeric class II/class I MHC molecule and the role of L3T4. Nature, 317:425.

Govaerts, A. et aL HLA and multiple sclerosis: population and family studies. Tissue Antigens 25, 187-199 (1985).

Harlow and Lane (1988). Antibodies, A Laboratory Manual, Cold Spring Harbor Laboratory, New York.

Hashim, G. A., Day, E. D., Fredane, L., Intintola, P., and Carvalho, E. Biological activity of region 65 to 102 of the myelin basic protein. J. Neurosci. Res. 16, 467-478 (1986).

Heiden, W., Moeckel, G., and Brickmann, J. (1993) J. Comp.-Aided Mol. Design 7, 503-514.

Hemmer, B., Fleckenstein, B. T., Vergelli, M., Jung, G., McFarland, H., Martin, R. and Wiesmuiller, K. H. (1997) J. Exp. Med. 185, 1651-1660.

Housset, D., Habersetzer-Rochat, C., Astier, J. P. & Fontecilla-Camps, J. C. Crystal structure of toxin II from the scorpion Androctonus Australis Hector refined at 1.3 angstroms resolution. J. Mol. Biol. 238, 88 (1994).

Howell, M. D. et al. Vaccination against experimental allergic encephalomyelitis with T-cell receptor peptides. Science 246, 668-670 (1989).

Huang, B., Yachou, A., Fleury, S., Hendrickson, W. & Sekaly, R. Analysis of the contact sites on the CD4 molecule with class II MHC molecule. J. Immunol. 158, 216-225 (1997).

Innis et al. (1990). PCR Protocols, A Guide to Methods and Applications, Innis et al. (eds.), Academic Press, Inc., San Diego, Calif.

Jameson, B. A., McDonnel, J. M., Marini, J. C. & Kormgold, R. A rationally designed CD4 analogue inhibits experimental allergic encephalomyelitis. Nature 368, 744-746 (1994).

Janes, P. W., Ley, S. C., Magee, A. I. 1999. Aggregation of lipid rafts accompanies signaling via the T cell antigen receptor. J. Cell Biol. 14: 447.

Janeway, C. A. Jr. and Bottomly, K. (1994) Cell 76, 275-285.

Janeway & Travers (1997). Immunobiology: the immune system in health and disease, Current Biology Ltd./Garland Publishing, Inc. New York.

Janeway, C. A., Jr. 1998. Presidential Address to The American Association of Immunologists. The road less traveled by: the role of innate immunity in the adaptive immune response. J. Immunol. 161:539.

Kamitz, L., Sutor, S. L., Torigoe, T., Reed, J. C., Bell, M. P., McKean, D. J., Leibson, P. J., and Abraham, R. T. 1992. Effects of p561ck deficiency on the growth and cytolytic effector function of an interleukin-2-dependent cytotoxic T-cell line. Mol. Cell. Biol. 12:4521.

Kato et al., (1993). Molecular analysis of HLA-B39 subtypes. Immunogenetics 37 (3), 212-216.

Kelly & Trowsdale. (1985). Complete nucleotide sequence of a functional HLA-DP beta gene and the region between the DP beta 1 and DP alpha 1 genes: comparison of the 5′ ends of HLA class II genes Nucleic Acids Res. 13 (5), 1607-1621.

Kersh, E. N., Kersh, G. J., Allen, P. M. 1999. Partially phosphorylated T cell receptor zeta molecules can inhibit T cell activation. J. Exp. Med. 190:1627.

King, J. and Leimmli, U.K. Bacteriophage T4 tail assembly: structural proteins and their genetic identification. J. Mol. Biol. 75, 315-337 (1973).

Konig, R., Shen, X. & Germain, R.N. Involvement of both major histocompatibility complex class II and B chains in CD4 function indicates a role for ordered oligomerization in T cell activation. J. Exp. Med. 182, 779-787 (1995).

König, R., Huang, L. Y. & Germain, R. MHC class II interaction with CD4 mediated by a region analogous to the MHC class I binding site for CD8. Nature 356, 796-798 (1992).

Kozono, H., White, J., Clements, J., Marrack, P. & Kappler, J. Production of soluble MHC class II proteins with covalently bound single peptides. Nature 369, 151-154 (1994).

Kress et al., (1983). Alternative RNA splicing in expression of the H-2K gene. Nature 306 (5943), 602-604.

Kyle, V., Coughlan, R. J., Tighe, H., Waldmann, H., and Hazleman, B. L. 1989. Beneficial effect of monoclonal antibody to interleukin 2 receptor on activated T cells in rheumatoid arthritis. Ann Rheum Dis. 48:428.

Larhammar et al. (1983). Exon-intron organization and complete nucleotide sequence of a human major histocompatibility antigen DC beta gene. Proc. Natl. Acad. Sci. U.S.A. 80 (23), 7313-7317.

Lawrence et al. (1985). The genomic organisation and nucleotide sequence of the HLA-SB(DP) alpha gene Nucleic Acids Res. 13 (20), 7515-7528.

Li, W., Whaley, C. D., Mondino, A. and Mueller, D. L. 1996. Blocked Signal Transduction to the ERK and JNK Protein Kinases in Anergic CD4+ T Cells Science 271:1272.

Li, Y., Li, H., Martin, R., and Mariuzza, R. A. (2000) J. Mol. Biol. 304, 177-188.

MacDonald, H. R., Casanova, J. L., Maryanski, J. L., Cerottini, J. C. Oligoclonal expansion of major histocompatibility complex class I-restricted cytolytic T lymphocytes during a primary immune response in vivo: direct monitoring by flow cytometry and polymerase chain reaction. J. Exp. Med. 177, 1487-1492 (1993).

Madden, D. R., Gorga, J. C., Strominger, J. L. & Wiley, D. C. The structure of HLA-B27 reveals nonamer self-peptides bound in an extended conformation. Nature 353, 321-325 (1991).

Martenson, R. E. Myelin basic protein speciation. in Experimental Allergic Encephalomyelitis: A useful Model for Multiple Sclerosis. 1984. Alan R. Liss, Inc., 150 Fifth Avenue, New York, N.Y. 1001 1. pages 511-521.

Martin R, Utz U, Coligan J E, Richert J R, Flerlage M, Robinson E, Stone R, Biddison W E, McFarlin D E, McFarland H F. 1992. Diversity in fine specificity and T cell receptor usage of the human CD4+ cytotoxic T cell response specific for the immunodominant myelin basic protein peptide 87-106. J Immunol. 148:1359.

Matsui, K., Boniface, J. J., Steffner, P., Reay, P. A., Davis, M. M. Kinetics of T-cell receptor binding to peptide/I-E^{K} complexes: correlation of the dissociation rate with T-cell responsiveness. Proc. Natl. Acad. Sci. U.S.A. 91, 12862-12866 (1994).

Matsui, K. et al. Low affinity interaction of peptide-MHC complexes with T cell receptors. Science 254, 1788-1791 (1991).

McHeyzer, M. G., Davis, M. M. Antigen specific development of primary and memory T cells in vivo. Science 268, 106-111 (1995).

Mege, D., Di Bartolo, V., Germain, B., Tuosto, L., Michel, F., and Acuto, O. 1996. Mutation of Tyrosines 492/493 in the Kinase Domain of ZAP-70 Affects Multiple T-cell Receptor Signaling Pathways J. Biol. Chem. 271:32644.

Miltenyi et al. Cytometry 11: 231-238 (1990).

Mitragotri et al. Pharmaceutical Research 13 (3): 411-20 (1996).

Moebius, U., Pallai, P., Harrison, S.C. & Reinherz, E.L. Delineation of an extended surface contact area on human CD4 involved in class II MHC binding. PNAS 90, 8259-8263 (1993).

Mosmann, T. R., Cherwinski, H., Bond, M. W., Giedlin, M. A., Coffinan, R. L. 1986. Two types of murine helper T cell clones. I. Definition according to profiles of lymphokine activities and secreted proteins. J. Immunol. 136:2348.

Moore et al., (1982). DNA sequence of a gene encoding a BALF/c mouse Ld transplantation antigen. Science 215 (4533), 679-682.

Murthy, V. L., and Stern, L. J. (1997) Structure 5, 1385-1396.

Nag, B., Deshpande, S. V., Sharma, S. D., & Clark, B. R. Cloned T cells internalize peptide from bound complexes of peptide and purified class II major histocompatibility complex antigen. J. Biol. Chem. 268, 14360-14366 (1993).

Nag, B., Kendrick, T., Arimilli, S., Yu, S. C., & Sriram, S. Soluble MHC II-peptide complexes induce antigen-specific apoptosis in T cells. Cell. Immunol. 170, 25-33 (1996).

Nag, B., Passmore, D., Kendrick, T., Bhayani, H., & Sharma, S. D. N-linked oligosaccharides of murine major histocompatibility complex class II molecule. Role in antigenic peptide binding, T cell recognition, and clonal nonresponsiveness. J. Biol. Chem. 267, 22624-22629 (1992).

Nag B., Arimilli S., Mukku P. V. & Astafieva, I. Functionally active recombinant alpha and beta chain-peptide complexes of human major histocompatibility class II molecules. Journal of Biological Chemistry 271(17), 10413-10418 (1996).

Nag B. et al. Stimulation of T cells by antigenic peptide complexed with isolated chains of major histocompatibility complex class II molecules. Proceedings of the National Academy of Sciences of the United States of America 90(4), 1604-1608 (1993).

Negulescu, P. A., T. B. Krasieva, A. Khan, H. H. Kerschbaum, and M. D. Cahalan. 1996. Polarity of T cell shape, motility, and sensitivity to antigen. Immunity 4:421.

Nicolle, M. W. et al. Specific tolerance to an acetylcholine receptor epitope induced in vitro in myasthenia gravis CD4+ lymphocytes by soluble major histocompatibility complex class II-peptide complexes. J. Clin Invest. 93, 1361-1369 (1994).

Oksenberg, J. R. et al. Selection of T-cell receptor V-D-J gene rearrangements with specificity for a MBP peptide in brain lesions of MS. Nature 362, 68-70 (1993).

Olerup, O., and Zetterquist, H. 1992. HLA-DR typing by PCR amplification with sequence specific primers (PCR-SSP) in two hours: An alternative to serological DR typing in clinical practice including donor-recipient matching in cadaveric transplantation. Tissue Antigens 39:225.

Ota, K. et al. T cell recognition of an immunodominant myelin basic protein epitope in multiple sclerosis. Nature 346, 183-187 (1990).

Paterson, P. Y. (1973) J. Chron. Dis. 26, 119-26

Paterson, P. Y. Multiple sclerosis: An immunologic reassessment. J. Chron. Dis. 26, 119-125 (1981).

Ploegh, H. and Benaroch, P. (1993) Nature 364, 16-17.

Provencher, S. W. and Glockner, J. (1981) Biochemistry 20, 33-37.

Quill, H. & Schwartz, R. H. Stimulation of normal inducer T cell clones with antigen presented by purified Ia molecules in planer lipid membranes: specific induction of a long-lived state of proliferative nonresponsiveness. J. Immunol. 138, 3704-3712 (1987).

Redpath, S., Alam, S. M. Lin, C. M., O'Rourke, A. M., and Gascoigne, N. R. 1999. Cutting edge: Trimolecular interaction of TCR with MHC class II and bacterial superantigen shows a similar affinity to MHC:peptide ligands. J. Immunol. 163:6.

Reiner, S. L., Wang, Z. E., Hatam, F., Scott, P., Locksley, R. M. TH1 and TH2 cell antigen receptors in experimental leishmaniasis. Science 259, 1457-1460 (1993).

Ren, R., Mayer, B. J., Cicchetti, P., and Baltimore, D. 1993. Identification of a ten-amino acid proline-rich SH3 binding site. Science 259:1157.

Rhode, P. R. et al. Single-chain MHC class II molecules induce T cell activation and apoptosis. J. Immunol. 157, 4885-4891 (1996).

Sambrook et al. (1989). In Molecular Cloning: A Laboratory Manual, Cold Spring Harbor, N.Y.

Schaeffer, H. J. and Weber, M. J. 1999. Mitogen-activated protein kinases: specific messages from ubiquitous messengers. Mol. Cell. Biol. 19:2435.

Schafer, P. H., Pierce, S. K. and Jardetzky T. S. (1995) Seminars in Immunology 7, 389-98.

Schepart et al., (1986). The nucleotide sequence and comparative analysis of the H-2Dp class I H-2 gene. J. Immunol. 136 (9), 3489-3495.

Schwartz, R. H. Models of T cell anergy: is there a common molecular mechanism? J. Exp. Med. 184, 1-8 (1996)

Service et al. Science 277(5330): 1199-1200 (1997).

Sharma, S. D. et al. Antigen-specific therapy of experimental allergic encephalomyelitis by soluble class II major histocompatibility complex-peptide complexes. PNAS 88:11465-11469 (1991).

Sloan-Lancaster, J., Shaw, A. S., Rothbard, J. B., Allen, P. M. 1994. Partial T cell signaling: Altered phospho-zeta and lack of Zap70 recruitment in APL-induced T cell anergy. Cell 79:913.

Smith, K. J., Pyrdol, J., Gauthier, L., Wiley, D. C., and Wucherpfennig, K. W. (1998) J. Exp .Med. 188, 1511-1520

Spack, E. G. et al. Induction of tolerance in experimental autoimmune myasthenia gravis with solubilized MHC class II:acetylcholine receptor complexes. J. Autoimmun. 8, 787-807 (1995).

Steinle et al., (1992). Isolation and characterization of a genomic HLA-Cw6 clone. Tissue Antigens 39(3), 134-137.

Steinman, L. Autoimmune disease. Sci. Am. 269, 106-114 (1993).

Studier, F. W., A. H. Rosenberg, J. J. Dunn, and J. W. Dubendorff. 1990. Use of T7 RNA polymerase to direct expression of cloned genes. Methods Enzymol. 185:60.

Swanborg, R. H. Autoimmune effector cells. V. A monoclonal antibody specific for rat helper T lymphocytes inhibits adoptive transfer of auto-immune encephalomyelitis. J Immunol. 130, 1503-1505 (1983).

Syha, J., Henkes, W. & Reske, K. Complete cDNA sequence coding for the MHC class II RTI.B -chain of the Lewis rat. Nuc. Acids. Res. 17(10), 3985 (1989).

Syha-Jedelhauser, J., Wendling, U. & Reske, K. Complete coding nucleotide sequence of cDNA for the Class II RTI.B 13-chain of the Lewis rat. Biochim. Biophys. Acta 1089, 414-416 (1991).

Sykulev, Y. et al. Kinetics and affinity of reactions between an antigen-specific T cell receptor and peptide-MHC complexes. Immunity 1, 15-22 (1994).

ten Bosch, G. J., Toornvliet, A. C., Friede, T., Melief, C. J., and Leeksma, O. C. 1995. Recognition of peptides corresponding to the joining region of p21 OBCR-ABL protein by human T cells. Leukemia 9:1344.

Thompson, D. and Larson, G. Western blots using stained protein gels. Biotechniques 12, 656-658 (1992).

Tonnell et al. (1985). Do beta: a new beta chain gene in HLA-D with a distinct regulation of expression. EMBO J. 4 (11), 2839-2847.

Vandenbark, A. A., Vainiene, M., Celnik, B., Hashim, G. A., Buenafe, A. C. & Offner, H. Definition of encephalitogenic and immunodominant epitopes of guinea pig myelin basic protein (Gp-BP) in Lewis rats tolerized neonatally with Gp-BP peptides. J. Immunol. 153, 852-861 (1994).

Vandenbark, A. A., Hashim, G. & Offner, H. Immunization with a synthetic T-cell receptor V-region peptide protects against experimental autoimmune encephalomyelitis. Nature 341, 541-544 (1989).

Vandenbark, A. A., Gill, T. & Offner, H. A myelin basic protein specific T lymphocyte line which mediates EAE. J. Immunol. 135, 223-228 (1985).

Van Oers, N., Killeen, N., and Weiss, A. 1996. Lck regulates the tyrosine phosphorylation of the T cell receptor subunits and ZAP-70 in murine thymocytes J. Exp. Med. 183:1053.

Veillette, A., Bookman, M. A., Horak, E. M. & Bolen, J. B. The CD4 and CD8 T cell surface antigens are associated with the internal membrane tyrosine-protein kinase p₅₆^{lck}. Cell 55, 301-308 (1988).

Vilanova, M., Tavares, D., Ferreira, P., Oliveira, L., Nobrega, A., Appelberg, R., and Arala-Chaves M. 1996. Role of monocytes in the up-regulation of the early activation marker CD69 on B and T murine lymphocytes induced by microbial mitogens. Scand J Immunol. 43:155.

Walker, P. R., Ohteki, T., Lopez, J. A., MacDonald, H. R., Maryanski, J. L. Distinct phenotypes of antigen-selected CD8 T cells emerge at different stages of an in vivo immune response. J. Immunol. 155, 3443-3452 (1995).

Walter et al., (1994). Sequence, expression, and mapping of rat Mhc class Ib gene. Immunogenetics 39 (5), 351-354.

Walter et al., (1995). Genomic organization and sequence of the rat major histocompatibility complex class Ia gene RT1.Au Immunogenetics 41 (5), 332.

Wegmann K W, Zhao W., Griffin A C.,and Hickey W F. Identificationofmyocarditogenic peptides derived from cardiac myosin capable of inducing experimental allergic myocarditis in the Lewis rat. The utility of a class II binding motif in selecting self-reactive peptides. J. Immunol. 153(2), 892-900 (1994).

Weinberg, A. D. et al. TGF-β enhances the in vivo effector function and memory phenotype of Ag-specific T helper cells in EAE. J. Immunol. 148, 2109-2117 (1992).

Weinberg, A. D. et al. Target organ specific upregulation of the MRC OX-40 marker and selective production of Th1 lymphokine mRNA by encephalitogenic T helper cells isolated from the spinal cord of rats with experimental autoimmune encephalomyelitis. J. Immunol. 152, 4712-5721 (1994).

Weinberg, A. D., Bourdette, D. N., Sullivan, T. J., Lemon, M., Wallin, J. J., Maziarz, R., Davey, M., Palida, F., Godfrey, W., Engleman, E., Fulton, R. J., Offner, H., and Vandenbark, A. A. 1996. Selective depletion of myelin-reactive T cells with the anti-OX-40 antibody ameliorates autoimmune encephalomyelitis. Nature Medicine 2:183.

Weiner, H. L. et al. Double-blind pilot trial of oral tolerization with myelin antigens in MS. Science 259, 1321-1324 (1993).

White, J., M. Blackman, J. Bill, J. Kappler, P. Marrack, D. P. Gold and W. Born. 1989. Two better cell lines for making hybridomas expressing specific T cell receptors. J. Immunol. 143:1822.

Wutlfing, C., Rabinowitz, J. D., Beeson, C., Sjaastad, M. D., McConnell, H. M. and Davis, M. M. 1997. Kinetics and Extent of T Cell Activation as Measured with the Calcium Signal. J. Exp. Med. 185:1815.

Yednock, T. A. et al. Prevention of experimental autoimmune encephalomyelitis by antibodies against 4/β1 integrin. Nature 356, 63 (1992).

Zhao, B., Carson, M., Ealick, S. E. & Bugg, C. E. Structure of scorpion toxin variant-3 at 1.2 angstroms resolution. J. Mol. Biol. 227, 239-252 (1992).

Zinn-Justin, S., Guenneugues, M., Drakopoulou, Gilquin, B., Vita, C. & Menez, A. Transfer of a beta-hairpin from the functional site of snake curaremimetic toxins to the alpha/beta scaffold of scorpion toxins: Three-dimensional solution structure of the chimeric protein. Biochemistry 35(26): 8535-43 (1996).

1. A purified MHC Class II polypeptide comprising covalently linked first and second domains, wherein:
the first domain is a human MHC class II β1 domain and the second domain is a mammalian MHC class II α1 domain and wherein the amino terminus of the second domain is covalently linked to the carboxy terminus of the first domain and wherein the MHC class II molecule does not include an α2 or a β2 domain; or
the first domain is a human MHC class I α1 domain and the second domain is a mammalian MHC class I α2 domain, and wherein the amino terminus of the second domain is covalently linked to the carboxy terminus of the first domain and wherein the MHC class I molecule does not include an α3 domain.

2. The polypeptide of claim 1 wherein the covalent linkage between the first and second domains is provided by a peptide linker sequence.

3. The polypeptide of claim 1 wherein the polypeptide further comprises, covalently linked to the amino terminus of the first domain, a third domain comprising an antigenic determinant.

4. The polypeptide of claim 3, wherein the antigenic determinant is a peptide antigen.

5. The polypeptide of claim 4, wherein the covalent linkage between the first and third domains is provided by a peptide linker sequence.

6. The polypeptide of claim 1, further comprising an antigenic determinant associated with the polypeptide by non-covalent interaction.

7. The polypeptide of claim 6, wherein the antigenic determinant is a peptide antigen.

8. The polypeptide of claim 1 wherein the polypeptide further comprises a covalently linked detectable marker or toxic moiety.

9. The polypeptide of claim 1, wherein the covalent linkage between the β1 and α1 domains is provided by a peptide linker sequence.

10. A nucleic acid molecule encoding the polypeptide of claim 1.

11. The nucleic acid of claim 10, operably linked to a promoter.

12. A vector comprising the nucleic acid of claim 10.

13. The vector of claim 12, wherein the vector is a viral vector.

14. A host cell transformed with the nucleic acid of claim 10.

15. A recombinant polypeptide comprising β1 and α1 domains of a human MHC class II molecule wherein the amino terminus of the α1 domain is covalently linked to the carboxy terminus of the β1 domain, and wherein the MHC class II molecule does not include an α2 domain or a β2 domain.

16. The recombinant polypeptide according to claim 15, wherein the polypeptide further comprises an antigenic determinant associated with the polypeptide by covalent or non-covalent interaction.

17. The recombinant polypeptide according to claim 16, wherein the antigenic determinant is covalently linked to the amino terminus of the β1 domain.

18. The recombinant polypeptide according to claim 15, wherein the polypeptide further comprises a detectable marker or toxic moiety.

19. A recombinant polypeptide comprising a human MHC class I α1 domain and a human MHC class I α2 domain, and wherein the amino terminus of the α2 domain is covalently linked to the carboxy terminus of the α1 domain, and wherein the MHC class I molecule does not include an α3 domain.

20. The recombinant polypeptide according to claim 19, wherein the polypeptide further comprises an antigenic determinant associated with the polypeptide by covalent or non-covalent interaction.

21. The recombinant polypeptide according to claim 20, wherein the antigenic determinant is covalently linked to the amino terminus of the α1 domain.

22. The recombinant polypeptide according to claim 20, wherein the polypeptide further comprises a detectable marker or toxic moiety.

23. A pharmaceutical composition comprising a polypeptide according to claim 1, and a pharmaceutically acceptable carrier.

24. A recombinant polypeptide comprising only two domains of a human MHC class II peptide, wherein the two domains are an al domain and a β1 domain, wherein the amino terminus of the al domain is covalently linked to the carboxy terminus of the β1 domain.

25. The polypeptide of claim 24, wherein the covalent linkage between the α1 and β1 domains is provided by a peptide linker sequence.

26. The purified MHC polypeptide of claim 24, wherein the MHC polypeptide is non-covalently associated with an antigen.

27. The purified MHC polypeptide of claim 24, wherein the MHC polypeptide is covalently associated with an antigen.

28. A recombinant polypeptide comprising only two domains of a human MHC class I peptide, wherein the two domains are an α1 domain and a α2 domain, wherein the amino terminus of the α2 domain is covalently linked to the carboxy terminus of the α1 domain.

29. The polypeptide of claim 28, wherein the covalent linkage between the β1 and α2 domains is provided by a peptide linker sequence.

30. The purified MHC polypeptide of claim 28, wherein the MHC polypeptide is non-covalently associated with an antigen.

31. The purified MHC polypeptide of claim 28, wherein the MHC polypeptide is covalently associated with an antigen.

32. A recombinant nucleic acid molecule, comprising first, second and third regions represented by the formula Pr-B-A, wherein:
Pr is a promoter sequence;
B is a coding sequence that encodes a β1 domain of a human MHC class II molecule; and
A is a coding sequence that encodes an al domain of a human MHC class II molecule;
wherein Pr is operably linked to B, and B and A comprise a single open reading frame.

33. A recombinant nucleic acid molecule, comprising first, second, third and fourth regions represented by the formula Pr-P-B-A, wherein:
Pr is a promoter sequence;
P is a coding sequence that encodes a peptide antigen;
B is a coding sequence that encodes a β1 domain of a human MHC class II molecule; and
A is a coding sequence that encodes an α1 domain of a human MHC class II molecule;
wherein Pr is operably linked to P, and P, B and A comprise a single open reading frame.

34. A recombinant nucleic acid molecule, comprising first, second and third regions represented by the formula Pr-B-A, wherein:
Pr is a promoter sequence;
B is a coding sequence that encodes an α1 domain of a mammalian MHC class I molecule; and
A is a coding sequence that encodes an α2 domain of a mammalian MHC class I molecule;
wherein Pr is operably linked to B, and B and A comprise a single open reading frame, and wherein the open reading frame does not encode an α3 domain of a mammalian MHC class I molecule.

35. A recombinant nucleic acid molecule, comprising first, second, third and fourth regions represented by the formula Pr-P-B-A, wherein:
Pr is a promoter sequence;
P is a coding sequence that encodes a peptide antigen;
B is a coding sequence that encodes an α1 domain of a human MHC class I molecule; and
A is a coding sequence that encodes an α2 domain of a human MHC class I molecule;
wherein Pr is operably linked to P, and P, B and A comprise a single open reading frame, and wherein the open reading frame does not encode an α3 domain of a mammalian MHC class I molecule.

36. A method for detecting or quantifying in a biological sample the presence of T-cells having a receptor specific for a specified antigen, comprising:
contacting the biological sample with a recombinant polypeptide comprising either (1) covalently linked β1 and α1 domains of a human MHC class II molecule wherein the carboxy terminus of the β1 domain is covalently linked to the amino terminus of the α1 domain, and further comprising the specified antigen bound in a peptide binding groove formed by the β1 and the α1 domain, or (2) a recombinant polypeptide comprising covalently linked α1 and α2 domains of a human MHC class I molecule wherein the carboxy terminus of the α1 domain is covalently linked to the amino terminus of the α2 domain, wherein the polypeptide does not include an α3 domain of a human MHC class I molecule and wherein the polypeptide further comprises the specified antigen bound in a peptide binding groove formed by the α1 and the α2 domain; and
detecting or quantifying the presence of specific binding of the recombinant polypeptide with said T-cells.

37. A method for reducing an immune response against an antigenic determinant in a subject, comprising:
administering a therapeutically effective amount of the polypeptide of claim 3, or of a nucleic acid encoding the polypeptide of claim 3; and
subsequently presenting the antigenic determinant to the subject, wherein administration of the polypeptide or the nucleic acid sequence reduces the immune response when the antigenic determinant is presented in the subject.

38. The method of claim 37, wherein the reduced immune response is a decrease in an influx or proliferation of a T cell, a macrophage, a B cell, or an NK cell.

39. The method of claim 37, wherein the reduced immune response is a reduction in the expression of a cytokine.

40. The method of claim 37, wherein the reduced immune response is an induction of a T suppressor cell response.

41. A method for inducing an immunoregulatory cell against an antigenic determinant, comprising administering a therapeutically effective amount of the polypeptide of claim 3 to the immunoregulatory cell; and
subsequently presenting the antigenic determinant to the immunoregulatory cell;
wherein the presentation of the antigenic determinant results in an induction of the immunoregulatory cell.

42. The method of claim 41, wherein the immunoregulatory cell reduces inflammation and cellular recruitment when the antigen is subsequently encountered with an immunogenic stimulus.

43. The method of claim 41, wherein the antigenic determinant is a tissue specific antigenic determinant.

44. The method of claim 41, wherein the immunoregulatory cell is induced as compared to a control.

45. The method of claim 41, wherein the immunoregulatory cell is in vivo.

46. The method of claim 41, wherein the immunoregulatory cell is in vitro.

47. A method for inducing the expression of a cytokine in a mammalian T cell, comprising contacting the T cell with an effective amount of the polypeptide of claim 3, thereby inducing the expression of the cytokine.

48. The method of claim 47, wherein the cytokine is IL-10.

49. The method of claim 47, wherein the cell is in vivo.

50. The method of claim 47, wherein the cell is in vitro.

51. A method of treating or preventing an immune-mediated disorder in a subject, comprising
administering to the subject a therapeutically effective amount of the polypeptide of claim 3 or of a nucleic acid encoding the polypeptide of claim 3;
wherein subsequent presentation of the antigenic determinant to an immune cell of the subject results in treatment or prevention of the immune-mediated disorder.

52. The method of claim 51, wherein the immune-mediated disorder is rheumatoid arthritis, chronic beryllium disease, insulin-dependent diabetes mellitus, throidititis, inflammatory bowel disease, uveitus, polyarteritis, Multiple Sclerosis or Myasthenia Gravis.

53. A pharmaceutical composition comprising the polypeptide of claim 3 in a pharmaceutically acceptable carrier.

54. A method of treating a disease caused by antigen-specific T-cells, comprising administering to a patient a composition comprising a polypeptide according to claim 3, or a nucleic acid encoding the polypeptide of claim 3, thereby treating the disease.

55. A method of activating a T cell in a subject, comprising administering a therapeutically effective amount of the polypeptide of claim 3, thereby activating the T cell.

56. The method of claim 55, wherein the subject is human.

57. The method of claim 55, wherein the T cell produces IL-10.

58. The method of claim 55, wherein the antigenic determinant is an antigenic determinant from a tumor cell.
